SEQUENCE LISTING (18 sequences)

SEQ ID NO: 1 - DNA, 27 bp, synthetic construct
  source 1..27: mol_type = other DNA; note = first shield sequence
gtgctcgcttcggcagcacatatacta

SEQ ID NO: 2 - DNA, 29 bp, synthetic construct
  source 1..29: mol_type = other DNA; note = second shield sequence
tctagagcggacttcggtccgcttttttt

SEQ ID NO: 3 - DNA, 22 bp, synthetic construct
  source 1..22: mol_type = other DNA; note = capture sequence
gctttaaggccggtcctagcaa

SEQ ID NO: 4 - DNA, 90 bp, synthetic construct
  source 1..90: mol_type = other DNA; note = sgRNA scaffold
gtttaagagctaagctggaaacagcatagcaagtttaaataaggctagtccgttatcaacttggccggccaagtggcaccgagtcggtgc

SEQ ID NO: 5 - DNA, 51 bp, synthetic construct
  source 1..51: mol_type = other DNA; note = F29 pRNA scaffold
ttgccatgtgtatcggtccgatactttgttgatggccatcaatcatggcaa

SEQ ID NO: 6 - DNA, 51 bp, synthetic construct
  source 1..51: mol_type = other DNA; note = F30 pRNA scaffold
ttgccatgtgtatcggtccgatactctgatgatggccatcattcatggcaa

SEQ ID NO: 7 - DNA, 182 bp, synthetic construct
  misc_difference 34..41: note = n is a, c, g, or t
  source 1..182: mol_type = other DNA; note = barcoded RNA with sgRNA scaffold
gtgctcgcttcggcagcacatatactagtcgacnnnnnnnngtttaagagctaagctggaaacagcatagcaagtttaaataaggctagtccgttatcaacttggccgctttaaggccggtcctagcaaggccaagtggcaccgagtcggtgctctagagcggacttcggtccgcttttttt

SEQ ID NO: 8 - DNA, 143 bp, synthetic construct
  misc_difference 34..41: note = n is a, c, g, or t
  source 1..143: mol_type = other DNA; note = barcoded RNA with f29 scaffold
gtgctcgcttcggcagcacatatactagtcgacnnnnnnnnttgccatgtgtatcggtccgatactttgttgatgggctttaaggccggtcctagcaaccatcaatcatggcaatctagagcggacttcggtccgcttttttt

SEQ ID NO: 9 - DNA, 143 bp, synthetic construct
  misc_difference 34..41: note = n is a, c, g, or t
  source 1..143: mol_type = other DNA; note = barcoded RNA with f30 scaffold
gtgctcgcttcggcagcacatatactagtcgacnnnnnnnnttgccatgtgtatcggtccgatactctgatgatgggctttaaggccggtcctagcaaccatcattcatggcaatctagagcggacttcggtccgcttttttt

SEQ ID NO: 10 - RNA, 27 nt, synthetic construct
  source 1..27: mol_type = other RNA; note = first shield sequence
gtgctcgcttcggcagcacatatacta

SEQ ID NO: 11 - RNA, 29 nt, synthetic construct
  source 1..29: mol_type = other RNA; note = second shield sequence
tctagagcggacttcggtccgcttttttt

SEQ ID NO: 12 - RNA, 22 nt, synthetic construct
  source 1..22: mol_type = other RNA; note = capture sequence
gctttaaggccggtcctagcaa

SEQ ID NO: 13 - RNA, 90 nt, synthetic construct
  source 1..90: mol_type = other RNA; note = sgRNA scaffold
gtttaagagctaagctggaaacagcatagcaagtttaaataaggctagtccgttatcaacttggccggccaagtggcaccgagtcggtgc

SEQ ID NO: 14 - RNA, 51 nt, synthetic construct
  source 1..51: mol_type = other RNA; note = F29 pRNA scaffold
ttgccatgtgtatcggtccgatactttgttgatggccatcaatcatggcaa

SEQ ID NO: 15 - RNA, 51 nt, synthetic construct
  source 1..51: mol_type = other RNA; note = F30 pRNA scaffold
ttgccatgtgtatcggtccgatactctgatgatggccatcattcatggcaa

SEQ ID NO: 16 - RNA, 182 nt, synthetic construct
  misc_difference 34..41: note = n is a, c, g, or t
  source 1..182: mol_type = other RNA; note = barcoded RNA with sgRNA scaffold
gtgctcgcttcggcagcacatatactagtcgacnnnnnnnngtttaagagctaagctggaaacagcatagcaagtttaaataaggctagtccgttatcaacttggccgctttaaggccggtcctagcaaggccaagtggcaccgagtcggtgctctagagcggacttcggtccgcttttttt

SEQ ID NO: 17 - RNA, 143 nt, synthetic construct
  misc_difference 34..41: note = n is a, c, g, or t
  source 1..143: mol_type = other RNA; note = barcoded RNA with F29 scaffold
gtgctcgcttcggcagcacatatactagtcgacnnnnnnnnttgccatgtgtatcggtccgatactttgttgatgggctttaaggccggtcctagcaaccatcaatcatggcaatctagagcggacttcggtccgcttttttt

SEQ ID NO: 18 - RNA, 143 nt, synthetic construct
  misc_difference 34..41: note = n is a, c, g, or t
  source 1..143: mol_type = other RNA; note = barcoded RNA with f30 scaffold
gtgctcgcttcggcagcacatatactagtcgacnnnnnnnnttgccatgtgtatcggtccgatactctgatgatgggctttaaggccggtcctagcaaccatcattcatggcaatctagagcggacttcggtccgcttttttt

(19) United States
(12) Patent Application Publication
(10) Pub. No.: US 2025/0354297 A1
(43) Pub. Date: Nov. 20, 2025

(54) SHIELDED SMALL NUCLEOTIDES FOR INTRACELLULAR BARCODING

(71) Applicant: BOARD OF REGENTS, THE UNIVERSITY OF TEXAS SYSTEM, Austin, TX (US)

(72) Inventors: Xiaoyin LU, Houston, TX (US); Shane LOFGREN, Houston, TX (US); Pawel MAZUR, Houston, TX (US)

(21) Appl. No.: 18/705,395

(22) PCT Filed: Oct. 31, 2022
(86) PCT No.: PCT/US2022/078994

§ 371 Date: Apr. 26, 2024

Related U.S. Application Data
(60) Provisional application No. 63/273,683, filed on Oct. 29, 2021.

(51) Int. Cl. C40B 40/06; C12N 5/0783; C12N 5/09; C12N 15/113; C12N 15/115; C12Q 1/6869; C12Q 1/6886; C40B 50/06

(52) U.S. Cl. CPC C40B 40/06; C12N 5/0636; C12N 5/0693; C12N 15/113; C12N 15/115; C12Q 1/6869; C12Q 1/6886; C40B 50/06; C12Q 2600/112

(57) ABSTRACT

The present disclosure relates to a barcoded RNA comprising a first shield sequence at the 5′ end of the barcoded RNA, a barcode sequence, a scaffold sequence, a capture sequence, and a second shield sequence at 3′ end of the barcoded RNA. The present disclosure also provides for methods of performing single-cell RNA sequencing using the barcoded RNA. The present disclosure also provides for libraries including the barcoded RNA.

### CROSS-REFERENCE TO RELATED APPLICATIONS

The present application claims the priority benefit of U.S. Provisional Patent Application No. 63/273,683, filed Oct. 29, 2021, which is incorporated herein by reference in its entirety.

### REFERENCE TO SEQUENCE LISTING SUBMITTED ELECTRONICALLY VIA EFS-WEB

The content of the electronically submitted sequence listing (Name: 4443_005PC01_Seqlisting_ST26.xml; Size: 23,740 bytes; and Date of Creation: Oct. 27, 2022) is herein incorporated by reference in its entirety.

### FIELD OF THE DISCLOSURE

The present disclosure relates to constructs and methods for single cell sequencing.

### BACKGROUND

Single-cell RNA sequencing provides transcriptional profiling of many, e.g., thousands, of individual cells. This level of throughput analysis can provide information at the single-cell level as to what genes are expressed, in what quantities, and how they differ across thousands of cells within a heterogeneous sample. Single-cell RNA sequencing has revolutionized the research of transcriptomics. However, high-throughput parallel profiling of multiple samples remains challenging due to limitations such as library preparation cost and batch effects. Multiplex single-cell RNA sequencing using oligo-tagged antibodies provides an option to label and pool different samples and load them simultaneously. Yet the approach is limited by the amount of ubiquitously cell surface markers and other technical challenges. For example, individual samples must be pooled immediately before loading. In addition, pre-pooling experiments for in vivo studies are not feasible using antibody-based labeling or other non-genetic barcoding methods.

Intracellular genetic barcoding has the potential to address some challenges. One promising strategy is polyA labeling using lentiviruses. However, this usually requires designing the entire expression cassette in reverse orientation and adding extra polyA signals. Moreover, a further step of dial-out polymerase chain reaction (PCR) needs to be introduced to recover the cell barcodes from the mRNA library, which demands customization and optimization.

Thus, although approaches have been developed to facilitate multiplex single-cell RNA sequencing, further exploration of alternative methods is motivated.

### BRIEF SUMMARY

Certain aspects of the present disclosure are directed to a barcoded RNA construct and methods of using the same to perform single cell RNA sequencing. In some aspects, the disclosure is directed to RNA molecules that comprise a shield sequence, a scaffold sequence, a barcode sequence, and a RNA capture domain, and methods of using the same.

In some aspects, provided herein is a barcoded RNA comprising a first shield sequence at the 5′ end of the barcoded RNA, a barcode sequence, a scaffold sequence, a capture sequence, and a second shield sequence at the 3′ end of the barcoded RNA.

In some aspects, the first shield sequence comprises at least one stem loop. In some aspects, the first shield sequence comprises a sequence at least 85%, at least 90%, at least 95%, at least 96%, at least 97%, at least 98%, at least 99%, or 100% identical to the nucleotide sequence set forth as SEQ ID NO: 10. In some aspects, the first shield sequence comprises the nucleotide sequence set forth as SEQ ID NO: 10.

In some aspects, the second shield sequence comprises at least one stem loop. In some aspects, the second shield sequence comprises a sequence at least 85%, at least 90%, at least 95%, at least 96%, at least 97%, at least 98%, at least 99%, or 100% identical to the nucleotide sequence set forth as SEQ ID NO: 11. In some aspects, the second shield sequence comprises the nucleotide sequence set forth as SEQ ID NO: 11.

In some aspects, the barcode sequence is 8 to 20 nucleotides long.

In some aspects, the scaffold sequence is a sgRNA. In some aspects, the scaffold sequence is a bacteriophage pRNA. In some aspects, the bacteriophage pRNA is F29. In some aspects, the bacteriophage pRNA is F30.

In some aspects, the capture sequence comprises a sequence at least 85%, at least 90%, at least 95%, at least 96%, at least 97%, at least 98%, at least 99%, or 100% identical to the nucleotide sequence set forth as SEQ ID NO: 12. In some aspects, the capture sequence comprises the nucleotide sequence set forth as SEQ ID NO: 12.

In some aspects, the barcoded RNA further comprises a terminator sequence.

In some aspects, the barcoded RNA further comprises a RNA aptamer. In some aspects, the RNA aptamer is a fluorescent RNA aptamer. In some aspects, the fluorescent RNA aptamer is a Broccoli RNA aptamer.

In some aspects, provided herein are methods of performing single-cell RNA sequencing. In some aspects, the methods of performing single-cell RNA sequencing comprise introducing a barcoded RNA library to a population of cells, performing single-cell RNA sequencing on the population of cells. In some aspects, the barcoded RNA construct comprises a first shield sequence at the 5′ end of the barcoded RNA, a unique barcode sequence, a scaffold sequence, a capture sequence, and a second shield sequence at the 3′ end of the barcoded RNA. In some aspects, the cells can be identified by the unique barcode sequence.

In some aspects, a subpopulation of cells can be identified when a plurality of cells comprise the same barcode sequence.

In some aspects, the barcoded RNA library is introduced to the population of cells by a viral vector. In some aspects, the viral vector is an adeno-associated viral (AAV) vector, an adenoviral vector, a lentiviral vector, or a retroviral vector.

In some aspects, provided herein is a polynucleotide comprising a promoter operably linked to a nucleic acid. In some aspects, the nucleic acid encodes a barcoded RNA sequence comprising a first shield sequence at the 5′ end of the barcoded RNA, a barcode sequence, a scaffold sequence, a capture sequence, and a second shield sequence at the 3′ end of the barcoded RNA.

In some aspects, the polynucleotide is a plasmid. In some aspects, the polynucleotide further comprises at least one restriction enzyme recognition sequence. In some aspects, the nucleic acid is positioned between two inverted terminal repeats (ITRs).

In some aspects, the promoter is a constitutively active promoter, a cell-type specific promoter, or an inducible promoter. In some aspects, the promoter is a Pol III promoter. In some aspects, the Pol III promoter is a U6 promoter.

In some aspects, the first shield sequence comprises at least one stem loop. In some aspects, the first shield sequence comprises a sequence at least 85%, at least 90%, at least 95%, at least 96%, at least 97%, at least 98%, at least 99%, or 100% identical to the nucleotide sequence set forth as SEQ ID NO: 10. In some aspects, the first shield sequence comprises the nucleotide sequence set forth as SEQ ID NO: 10.

In some aspects, the second shield sequence comprises at least one stem loop. In some aspects, the second shield sequence comprises a sequence at least 85%, at least 90%, at least 95%, at least 96%, at least 97%, at least 98%, at least 99%, or 100% identical to the nucleotide sequence set forth as SEQ ID NO: 11. In some aspects, the second shield sequence comprises the nucleotide sequence set forth as SEQ ID NO: 11.

In some aspects, the barcode sequence is 8 to 20 nucleotides long.

In some aspects, the scaffold sequence is a sgRNA. In some aspects, the scaffold sequence is a bacteriophage pRNA. In some aspects, the bacteriophage pRNA is F29. In some aspects, the bacteriophage pRNA is F30.

In some aspects, the capture sequence comprises a sequence at least 85%, at least 90%, at least 95%, at least 96%, at least 97%, at least 98%, at least 99%, or 100% identical to the nucleotide sequence set forth as SEQ ID NO: 12. In some aspects, the capture sequence comprises the nucleotide sequence set forth as SEQ ID NO: 12.

In some aspects, the polynucleotide further comprises a terminator sequence.

In some aspects, the polynucleotide further comprises a RNA aptamer. In some aspects, the RNA aptamer is a fluorescent RNA aptamer. In some aspects, the fluorescent RNA aptamer is a Broccoli RNA aptamer.

In some aspects, provide herein are methods of performing single-cell RNA sequencing comprising: (a) introducing a barcoded RNA library to a population of cells, wherein the barcoded RNA construct comprises (i) a first shield sequence at the 5′ end of the barcoded RNA, (ii) a unique barcode sequence, (iii) a scaffold sequence, (iv) a capture sequence, and (v) a second shield sequence at the 3′ end of the barcoded RNA; and (b) performing single-cell RNA sequencing on the population of cells, wherein the cells can be identified by the unique barcode sequence, and wherein an individual cell has a gene expression profile.

In some aspects, provided herein are methods of multiplexing samples for single cell sequencing comprising: (a) labeling single cells from a plurality of samples with a barcoded RNA, wherein the barcoded RNA comprises (i) a 5′ shield sequence, (ii) a barcode sequence, (iii) a scaffold sequence, (iv) a capture sequence, and (v) a 3′ shield sequence, wherein the barcode sequence comprises a unique barcode sequence and a cell of origin barcode sequence; (b) constructing a multiplexed single cell sequencing library for the plurality of samples comprising the cell of origin barcodes.

In some aspects, provided herein are methods of detecting a gene expression profile of a Chimeric Antigen Receptor T Cells (CAR-T Cells). In some aspects, the method of detecting a gene expression profile of a CAR-T cell comprises: (a) transducing a plurality of T cells with (i) a Chimeric Antigen Receptor (CAR) and (ii) at least one barcoded RNA construct to form a population of CAR-T cells, (b) subjecting the population of CAR-T cells to a test condition, (c) collecting the population of CAR-T cells after the test condition, (d) pooling the population of CAR-T cells and (e) performing single-cell RNA sequencing to determine a gene expression profile the barcoded CAR-T cells in the population. In some aspects, the barcoded RNA construct comprises a 5′ shield sequence, a unique barcode sequence, a scaffold sequence, a capture sequence, and a 3′ shield sequence. In some aspects, the unique barcode sequence allows for demultiplexing of the population of CAR-T cells.

In some aspects, the method further comprises selecting a CAR-T cell with a desired gene expression profile. In some aspects, the selected CAR-T cell is used to treat a patient. In some aspects, the selected CAR-T cell is used to treat a patient suffering from a bladder cancer. In some aspects, the selected CAR-T cell is used to mediate long-term CD4+ T cell responses in the presence of a chronic antigen.

In some aspects, the test condition is injection into a tumor in an animal model.

In some aspects, the gene expression profile displays genes involved in T cell activation. In some aspects, the gene expression profile displays genes involved in T cell exhaustion. In some aspects, the gene expression profile displays genes involved in apoptosis.

In some aspects, the T cells are transduced with a viral vector. In some aspects, the viral vector is an adeno-associated viral (AAV) vector, an adenoviral vector, a lentiviral vector, or a retroviral vector.

In some aspects, provided herein are methods of selecting a tumor infiltrating immune cell from a patient. In some aspects, the method of selecting a tumor infiltrating immune cell from a patient comprises isolating tumor infiltrating immune cells from a patient, introducing a barcoded RNA construct to a tumor infiltrating immune cell, challenging the tumor infiltrating immune cells with cancer cells, collecting the tumor infiltrating immune cells after the challenge, pooling the population of tumor infiltrating immune cells, performing single-cell RNA sequencing to determine a gene expression profile for the tumor infiltrating immune cell, and selecting a tumor infiltrating immune cell with the gene expression profile desired for treatment of the patient.

In some aspects, the barcoded RNA construct comprises a 5′ shield sequence, a unique barcode sequence, a scaffold sequence, a capture sequence, and a 3′ shield sequence. In some aspects, the unique barcode sequence allows for demultiplexing of the population of CAR-T cells.

In some aspects, the barcoded RNA construct is introduced to the tumor infiltrating immune cell by a viral vector. In some aspects, the viral vector is an adeno-associated viral (AAV) vector, an adenoviral vector, a lentiviral vector, or a retroviral vector. In some aspects, the method further comprises administrating the selected tumor infiltrating immune cells to a patient.

In some aspects, provided herein are methods for analyzing tumor development. In some aspects, the method for analyzing tumor development comprises introducing at least one barcoded RNA construct to a population of cancer cells to form a sample population, injecting the sample population into an animal model, allowing a tumor to develop in the animal model, isolating the tumor from the animal model, performing single-cell RNA sequencing on cells in the tumor. In some aspects, the barcoded RNA construct comprises a 5′ shield sequence, a unique barcode sequence, a scaffold sequence, a capture sequence, and a 3′ shield sequence. In some aspects, the unique barcode sequence allows for demultiplexing of the sample.

In some aspects, the at least one barcoded RNA construct is introduced to the population of cancer cells by a viral vector. In some aspects, the viral vector is an adeno-associated viral (AAV) vector, an adenoviral vector, a lentiviral vector, or a retroviral vector.

In some aspects, provided herein are methods for analyzing oncogenes. In some aspects, the method for analyzing oncogenes comprises introducing a viral vector to an animal model, allowing a tumor to develop in the animal model, isolating the tumor from the animal model, performing single-cell RNA sequencing on cells in the tumor. In some aspects, the viral vector comprises a unique oncogene and a barcoded RNA construct. In some aspects, the barcoded RNA construct comprises a 5′ shield sequence, a unique barcode sequence, a scaffold sequence, a capture sequence, and a 3′ shield sequence. In some aspects, the unique barcode sequence allows for demultiplexing of the sample.

In some aspects, the viral vector is an adeno-associated viral (AAV) vector, an adenoviral vector, a lentiviral vector, or a retroviral vector.

In some aspects, provided herein are cells expressing a barcoded RNA. In some aspects, the cell expressing a barcoded RNA comprises a first shield sequence at the 5′ end of the barcoded RNA, a barcode sequence, a scaffold sequence, a capture sequence, and a second shield sequence at the 3′ end of the barcoded RNA.

In some aspects, provided herein is a library comprising a plurality of barcoded RNAs. In some aspects, the library comprises a plurality of barcoded RNAs in which the barcoded RNA comprises a first shield sequence at the 5′ end of the barcoded RNA, a barcode sequence, a scaffold sequence, a capture sequence, and a second shield sequence at the 3′ end of the barcoded RNA.

In some aspects, the library comprises at least 100 unique barcoded RNAs. In some aspects, the library comprises at least 1000 unique barcoded RNAs. In some aspects, the library comprises at least 10000 unique barcoded RNAs.

In some aspects, the library is a viral library. In some aspects, the viral library is a lentiviral library.

In some aspects, provided herein are methods of multiplexing samples for single cell sequencing. In some aspects, the method of multiplexing samples for single cell sequencing comprises labeling single cells from a plurality of samples with a barcoded RNA, and constructing a multiplexed single cell sequencing library for the plurality of samples comprising the cell of origin barcodes. In some aspects, the barcoded RNA comprises a 5′ shield sequence, a barcode sequence, a scaffold sequence, a capture sequence, and a 3′ shield sequence. In some aspects, the barcode sequence comprises a unique barcode sequence and a cell of origin barcode sequence.

In some aspects, the method further comprises sequencing the library and demultiplexing in silico based on the cell of origin barcodes and the unique barcode sequence.

In some aspects, provided herein are kits comprising expression constructs. In some aspects, the expression construct comprises a promoter operably linked to a nucleic acid encoding a barcoded RNA sequence. In some aspects, the barcoded RNA sequences comprises a first shield sequence at the 5′ end of the barcoded RNA, a barcode sequence, a scaffold sequence, a capture sequence, and a second shield sequence at the 3′ end of the barcoded RNA.

In some aspects, the expression construct is a plasmid.

In some aspects, the expression construct further comprises at least one restriction enzyme recognition sequence.

In some aspects, the kit comprises a library. In some aspects, the library comprises a plurality of barcoded RNAs. In some aspects, the barcoded RNA comprises a first shield sequence at the 5′ end of the barcoded RNA, a barcode sequence, a scaffold sequence, a capture sequence, and a second shield sequence at the 3′ end of the barcoded RNA.

In some aspects, the barcoded RNA is stable in the absence of Cas9.

In some aspects, the barcoded RNA comprises a first arm and a second arm, wherein the second arm comprises the barcode sequence.

In some aspects, the method further comprises amplifying the barcoded RNA after introduction to the population of cells, wherein the amplification comprises a primer specific to the first shield sequence.

In some aspects, the population of cells are primary T cells.

In some aspects, provided herein is a method of transcriptional profiling, the method comprising a) introducing a barcoded RNA library to a population of cells, wherein the barcoded RNA construct comprises (i) a first shield sequence at the 5′ end of the barcoded RNA, (ii) a unique barcode sequence, (iii) a scaffold sequence, (iv) a capture sequence, and (v) a second shield sequence at the 3′ end of the barcoded RNA; b) performing single-cell RNA sequencing on the population of cells; and c) lineage-tracing and transcriptional profiling the individual cell of the population of cells. In some aspects, the cell can be identified by the unique barcode sequence. In some aspects, an individual cell has a gene expression profile.

In some aspects, the individual cell comprises a unique genotype compared to a genotype of the population of cells.

In some aspects, over 90% of the individual cells in the population of cells are transcriptionally profiled.

In some aspects, the barcoded RNA or the barcoded RNA constructs used in the methods disclosed herein comprise any of the barcoded RNAs disclosed herein.

### DESCRIPTION OF FIGURES

FIGS. 1A-1B shows exemplary shielded nucleotides for multiplexed single-cell RNA sequencing. FIG. 1A shows a shielded sgRNA. FIG. 1B shows a shielded F30-derived small RNA.

FIG. 2 shows conventional sgRNA expression levels in the presence of cells having low and high levels of dCas9 (endonuclease deficient Cas9) or in the absence of dCas9 (sgRNA). RNAs were extracted from K562 cells and were subjected to qPCR. The data is presented as means±SD (n=3).

FIG. 3 shows a comparison of shielded sgRNA constructs and conventional sgRNAs normalized expression levels. The constructs included shielded sgRNA with an 8 nucleotide protospacer, shielded sgRNA with a 20 nucleotide protospacer, conventional sgRNAs analyzed in the presence of Cas9 (sgRNA+dCas9), and conventional sgRNAs in the absence of Cas9 (sgRNA). RNAs were extracted from indicated K562 cells and were subjected to qPCR. The data are presented as means±SD (n=3).

FIG. 4 shows the number of sample barcodes captured per cell using different sgRNA constructs. sgRNA without Cas9 (sgRNA), sgRNA with Cas9 (sgRNA_Cas9), shielded sgRNA with a 20-nucleotide protospacer, and shielded sgRNA with an 8-nucleotide protospacer were analyzed. The data represents median barcode counts per cell for cells bearing conventional sgRNAs or the shielded sgRNAs.

FIG. 5 shows the number of sample barcodes captured per cell using different constructs. Shielded sgRNA and four shielded F30-derived small RNA constructs (F30-CapArm1, F30-Broccoli-CapArm2, F30-CapArm1-Broccoli, and F30-CapArm2) were analyzed. The data represents median barcode counts per cell for cells bearing the shielded sgRNAs or the shielded F30-derived small RNAs.

FIG. 6 shows the number of sample barcodes captured per cell using different constructs across different cell types. F30-derived small RNA constructs, shielded sgRNA constructs, and a convention sgRNA in the presence of Cas9 were analyzed. The different cell types included human primary T cells (hT), K562 cells, Aspc-1 cells and mouse lymphoma EL4 cells (EL4). The data represents median barcode counts per cell.

FIG. 7 displays relative fluorescence against the number of events for T-cell exhaustion-related cell surface proteins in CD19-28z and GD2-28z CAR-T cells. FLOW cytometry analysis of immune checkpoint inhibitors on CD19-28z and GD2-28z CAR-T cells bearing shielded sgRNAs were performed. The immune checkpoint inhibitors analyzed included PD-1, LAG-3, TIM-3, and CD39.

FIG. 8 shows the change in exhaustion-related genes between CD19-28z and GD2-28z CAR-T cells as measured by single-cell RNA sequencing. The Log₂ Fold Change was mapped against the −Log₁₀ False Discovery Rate.

FIG. 9 shows 10 differentially expressed genes between CD19-28z and GD2-28z CAR-T cells as measured by single-cell RNA sequencing. The Log₂ Fold Change was mapped against the −Log₁₀ False Discovery Rate.

FIG. 10 shows an experimental method for in vivo immune cell adoptive transfer experiments using Shielded Small Nucleotide-seq (SSN-seq). SSN-labelled CAR-T cells with different genetic edits are transferred to mice with tumors. The tumor infiltrating CAR-T cells are isolated, and sc-RNA sequencing analysis is performed for edit-phenotype mapping. This figure was created with BioRender.com.

FIG. 11 shows an experimental method for personalized medicine using SSN-seq. Tumor infiltrating immune cells with unique B cell receptor (BCR) and T cell receptor (TCR) signatures are isolated from a patient. SSN-labelled immune cells carrying a personalized BCR/TCR library are transferred to mice with tumors. sc-RNA sequencing analysis is performed for therapeutic prioritization. This figure was created with BioRender.com.

FIG. 12 shows an experimental method for an in vivo transplant-based tumor model using SSN-seq. Cancer cells with different genetic variations are multiplexed with SSNs. The labelled cancer cells are injected into a mouse model, upon which a tumor is allowed to develop. The tumor is isolated and analyzed by sc-RNA sequencing. This figure was created with BioRender.com.

FIG. 13 shows an experimental method for an in vivo autochthonous tumor model using SSN-seq. Viral vectors loaded with different oncogenes and barcodes are injected into mice. Tumors are allowed to develop. The tumors are isolated and sc-RNA sequencing analysis is performed. This figure was created with BioRender.com.

FIG. 14A shows a schematic of a sgRNA with integrated capture sequence (CS1) to enable direct-capture by gel beads using Chromium Single Cell 3′ Reagent Kits v3 with Feature Barcoding technology workflow. GBC, group barcode.

FIG. 14B shows a schematic of sgRNA and mRNA sequencing libraries. The two types of libraries are generated simultaneously from the same single cell using primers specific to the capture sequence (CS1) primer and the poly (dT) following 10× Genomics single cell 3′ gene expression with feature barcoding technology workflow. CBC, cell barcode. UMI, unique molecular identifier. GBC, group barcode. TSO, template switch oligo. The TSO adds a common 5′ sequence to full-length cDNA after reverse transcription.

FIG. 14C shows a uniform manifold approximation and projection (UMAP) analysis of single-cell expression profiles of the mixed human and mouse cells expressing distinct sgRNA (n=10,245). Transcriptome-based clustering reveals distinct cell populations (left panel). Corresponding sgRNA-based assignment projected on the UMAP plot (right panel). Human primary T cells were isolated from peripheral blood mononuclear cells (PBMCs). Mouse primary T cells were isolated from C57BL/6 mouse spleens. AsPC-1, a human pancreatic cancer cell line. KPC, a cell line derived from KrasLSLG12D/+; Trp53LSL-R172H/+; PtflaCre/+ pancreatic cancer mouse model. KPC_PDL1, KPC cells modified to overexpress CD274 encoding mouse PDL1.

FIG. 14D shows a percentage of cells recovered for each cell type, normalized by the transduction efficiency for each cell input (determined by NGFR+%).

FIG. 14E shows a quantification of sgRNA UMI counts per guide-assigned cell in indicated experimental group. The center line indicates the median and the box marks the 25th and 75th percentiles. The whiskers extend to the smallest/largest value no further than 1.5 times the interquartile range. Outliers beyond the whisker ends are not plotted. The number above each plot indicates the median number of sgRNA UMIs within the corresponding group.

FIG. 14F shows an immunoblot analysis with indicated antibodies of cell lysates isolated from AsPC-1 or K562 cells transduced to express doxycycline-inducible Flag-tagged dCas9 and sgRNA with group-specific barcodes, in the absence or presence of doxycycline (Dox). Tubulin is shown as a loading control.

FIG. 14G shows a qPCR detection of sgRNA expression levels in control and dCas9-expressing AsPC-1 or K562 cells as in (FIG. 14F). CS1-specific primers are used to reverse transcribe the sgRNAs. qPCR primers are designed to amply the constant region of the sgRNAs. Data are mean±s.d. of three biologically independent experiments/biological replicates. P values determined by two-tailed unpaired t-test.

FIG. 14H shows a schematic of a shielded small nucleotide sgRNA (SSN.guide) with two shield sequences at the 5′ and 3′ ends.

FIG. 14I shows a qPCR detection of sgRNA and SSN-guide abundance in AsPC-1, K562 and human primary T cells. Data are mean±s.d. of three biologically independent experiments/biological replicates. P values determined by two-tailed unpaired t-test.

FIG. 14J shows a schematic of a shielded small nucleotide based on F30 scaffold (SSN.F30) with the capture sequence inserted into the Arm1 or Arm2 stem loops.

FIG. 14K shows an experimental approach to test SSN.guide and SSN.F30 performance using in K562 cells.

FIG. 14L shows a quantification of group barcode (GBC) UMI counts in indicated experimental groups as in (FIG. 14K). The number above each violin plot indicates the median number of GBC UMI counts. The group barcode length is either 20 nt (for STD.guide and SSN.guide.20nt) or 8 nt (for SSN.guide.8nt, SSN.F30.Arm1 and SSN.F30.Arm2).

FIG. 15A shows a schematic overview of the multiplex scRNA-seq with SSN barcoding in the twenty experimental groups using K562, AsPC-1, EL4 and human T cells: G01 and G02 express standard sgRNAs±Cas9, G03-G07 contain U6-SSN.guide inserted in sense orientation; in G08-G09 U6-SSN.guide is inserted in reverse orientation (denoted as “U6rev”), G10-G12 express modifications of SSN.F30 in K562 cells; G13-G20 groups express U6-SSN in sense orientation: U6-SSN.guide or U6-SSN.F30 with CS1 inserted to Arm 2 in AsPC-1 (G13-G14) EL4 (G15-G16) and in human primary T cells (G17-G20). Grey font denotes groups with suboptimal performance.

FIG. 15B shows a flow cytometry expression analysis of indicated immune checkpoints in CD19.28z (G19) and HA.GD2.28z (G20) CAR T cell groups.

FIG. 15C shows a quantification of group barcode UMI counts in indicated experimental groups as in (FIG. 15A). The number above each violin plot indicates the median number of UMI counts.

FIG. 15D shows a UMAP analysis of multiplexed single-cell RNA expression profiles (n=10,359) indicates that cells group according to lineage identity (left panel). UMAP visualization of cells based on barcode assignment demonstrates cell identification accuracy using SSN barcoding (right panel). Multiplets were removed for visualization purposes.

FIG. 15E shows the percentage of each SSN or control barcode assigned cells in indicated experimental groups as in (FIG. 15A). The barcode counts distribution is proportional to input cell number demonstrating homotypic identification accuracy by SSN.

FIG. 15F shows a UMAP analysis of human CD8+ T cells (n=461) from (FIG. 15D) indicates that cells group into three sub-clusters.

FIG. 15G shows a distribution of CD8+ cells sub-clusters in each experimental group.

FIG. 15H shows a heat map of differentially expressed genes in each CD8+ T cell sub-cluster. The top 50 statistically significant (false discovery rate (FDR)<0.05 determined by Wilcoxon rank-sum test) differentially up-regulated genes for cluster C3 are shown. Representative genes related to T cell exhaustion are labeled on the right. Each cluster was randomly downsampled to include equal number of cells (74 cells per cluster) for visualization purposes.

FIG. 16A shows schematics of the standard and optimized (enrichSSN) approach for sequencing library preparation. The protocol follows the “Feature PCR” step described in the 10× workflow of 3′ gene expression with feature barcoding technology to PCR amplify sgRNA using the template-switch-oligo (TSO). In enrichSSN amplification 5′ shield sequence-specific primer is utilized generating a specific amplicon.

FIG. 16B shows a schematic of the dual SSN barcoding lentiviral vector for generation of CD19.28z CAR T cells. hU6, human U6 promoter. mU6, mouse U6 promoter.

FIG. 16C shows an experimental overview of the 8-plex CAR T SSN-seq experiment. SSN-labeled CAR T cells were cultured in the presence of DMSO (Mock), TWS119 (GSK3b inhibitor), JQ1 (BET inhibitor) or the combination of these two inhibitors (Combo). In addition, CAR T cell growth media was supplemented with IL2 or IL7 plus IL15 (IL7×15) for each drug treatment group.

FIG. 16D shows a flow cytometry analysis of T cell memory markers CCR7 and CD45RA in all experimental groups of CAR T cells.

FIG. 16E shows the percentage of SSN barcode-assigned cells per treatment group in the pooled CAR T infusion product.

FIG. 16F shows a heat map of differentially expressed genes in each treatment group. The top 10 statistically significant (FDR<0.05 determined by Wilcoxon rank-sum test) differentially up-regulated genes for each treatment group are shown. Representative T cell memory/effector genes are labeled on the right.

FIG. 16G shows a transcriptome-based UMAP clustering of single-cell expression profiles (n=9,854). Cells are colored based on cell cluster classifications (left panel). The four CD8+ clusters (C01-C04) and the four CD4+ clusters (C07-C10) comprise around 91% of all cells. Corresponding SSN barcode assignment of each treatment groups projected on the UMAP plot (right panel).

FIG. 16H shows a single-cell identification of subpopulations with bulk sample phenotype correlation (Scissor) analysis. A pseudo-bulk expression matrix and corresponding clinical phenotypes were derived from scRNA-seq of CD19 CAR T cell infusion products (axi-cel) coupled with clinical response of patients with large B cell lymphomas. The Scissor predicted response (CR: complete response; PR/PD: partial response or progressive disease) is overlayed on UMAP visualization as in (FIG. 16G). The cells not associated with the phenotype of interest are denoted as background cells.

FIG. 16I shows an expression analysis of genes associated with the Scissor predicted clinical response. Percentage of cells expressing indicated genes and relative levels of expression are shown.

FIG. 16J shows the proportion of CAR T cells associated with the Scissor-predicted clinical response (CR: complete response; PR/PD: partial response or progressive disease) and the ratio of CR to PR/PD cells in indicated treatment modality.

FIG. 16K shows an enrichment analysis (Fisher's exact test) of each treatment group within the indicated Scissor-predicted populations. The two mock groups displayed strong enrichment in PR/PD populations, while the six groups treated with different small molecule inhibitors showed enrichment in CR populations. Color of the dots indicates the log2-transformed odds ratio (blue: <0, underrepresented; red: >0, overrepresented). Dot size indicates the −Log₁₀-transformed false discover rate (FDR) calculated using Benjamini-Hochberg correction.

FIG. 17A shows a schematic overview of a proof-of-concept in vivo CAR T cells profiling experiment using SSN-seq. The pooled CAR T cells infusion product (8-plex CAR T, see FIG. 16C) was injected into NALM6-bearing NSG mice (Day 0). The animals were rechallenged with NALM6 tumor cells 21 days after the CAR T cells administration. CAR T cells were isolated by FACS (CD45+NGFR+ cells) from the spleens of treated mice 21 days after tumor rechallenge (Day 42). Sorted cells from three mice were pooled and processed for 3′ single-cell RNA-seq with feature barcoding and enrichSSN barcode amplification.

FIG. 17B shows the UMI counts for each SSN-assigned CAR T cell in the infusion product or in the in vivo CAR T cells, grouped by treatment. The center line indicates the median and the box marks the 25th and 75th percentiles. The whiskers extend to the smallest/largest value no further than 1.5 times the interquartile range. Outliers beyond the whisker ends are not plotted. The number above each violin plot indicates the median number of SSN barcode UMI counts.

FIG. 17C shows a SSN-seq successfully retrieved all eight treatment groups. The percentage of SSN barcode-assigned cells per treatment group in the in vivo pooled CAR T experiment is shown.

FIG. 17D shows the composition changes in total, CD8+ and CD4+ CAR T cells in the infusion product (Day 0) versus persistent CAR T cells retrieved after in vivo tumor rechallenge (Day 42).

FIG. 17E shows a transcriptome-based UMAP analysis of single-cell expression profiles of in vivo CAR T cells (n=8,679) identified 12 distinct clusters (C01-C12). Overlayed on UMAP cluster project are: expression of CD8A, CD4, dysfunction score (adapted from a previous study) and the Scissor predicted anti-PD-1 response (based on T cell dataset from melanoma patients with classified immunotherapy response).

FIG. 17F shows CAR T cell clusters (C01-C12) with a corresponding heat map of the top 20 statistically significant (FDR<0.05 determined by Wilcoxon rank-sum test) differentially expressed genes with list of key makers with biological implications (left panel), the percentage of cells which transcriptional profile associates with the Scissor predicted positive and negative clinical response to anti-PD-1 therapy (middle panel), and distribution of CAR T cells from each treatment group (right panel).

FIG. 18A shows a schematic overview of the multiplexed scRNA-seq experiment using standard direct-capture compatible sgRNA (STD.guide) as group barcode carrier. Cells in each experimental group were lentivirally transduced to express unique sgRNAs (denoted as sgRNA-1 to -5) and a cell surface marker NGFR.

FIG. 18B shows a quantification of indicated parameters in the multiplexed scRNA-seq experiments (as in FIG. 18A). sgRNA assignment rates for each cell type is shown. Lentivirus transduction efficiency for each group was determined by a cell surface marker NGFR+ expression using flow cytometry.

FIG. 18C shows a cell doublets prediction analysis (by DoubletFinder) of sgRNA-1 (human primary T cells) group indicates that majority of misassigned population is formed by transcriptionally distinct (heterotypic) cell doublets.

FIG. 18D shows violin plots of mouse PDL1 expression levels in control KPC cells (KPC_WT) and PDL1-overexpressing KPC cells (KPC_PDL1).

FIG. 18E shows differentially expressed genes in control KPC (KPC_WT) and PDL1-overexpressing KPC cells (KPC_PDL1). Adjusted P values were calculated by Wilcoxon rank-sum test with Bonferroni's correction. Red dots indicate significant genes with adjusted P-values<0.05 and Log2 (fold change)>0.25.

FIG. 19A shows schematics of all sgRNA- and F30-based SSN barcode designs tested in K562 cells. The control groups G01_STD.guide_mock and G02_STD.guide_Cas9 were transduced with lentiviral vector expressing standard (without SSN) sgRNA (STD.guide)+dCas9, respectively. G03_SSN.guide_A group expresses sgRNA-based SSN (SSN.guide) with 20 nt group barcode (GBC) whereas G04-G07 groups have 8 nt GBC. Cells in groups G08-G09 express SSN.guide cassette in the reverse orientation. G10_SSN.F30_A, cells express F30-based SSN barcode (SSN.F30) where the capture sequence is inserted into the Arm1 and in G11-G12 groups the capture sequence is inserted into the Arm2. G04-G07, G08-G09 or G11-G12 express GBC with different nucleotide sequence to assess an effect of barcode sequence on performance within the same barcode groups.

FIG. 19B shows the Pearson correlation analysis of transcription profiles between the indicated SSN-barcoded groups indicates that within the same cell type there are no signs of transcriptome perturbation due to expression of different SSN barcodes. Of note, human HA.GD2.28z CAR T cells group (G20) exhibits lower correlation with CD19.28z CAR T cells (G19) or primary human T cell groups (G18-G17) consistent with the observed enhanced exhaustion phenotype (see FIG. 15B). Group combining all identified cell multiples exhibits high transcriptome correlation with K562 groups suggesting that K562 cells are main contributors. The “not assigned” group is highly correlated with human primary T cells congruent with the observed overall lower SSN-assignment rate (see FIG. 15E).

FIGS. 19C-19D show quantification of RNA content in each cell type as determined by the number of total RNA UMI counts per cell. Analysis for mouse KPC, human AsPC-1, mouse primary T and human primary T cells (as in FIG. 14C) (FIG. 19C) and human K562, human AsPC-1, mouse EL4 and human primary T cells (as in FIG. 15D) (FIG. 19D). The number above each violin plot indicates the median number of total RNA UMI counts. Note that primary T cells have significantly lower total RNA UMI counts per cell. Part of FIGS. 14-19 were created with BioRender.com.

FIG. 20A shows a comparison of two strategies for SSN library preparation (the same cDNA input of SSN.guide-labeled human primary T cells). Schematics of the standard and optimized (enrichSSN) approach for library preparation with 10× Genomics Feature PCR or optimized enrichSSN amplification and the final library construction. The final SSN-seq libraries quality control was performed using Agilent Bioanalyzer. DNA electropherograms of the final libraries with indicated (arrows) expected product sizes: ˜310 bp with 10× Feature PCR or ˜280 bp with enrichSSN amplification (due to the omission of the TSO sequence). CBC, cell barcode. UMI, unique molecular identifier. CS1, 10× Capture Sequence 1. GBC, group barcode. TSO, template switch oligo. The TSO adds a common 5′ sequence to full-length cDNA after reverse transcription.

FIG. 20B shows quantification of SSN UMI counts per cell using standard Feature PCR or enrichSSN amplification for the final library preparation. Total sequencing reads and sequencing saturation were shown. Higher sequencing saturation indicates that a larger fraction of the library complexity has been captured. To enable fair comparison, only cells bearing the same cell barcodes were included. The enrichSSN strategies generated higher SSN UMI counts per cell. The center line indicates the median and the box marks the 25th and 75th percentiles. The whiskers extend to the smallest/largest value no further than 1.5 times the interquartile range. Outliers beyond the whisker ends are not plotted. The number to the right of each violin plot indicates the median number of UMIs counts.

FIG. 21 shows representative flow cytometry plots showing CAR T cells gated on the lymphocytes (SSC-A/FSC-A), singlets (FSC-H/FSC-A, SSC-H/SSC-A), live (7-AAD-), and a cell surface reporter NGFR+ populations.

FIG. 22A shows DNA electropherograms (Bioanalyzer traces) of the final dual SSN-barcode libraries of the 8-plex CAR T cells analyzed at the time of infusion and isolated from in vivo tumor model. Expected product sizes peaks indicated (arrows) at ˜250 bp (SSN.F30) and ˜280 bp (SSN.guide). The size difference is due to a shorter length between the GBC and the capture sequence in the F30 scaffold compared to the one in the sgRNA scaffold.

FIG. 22B shows total sequencing reads and sequencing saturation for SSN libraries from CAR T cells at the time of infusion and isolated from in vivo tumor model, indicating comparable sequencing depth and similar library complexity.

FIG. 23A shows the number of CAR T cells identified by SSN.guide and/or SSN.F30 barcoding (88% and 84%, respectively) indicating similar performance of both barcoding strategies.

FIG. 23B shows the expression analysis of selected markers of T cell effector (GZMB, GZMK, NKG7) and memory (CCR7, SELL, TCF7) functions for each CAR T cell treatment group (as in FIG. 16F).

FIG. 23C shows the distribution of CAR T cells from each treatment group in the identified gene clusters as in FIG. 16G based on SSN-seq analyses of pooled CAR T infusion product. The dot plots on the right indicate the corresponding expression of CD8A or CD4 in each cluster. The four CD8+ clusters (C01-C04) and the four CD4+ clusters (C07-C10) include approximately 91% of analyzed cells. Of note, clusters C01+C07 contain mainly TWS119-treated, C03+C09 JQ1-treated, C02+C08 are dominated by Combo-treated and C04+C10 are mainly contributed by Mock control groups.

FIG. 23D shows the identification of active gene signatures in in vivo CAR T cells clusters using AUCell algorithm. AUCell analysis of curated gene sets from the MSigDB Immunologic Signatures database: genes down-regulated in naïve vs. effector CD8 T cells (GSE10239) and naïve vs. activated CD4 T cells (GSE28726) identified significant downregulation of naïve and elevation of activation/effector T cell signatures in clusters C04 and C10 containing mostly the mock-treated cells.

FIG. 24 shows volcano plots of differentially expressed genes (DEGs) in CAR T cells undergoing the same drug-treatment regimen (TWS119, JQ1 and TWS119+JQ1 (Combo), or DMSO (Mock) controls) but were expanded in culture media supplemented with IL2 or IL7 plus IL15 (IL7×15) cytokines as indicated. Adjusted P-values were calculated by Wilcoxon rank-sum test with Bonferroni's correction. Red dots indicate significant genes with adjusted P-values<0.05 and Log2 (fold change)>0.25. The number of significantly altered genes is shown in red. Note that a limited number of DEGs can be identified, in accordance with cluster classification shown in FIG. 16G and FIG. 23C.

FIG. 25 shows CAR T cells isolated from single-cell suspensions of dissociated spleens of NALM6 leukemia mice at day 42 using FACS with flow plots gated on the lymphocytes (SSC-A/FSC-A), singlets (FSC-H/FSC-A), live (7-AAD-), CD45+ and NGFR+ populations.

FIG. 26A shows a quantification of the in vivo expansion advantage of each CAR T cell treatment group normalized to Mock_IL2. The fold change is corrected by their input cell proportion (calculated by the distribution in the infusion product).

FIGS. 26B-26C show an enrichment analysis (Fisher's exact test) of each treatment group within the indicated CD4+ or CD8+ populations of CAR T cells in vivo (FIG. 26B) or the infusion product (FIG. 26C). The odds ratio indicates the ratio of two sets of odds: odds of a treatment group presenting in one cluster shown in FIG. 17E versus the odds of the treatment group presenting in the remaining cells of all other clusters. Color of the dots indicates the Log2-transformed odds ratio (blue: <0, underrepresented; red: >0, overrepresented). Dot size indicates the −Log10-transformed false discover rate (FDR) calculated using Benjamini-Hochberg correction.

FIG. 26D shows the CD8+/CD4+ composition changes in the CAR T infusion product (Day 0) versus persistent CAR T cells retrieved after in vivo tumor rechallenge (Day 42).

FIG. 27A shows expression analysis of selected markers of T cell lineage (CD4, CD8A), memory (TCF7, CCR7) and effector (GZMB, GZMK, NKG7, PRF1) functions, chemokines (CXCL13), cytokines (IFNG), immune checkpoints (PDCD1, LAG3, HAVCR2, ENTPD1, TIGIT, CTLA4) and transcription factors (TOX, TOX2, EOMES, TBX21) for each cell cluster from the in vivo 8-plex CAR T cells experiment.

FIG. 27B shows enrichment analysis (Fisher's exact test) of in vivo CAR T cells from each treatment group in the identified 12 phenotypic clusters. Color of the dots indicates the Log2-transformed odds ratio (blue: <0, underrepresented; red: >0, overrepresented). Dot size indicates the −log10-transformed false discover rate (FDR) calculated using Benjamini-Hochberg correction.

### DETAILED DESCRIPTION

The present disclosure provides a barcoded RNA for single-cell RNA sequencing. In some aspects, the barcoded RNA can enable multiplexing within single-cell RNA sequencing. In some aspects, a polynucleotide of the disclosure comprises the barcoded RNA. Some aspects of the disclosure are directed to a library of barcoded RNAs. Some aspects of the disclosure are directed to a kit comprising a barcoded RNA or a library of barcoded RNAs.

In certain aspects of the disclosure, small RNAs such as sgRNAs can be engineered as sample barcodes for multiplex labeling in single-cell RNA sequencing. Using Shielded Small Nucleotide-seq (SSN-seq) for intracellular barcoding cells can allow for multiplexed single-cell RNA sequencing. In some aspects of the disclosure, modules including either a sgRNA scaffold or a bacteriophage pRNA scaffold can be used to link a sample barcode to a capture sequence, together with an anti-degradation motif. In some aspects, a SSN-seq can be characterized using multiple cell types including human primary T cells. In some aspects, the SSN-seq can achieve efficient sample assignments, promoting cell profiling in a cost-effective “label-pool-demultiplex” way.

While the present invention is described herein with reference to illustrative aspects for particular applications, it should be understood that the invention is not limited thereto. Those skilled in the art with access to the teachings herein will recognize additional modifications, applications, and aspects within the scope thereof and additional fields in which the invention would be of utility.

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

As used herein, the term “barcode sequence” refers to a short sequence of nucleotides (for example, DNA or RNA) that can be used as an identifier. In some aspects, the barcode sequence is an identifier for a known corresponding sequence, e.g., the portion of a sequence of a target molecule. In some aspects, the barcode sequence is used to identify a molecule of interest, a mutation in a molecule of interest, or the source of a molecule of interest, such as a cell-of-origin. In some aspects, the barcode sequence is less than 50 nucleotides.

As used herein, the term “unique” refers to a member of a set that is different from other members of the set. For example, a unique barcode sequence in library refers to a barcode that has a sequence that is not shared by other barcodes in the library. It should be understood that a “unique” barcode may exist in a population of cells in more than one copy after cells labelled with the barcodes begin to divide.

As used herein, the term “capture sequence” refers to a sequence on a molecule or construct that is recognized by an entity. In some aspects, the recognition allows the molecule or construct to be separated from a larger number of molecules or constructs. In some aspects, the entity can be a nucleic acid. In some aspects, the nucleic acid may be a primer that allows for reverse transcription of the construct.

As used herein, the term “scaffold sequence” refers to a sequence that is used to connect or link other sequences together (e.g., a barcode sequence and a capture sequence).

As used herein, the term “shield sequence” refers to a sequence at an end of a molecule (e.g., a nucleic acid sequence) that increases stability of said molecule. In some aspects, a shield sequence can be at the 5′ end of the molecule, the 3′ end of the molecule, or both. In some aspects, the shield sequence can be connected to the molecule (e.g., a nucleic acid sequence) by a linker (e.g. a scaffold sequence).

As used herein, the term “terminator sequence” refers to a sequence that signals the end of transcription.

As used herein, the term “5” refers to the 5′ end of a DNA or RNA sequence. As used herein, the term “3” refers to the 3′ end of a DNA or RNA sequence.

As used herein, the term “gene expression profile” refers to differential or altered gene expression that can be detected by changes in the detectable amount of gene expression (such as cDNA or mRNA) or by changes in the detectable amount of proteins expressed by those genes. A gene expression profile (also referred to as a fingerprint) can be linked to a tissue or cell type (such as ovarian cancer cell), to a particular stage of normal tissue growth or disease progression (such as advanced ovarian cancer), or to any other distinct or identifiable condition that influences gene expression in a predictable way. Gene expression profiles can include relative as well as absolute expression levels of specific genes, and can be viewed in the context of a test sample compared to a baseline or control sample profile (such as a sample from a subject who does not have ovarian cancer or normal endothelial cells). In one example, a gene expression profile in a subject is read on an array (such as a nucleic acid or protein array). For example, a gene expression profile is performed using a commercially available array such as a Human Genome U133 2.0 Plus Microarray from AFFYMETRIX® (AFFYMETRIX®, Santa Clara, Calif.).

As used herein, the term “CAR-T cell” includes cells engineered to express a Chimeric Antigen Receptor (CAR). CARs are typically artificial, recombinant polypeptides comprising at least (i) an extracellular domain that binds to a particular antigen, e.g., a tumor-specific antigen or a tumor-associated antigen, (ii) a transmembrane domain, and (iii) a primary signaling domain.

As used herein, the term “TCR-T cell” includes cells engineered to express a T cell receptor (TCR).

As used herein, the term “multiplexing” refers to the combination of multiple samples together to allow for simultaneous analysis. In some aspects, the multiplexing analysis is by RNA-sequencing. As used herein, the term “demultiplexing” refers to process by which analyzed data are assigned to their original samples based on an identifier. In some aspects, as the identifier is a barcode sequence.

As used herein, the term “vector” refers to a carrier or any tool that allows or facilitates the transfer of an entity from one environment to another. In some aspects, a vector is a replicon, such as a plasmid, phage, or cosmid, into which another DNA segment may be inserted so as to bring about the replication of the inserted segment. Generally, a vector is capable of replication when associated with the proper control elements. In some aspects, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. In some aspects, the vector is a “plasmid” which refers to a circular double stranded DNA loop into which additional DNA segments can be inserted, such as by standard molecular cloning techniques. In some aspects, the vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

As used herein, “promoter” refers to a DNA sequence capable of controlling the expression of a coding sequence or functional RNA. In some aspects, the promoter sequence includes proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

As used herein, “operably linked” refers to the sequential arrangement of the promoter polynucleotide according to the disclosure with a further oligo- or polynucleotide, resulting in promoter-driven transcription of said further polynucleotide.

As used herein, the term “viral vector” refers to a nucleic acid that includes at least one element of viral origin and includes elements sufficient for or permissive of packaging into a viral vector particle. The vector and/or particle can be utilized for the purpose of transferring DNA, RNA, or other nucleic acids into cells in vitro, ex vivo, or in vivo. Numerous forms of viral vectors are known. In some aspects, the delivery vector of the disclosure is a viral vector selected from the group consisting of an adeno-associated viral (AAV) vector, an adenoviral vector, a lentiviral vector, or a retroviral vector.

As used herein, a “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

As used herein, “nucleic acid”, in its broadest sense, refers to any compound and/or substance that is or can be incorporated into an oligonucleotide chain. In some aspects, a nucleic acid is a compound and/or substance that is or can be incorporated into an oligonucleotide chain via a phosphodiester linkage. As will be clear from context, in some aspects, “nucleic acid” refers to an individual nucleic acid residue (e.g., a nucleotide and/or nucleoside); in some aspects, “nucleic acid” refers to an oligonucleotide chain comprising individual nucleic acid residues. In some aspects, a “nucleic acid” is or comprises RNA; in some aspects, a “nucleic acid” is or comprises DNA. In some aspects, a nucleic acid is, comprises, or consists of one or more natural nucleic acid residues. In some aspects, a nucleic acid is, comprises, or consists of one or more nucleic acid analogs. In some aspects, a nucleic acid analog differs from a nucleic acid in that it does not utilize a phosphodiester backbone. For example, in some aspects, a nucleic acid is, comprises, or consists of one or more “peptide nucleic acids”, which are known in the art and have peptide bonds instead of phosphodiester bonds in the backbone, are considered within the scope of the present technology. Alternatively, or additionally, in some aspects, a nucleic acid has one or more phosphorothioate and/or 5′-N-phosphoramidite linkages rather than phosphodiester bonds. In some aspects, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some aspects, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof). In some aspects, a nucleic acid comprises one or more modified sugars (e.g., 2′-fluororibose, ribose, 2′-deoxyribose, arabinose, hexose or Locked Nucleic acids) as compared with those in commonly occurring natural nucleic acids. In some aspects, a nucleic acid has a nucleotide sequence that encodes a functional gene product such as an RNA or protein. In some aspects, a nucleic acid includes one or more introns. In some aspects, a nucleic acid may be a non-protein coding RNA product, such as a microRNA, a ribosomal RNA, or a CRISPR/Cas9 guide RNA. In some aspects, a nucleic acid serves a regulatory purpose in a genome. In some aspects, a nucleic acid does not arise from a genome. In some aspects, a nucleic acid includes intergenic sequences. In some aspects, a nucleic acid derives from an extrachromosomal element or a nonnuclear genome (mitochondrial, chloroplast etc.), In some aspects, nucleic acids are prepared by one or more of isolation from a natural source, enzymatic synthesis by polymerization based on a complementary template (in vivo or in vitro), reproduction in a recombinant cell or system, and chemical synthesis. In some aspects, a nucleic acid is at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some aspects, a nucleic acid is partly or wholly single stranded; in some aspects, a nucleic acid is partly or wholly double-stranded. In some aspects a nucleic acid has a nucleotide sequence comprising at least one element that encodes, or is the complement of a sequence that encodes, a polypeptide. In some aspects, a nucleic acid has enzymatic activity. In some aspects the nucleic acid serves a mechanical function, for example in a ribonucleoprotein complex or a transfer RNA. In some aspects a nucleic acid function as an aptamer. In some aspects a nucleic acid may be used for data storage. In some aspects a nucleic acid may be chemically synthesized in vitro.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

In the context of the invention, the term “treating” or “treatment”, as used herein, means reversing, alleviating, inhibiting the progress of, or preventing the disorder or condition to which such term applies, or one or more symptoms of such disorder or condition.

As used herein, the term “lineage tracing” refers to a set of methods or steps that allows the fate of individual cells and their progeny to be followed or analyzed. Lineage tracing allows the identification of all progeny of a single cell within a population of cells or within a data set comprising the sequences of a population of cells.

As used herein, the term “transcriptionally profiling” refers to the quantification of gene expression in cells or a population of cells at the RNA level.

### II. Barcoded RNA Constructs

In some aspects, provided herein is a barcoded RNA (also referred to herein as a barcoded RNA construct) comprising a first shield sequence at the 5′ end of the barcoded RNA, a barcode sequence, a scaffold sequence, a capture sequence, and a second shield sequence at the 3′ end of the barcoded RNA.

In some aspects, the barcoded RNA comprises a shield sequence (e.g., a first and/or second shield sequence). In some aspects, the shield sequence protects the barcoded RNA from endonucleases. In some aspects, the first shield sequence comprises at least one stem loop. In some aspects, the first shield sequence is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 10. In some aspects, the first shield sequence is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 10. In some aspects, the first shield sequence is between about 60% to about 100%, between about 70% to about 100%, between about 80% to about 100%, between about 85% to about 100%, between about 90% to about 100%, or between about 95% to about 100% identical to the nucleotide sequence set forth as SEQ ID NO: 10. In some aspects, the first shield sequence comprises the nucleotide sequence set forth as SEQ ID NO: 10. In some aspects, the first shield sequence is encoded by the nucleotide sequence set forth as SEQ ID NO: 1.

In some aspects, the first shield sequence is between 15 nucleotides to 50 nucleotides, between 15 nucleotides to 40 nucleotides, between 15 nucleotides to 35 nucleotides, between 15 nucleotides to 30 nucleotides, between 20 nucleotides to 30 nucleotides, between 25 nucleotides to 40 nucleotides, or between 25 nucleotides to 30 nucleotides long.

In some aspects, the first shield sequence comprises the first 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, or 50 nucleotides of the U6 non-coding small nuclear RNA. In some aspects, the first shield sequence comprises a γ-methyl phosphate cap.

In some aspects, the first shield sequence comprises at least the first 15 nucleotides, at least the first 16 nucleotides, at least the first 17 nucleotides, at least the first 18 nucleotides, at least the first 19 nucleotides, at least the first 20 nucleotides, at least the first 21 nucleotides, at least the first 22 nucleotides, at least the first 23 nucleotides, at least the first 24 nucleotides, at least the first 25 nucleotides, at least the first 26 nucleotides, at least the first 27 nucleotides, at least the first 28 nucleotides, at least the first 29 nucleotides, at least the first 30 nucleotides, at least the first 31 nucleotides, at least the first 32 nucleotides, at least the first 33 nucleotides, at least the first 34 nucleotides, at least the first 35 nucleotides, at least the first 36 nucleotides, at least the first 37 nucleotides, at least the first 38 nucleotides, at least the first 39 nucleotides, at least the first 40 nucleotides, at least the first 41 nucleotides, at least the first 42 nucleotides, at least the first 43 nucleotides, at least the first 44 nucleotides, at least the first 45 nucleotides, at least the first 46 nucleotides, at least the first 47 nucleotides, at least the first 48 nucleotides, at least the first 49 nucleotides, or at least the first 50 nucleotides of the U6 non-coding small nuclear RNA.

In some aspects, the first shield sequence is between the first 15 nucleotides to the first 50 nucleotides, between the first 15 nucleotides to the first 40 nucleotides, between the first 15 nucleotides to the first 35 nucleotides, between the first 15 nucleotides to the first 30 nucleotides, between the first 20 nucleotides to the first 30 nucleotides, between the first 25 nucleotides to the first 30 nucleotides, between the first 27 nucleotides to the first 50 nucleotides, between the first 27 nucleotides to the first 40 nucleotides, or between the first 20 nucleotides to the first 27 nucleotides of the U6 non-coding small nuclear RNA.

In some aspects, the second shield sequence comprises at least one stem loop. In some aspects, the second shield sequence is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 11. In some aspects, the second shield sequence is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 11. In some aspects, the second shield sequence is between about 60% to about 100%, between about 70% to about 100%, between about 80% to about 100%, or between about 90% to about 100% identical to the nucleotide sequence set forth as SEQ ID NO: 11. In some aspects, the second shield sequence comprises the nucleotide sequence set forth as SEQ ID NO: 11. In some aspects, the second shield sequence is encoded by the nucleotide sequence set forth as SEQ ID NO: 2.

In some aspects, the second shield sequence comprises an artificial sequence. In some aspects, the artificial sequence forms an artificial stem loop in the barcoded RNA. In some aspects the artificial stem loop protects the barcoded RNA from exonuclease degradation. In some aspects, the artificial stem loop protects the barcoded RNA from 3′ to 5′ exonuclease degradation.

In some aspects, the barcoded RNA further comprises a terminator sequence. In some aspects, the second shield sequence comprises an artificial stem loop. In some aspects, the second shield sequence comprises both an artificial stem loop and a terminator sequence. In some aspects, the second shield sequence comprises an artificial stem loop at the 5′ end of the second shield sequence and a terminator sequence at the 3′ end of the second shield sequence. In some aspects, the second shield sequence comprises an artificial stem loop at the 5′ end of the second shield sequence and a terminator sequence immediately following the second shield sequence.

In some aspects, the second shield sequence is at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, or at least 50 nucleotides long.

In some aspects, the second shield sequence is between 15 nucleotides to 50 nucleotides, between 15 nucleotides to 40 nucleotides, between 15 nucleotides to 30 nucleotides, between 15 nucleotides to 20 nucleotides, between 29 nucleotides to 50 nucleotides, between 29 nucleotides to 40 nucleotides, or between 20 nucleotides to 29 nucleotides long.

In some aspects, the barcode sequence is 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, or 50 nucleotides long. In some aspects, the barcode sequence is at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, or at least 50 nucleotides long.

In some aspects, the barcode sequence is between 2 nucleotides to 50 nucleotides, between 5 nucleotides to 50 nucleotides, between 8 nucleotides to 50 nucleotides, between 10 nucleotides to 50 nucleotides, between 15 nucleotides to 50 nucleotides, between 20 nucleotides to 50 nucleotides, between 25 nucleotides to 50 nucleotides, between 30 nucleotides to 50 nucleotides, between 35 nucleotides to 50 nucleotides, between 40 nucleotides to 50 nucleotides, between 45 nucleotides to 50 nucleotides, between 5 nucleotides to 45 nucleotides, between 5 nucleotides to 40 nucleotides, between 5 nucleotides to 35 nucleotides, between 5 nucleotides to 30 nucleotides, between 5 nucleotides to 25 nucleotides, between 5 nucleotides to 20 nucleotides, between 5 nucleotides to 15 nucleotides, between 5 nucleotides to 8 nucleotides, between 8 nucleotides to 45 nucleotides, between 8 nucleotides to 40 nucleotides, between 8 nucleotides to 35 nucleotides, between 8 nucleotides to 30 nucleotides, between 8 nucleotides to 25 nucleotides, between 8 nucleotides to 20 nucleotides, between 8 nucleotides to 15 nucleotides, or between 8 nucleotides to 10 nucleotides long.

In some aspects, the barcoded RNA comprises a scaffold sequence. In some aspects, the scaffold sequence comprises a single guide RNA (sgRNA). In some aspects, the sgRNA has been modified to avoid premature termination of Pol-III transcription. In some aspects, the sgRNA has been modified to delete a TTTT stretch within the stem-loop. In some aspects, the sgRNA comprises a protospacer. In some aspects, the protospacer is the barcode sequence.

In some aspects, the sgRNA comprises the nucleotide sequence set forth as SEQ ID NO: 13. In some aspects, the sgRNA is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 13. In some aspects, the sgRNA is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 13. In some aspects, the sgRNA comprises between about 60% to about 100%, between about 70% to about 100%, between about 80% to about 100%, or between about 90% to about 100% identical to the nucleotide sequence set forth as SEQ ID NO: 13. In some aspects, the sgRNA is encoded by the nucleotide sequence set forth as SEQ ID NO: 4.

In some aspects, the scaffold sequence comprises a three-way junction motif. In some aspects, the scaffold sequence comprises a four-way junction motif. In some aspects, the scaffold sequence comprises a five-way junction motif.

In some aspects, the scaffold sequence comprises a bacteriophage pRNA. In some aspects, the bacteriophage pRNA is phi29 (F29). In some aspects, the F29 pRNA contributes to high thermodynamic stability, highly efficient complex assembly, and/or resistance to denaturation. In some aspects, the F29 pRNA comprises a first arm and a second arm. In some aspects, an aptamer has been inserted into the first arm of the F29 pRNA. In some aspects, an aptamer has been inserted into the second arm of the F29 pRNA. In some aspects, an aptamer has been inserted into both the first arm and the second arm of the F29 pRNA. In some aspects, the F29 pRNA comprises the nucleotide sequence set forth as SEQ ID NO: 14. In some aspects, the F29 pRNA is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 14. In some aspects, the F29 pRNA is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 14. In some aspects, the F29 pRNA is between about 60% to about 100%, between about 70% to about 100%, between about 80% to about 100%, or between about 90% to about 100% identical to the nucleotide sequence set forth as SEQ ID NO: 14. In some aspects, the F29 pRNA is encoded by the nucleotide sequence set forth as SEQ ID NO: 5.

In some aspects, the bacteriophage pRNA comprises phi30 (F30). In some aspects, the F30 pRNA comprises a first arm and a second arm. In some aspects, an aptamer has been inserted into the first arm of the F30 pRNA. In some aspects, an aptamer has been inserted into the second arm of the F30 pRNA. In some aspects, an aptamer has been inserted into both the first arm and the second arm of the F30 pRNA. In some aspects, the F30 pRNA comprises the nucleotide sequence set forth as SEQ ID NO: 15. In some aspects, the F30 pRNA is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 15. In some aspects, the F30 pRNA is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 15. In some aspects, the F30 pRNA is between about 60% to about 100%, between about 70% to about 100%, between about 80% to about 100%, or between about 90% to about 100% identical to the nucleotide sequence set forth as SEQ ID NO: 15. In some aspects, the F30 pRNA is encoded by the nucleotide sequence set forth as SEQ ID NO: 6.

In some aspects, the barcoded RNA comprises a capture sequence. In some aspects, the capture sequence comprises the nucleotide sequence set forth as SEQ ID NO: 12. In some aspects, the capture sequence is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 12. In some aspects, the capture sequence is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 12. In some aspects, the capture sequence is between about 60% to about 100%, between about 70% to about 100%, between about 80% to about 100%, or between about 90% to about 100% identical to the nucleotide sequence set forth as SEQ ID NO: 12. In some aspects, the capture sequence is encoded by the nucleotide sequence set forth as SEQ ID NO: 3.

In some aspects, the capture sequence is recognized by an entity. In some aspects, the recognition allows the barcoded RNA to be separated from non-barcoded RNA. In some aspects, the entity can be a nucleic acid. In some aspects, the nucleic acid is a primer. In some aspects, the primer allows for reverse transcription of the construct. In some aspects, the capture sequence is captured directly by gel beads (e.g., Chromium Single Cell 3′ v3 Gel Beads). In some aspects, the nucleic acid is an oligonucleotide that hybridizes to the capture sequence. In some aspects, the oligonucleotide comprises a label. In some aspects, the label is a radioactive phosphate, biotin, a fluorophore, chemical tag, antibody, or an enzyme. In some aspects, the label is biotin. In some aspects, the biotin may be captured by streptavidin. In some aspects, the streptavidin is conjugated to a bead.

In some aspects, the barcoded RNA further comprises a RNA aptamer. In some aspects, the RNA aptamer is a fluorescent RNA aptamer. In some aspects, the fluorescent RNA aptamer is a Broccoli RNA aptamer, a Spinach RNA aptamer, a Pepper RNA aptamer, a Mango II RNA aptamer, a malachite green aptamer. In some aspects, the fluorescent RNA aptamer is a Broccoli RNA aptamer. In some aspects, the Broccoli RNA aptamer sequence has been optimized.

In some aspects, the barcoded RNA comprises the nucleotide sequence set forth as SEQ ID NO: 16. In some aspects, the barcoded RNA comprises a sequence at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 16. In some aspects, the barcoded RNA comprises a sequence about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 16. In some aspects, the barcoded RNA is between about 60% to about 100%, between about 70% to about 100%, between about 80% to about 100%, between about 85% to about 100%, between about 90% to about 95%, or between about 95% to about 100% identical to the nucleotide sequence set forth as SEQ ID NO: 16. In some aspects, the barcoded RNA is encoded by the nucleotide sequence set forth as SEQ ID NO: 7.

In some aspects, the barcoded RNA comprises the nucleotide sequence set forth as SEQ ID NO: 17.

In some aspects, the barcoded RNA comprises the nucleotide sequence set forth as SEQ ID NO: 18 In some aspects, the barcoded RNA comprises a sequence at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 18. In some aspects, the barcoded RNA comprises a sequence about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 18. In some aspects, the barcoded RNA comprises a sequence between about 60% to about 100%, between about 70% to about 100%, between about 80% to about 100%, between about 85% to about 100%, between about 90% to about 100%, or between about 95% to about 100% identical to the nucleotide sequence set forth as SEQ ID NO: 18. In some aspects, the barcoded RNA is encoded by the nucleotide sequence set forth as SEQ ID NO: 9.

In some aspects, the first shield sequence, the barcode sequence, the scaffold sequence, the capture sequence, and the second shield sequence comprise a 5′ nucleotide and a 3′ nucleotide. In some aspects, the 3′ nucleotide of the first shield sequence is next to the 5′ nucleotide of the barcode sequence. In some aspects, the 3′ end of the barcode sequence is next to the 5′ end of the scaffold sequence. In some aspects, the capture sequence forms a secondary structure in the middle of the scaffold sequence. In some aspects, the 3′ end of the scaffold sequence is next to the 5′ end of the second shield sequence.

In some aspects, the 5′ end of the capture sequence is next to nucleotide 66 of the scaffold sequence corresponding to SEQ ID NO: 13. In some aspects, the 3′ end of the capture sequence is next to nucleotide 67 of the scaffold sequence corresponding to SEQ ID NO: 13. In some aspects, the 5′ end of the capture sequence is next to nucleotide 35 of the scaffold sequence corresponding to SEQ ID NO:14. In some aspects, the 3′ end of the capture sequence is next to nucleotide 36 of the scaffold sequence corresponding to SEQ ID NO: 14. In some aspects, the 5′ end of the capture sequence is next to nucleotide 35 of the scaffold sequence corresponding to SEQ ID NO:15. In some aspects, the 3′ end of the capture sequence is next to nucleotide 36 of the scaffold sequence corresponding to SEQ ID NO: 15.

In some aspects, the barcoded RNA is administered in the absence of a Cas protein (e.g., a Cas9). In some aspects, the barcoded RNA is stable in the absence of a Cas9 protein.

In some aspects, the barcoded RNA comprises a first arm and a second arm, wherein the second arm comprises the barcode sequence.

### III. Methods of Using the Barcoded RNA

Certain aspects of the disclosure are directed to small RNAs engineered as sample barcodes for multiplex labeling in single-cell RNA sequencing. In some aspects, using Shielded Small Nucleotide-seq (SSN-seq) for intracellular barcoding cells allows for multiplexed single-cell RNA sequencing. In some aspects, the engineered small RNAs used for multiplex labeling in single-cell RNA sequencing comprise a first shield sequence at the 5′ end of the barcoded RNA, a barcode sequence, a scaffold sequence, a capture sequence, and a second shield sequence at the 3′ end of the barcoded RNA.

In some aspects, the scaffold sequence is a sgRNA. In some aspects, the scaffold sequence is a bacteriophage pRNA. In some aspects, the bacteriophage pRNA is F29. In some aspects, the bacteriophage pRNA is F30. In some aspects, the scaffold sequence can be used to link a sample barcode to a capture sequence, e.g., further including an anti-degradation motif. In some aspects, SSN-seq using multiple cell types including human primary T cells is provided. In some aspects, SSN-seq achieves efficient sample assignments, promoting cell profiling in a cost-effective “label-pool-demultiplex” way. Methods of using the barcoded RNA disclosed herein for multiplex labeling in single-cell RNA sequencing are disclosed herein.

In some aspects, provided herein are methods of performing single-cell RNA sequencing using a barcoded RNA disclosed herein. In some aspects, the methods of performing single-cell RNA sequencing comprise introducing a barcoded RNA library to a population of cells and performing single-cell RNA sequencing on the population of cells.

In some aspects, the barcoded RNA library comprises a plurality of barcoded RNA constructs. In some aspects, the barcoded RNA construct comprises a first shield sequence at the 5′ end of the barcoded RNA, a unique barcode sequence, a scaffold sequence, a capture sequence, and a second shield sequence at the 3′ end of the barcoded RNA. In some aspects, the cells can be identified by the unique barcode sequence. In some aspects, an individual cell has a gene expression profile.

In some aspects, a subpopulation of cells can be identified when a plurality of cells comprise the same barcode sequence.

In some aspects, the single-cell RNA sequencing is performed with a single-cell sequencing platform (e.g., 10× Genomics® Chromium®). In some aspects, the single-cell RNA sequencing is performed following a protocol (e.g., 10× Genomics® Chromium® Single Cell 3′ workflow).

In some aspects, the barcoded RNA library is introduced to the population of cells prior to an in vivo experiment. In some aspects, the barcoded RNA library is introduced to the population of cells prior to an in vitro experiment.

In some aspects, the barcoded RNA library comprises a barcoded RNA comprising a sgRNA scaffold sequence. In some aspects, the barcoded RNA library comprises a barcoded RNA comprising a F29 scaffold sequence. In some aspects, the barcoded RNA library comprises a barcoded RNA comprising a F30 scaffold sequence. In some aspects, the barcoded RNA library comprises barcoded RNAs comprising a sgRNA scaffold sequence, a F29 scaffold sequence, a F30 scaffold sequence, or any combination thereof.

In some aspects, a first barcoded RNA construct is introduced to a first population of cells, while a second barcoded RNA construct is introduced to a second population of cells. In some aspects, the first population of cells and second population of cells are pooled together after they have been labelled by the either the first barcoded RNA constructs or the second barcoded RNA construct, which allows for pooling of cells prior to an experiment. It should be appreciated that the number of populations that may be pooled together is determined by the number of unique barcode sequences available. As an example, in some aspects, for a barcode sequence that is 3 nucleotides in length, there are 43 or 64 different barcodes that may be used to uniquely label 64 different populations of cells. If the barcode sequence was 10 nucleotides in length, there are 410 or 1,048,576 different barcodes that may be used to uniquely label 1,048,576 different populations of cells.

In some aspects, the barcode sequence in the methods described herein is 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, or 50 nucleotides long. In some aspects, the barcode sequence is at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, or at least 50 nucleotides long.

In some aspects, the barcode sequence in the methods described herein is between 2 nucleotides to 50 nucleotides, between 5 nucleotides to 50 nucleotides, between 8 nucleotides to 50 nucleotides, between 10 nucleotides to 50 nucleotides, between 15 nucleotides to 50 nucleotides, between 20 nucleotides to 50 nucleotides, between 25 nucleotides to 50 nucleotides, between 30 nucleotides to 50 nucleotides, between 35 nucleotides to 50 nucleotides, between 40 nucleotides to 50 nucleotides, between 45 nucleotides to 50 nucleotides, between 5 nucleotides to 45 nucleotides, between 5 nucleotides to 40 nucleotides, between 5 nucleotides to 35 nucleotides, between 5 nucleotides to 30 nucleotides, between 5 nucleotides to 25 nucleotides, between 5 nucleotides to 20 nucleotides, between 5 nucleotides to 15 nucleotides, between 5 nucleotides to 8 nucleotides, between 8 nucleotides to 45 nucleotides, between 8 nucleotides to 40 nucleotides, between 8 nucleotides to 35 nucleotides, between 8 nucleotides to 30 nucleotides, between 8 nucleotides to 25 nucleotides, between 8 nucleotides to 20 nucleotides, between 8 nucleotides to 15 nucleotides, or between 8 nucleotides to 10 nucleotides long.

In some aspects, the barcoded RNA library is introduced to the population of cells by a viral vector. In some aspects, the viral vector is an adeno-associated viral (AAV) vector, an adenoviral vector, a lentiviral vector, or a retroviral vector. In some aspects, the viral vector is administered to an animal model. In some aspects, the viral vector is administered by standard routes including, but not limited to, pulmonary, intranasal, oral, inhalation, parenteral such as intravenous (IV), topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection.

In some aspects, the viral vector comprises at least a first barcoded RNA and a second barcoded RNA. In some aspects, the first barcoded RNA and the second barcoded RNA are the same. In some aspects, the first barcoded RNA and the second barcoded RNA are different. In some aspects, the first barcoded RNA comprises a sgRNA scaffold. In some aspects, the first barcoded RNA comprises a F29 scaffold. In some aspects, the first barcoded RNA comprises a F30 scaffold. In some aspects, the second barcoded RNA comprises a sgRNA scaffold. In some aspects, the second barcoded RNA comprises a F29 scaffold. In some aspects, the second barcoded RNA comprises a F30 scaffold. In some aspects, the first barcoded RNA comprises a F30 scaffold and the second barcoded RNA comprises a sgRNA scaffold. In some aspects, the first barcoded RNA comprises a F29 scaffold and the second barcoded RNA comprises a sgRNA scaffold. In some aspects, the first barcoded RNA comprises a F30 scaffold and the second barcoded RNA comprises a F29 scaffold.

In some aspects, the viral vector comprises a unique oncogene such that the unique barcoded RNA of the barcoded RNA library is associated with a unique oncogene. In some aspects, the unique barcoded RNA and unique oncogene are introduced to an organ. In some aspects, the organ can be a kidney, a liver, a pancreas, a heart, a lung, skin, small intestine, an endothelial tissue, a vascular tissue, an eye, a stomach, a thymus, bone, bone marrow, cornea, a heart valve, an islet of Langerhans, or a tendon. In some aspects, the administration to the organ results in the growth of a tumor. In some aspects, a sample of the tumor may be isolated and analyzed by single-cell RNA sequencing. In some aspects, the entire tumor is dissected and analyzed. In some aspects, a biopsy of the tumor obtains a sample to be analyzed. In some aspects, the data from the single-cell RNA sequencing is demultiplexed through identification of the barcoded RNAs in the labelled cell. In some aspects, a high frequency of a barcoded RNA in the analyzed tumor cells indicates strong oncogene activity by the oncogene associated with the barcoded RNA. In some aspects, a low frequency of a barcoded RNA in the analyzed tumor cells indicates weak oncogene activity by the oncogene associated with the barcoded RNA.

In some aspects, the population of cells to be labelled are CAR-T cells. In some aspects, the CAR-T cells comprise different genetic edits. In some aspects, the CAR-T cells are administered to an animal model. In some aspects, the animal model has a tumor. In some aspects, the tumor is isolated. In some aspects, the entire tumor is isolated by dissection. In some aspects, a biopsy of the tumor obtains a sample to be analyzed. In some aspects, the isolated tumor or biopsy sample is analyzed by single-cell RNA sequencing. In some aspects, the data from the single-cell RNA sequencing is demultiplexed through identification of the unique barcoded RNAs in the labelled cell. In some aspects, the phenotype of the genetic edits may be determined. In some aspects, the phenotype may relate to strong infiltrating activity by the CAR-T cell. In some aspects, CAR-T cells with a strong infiltrating activity phenotype may be to be administered treat cancer in a patient. In some aspects, the cancer is leukemia, lymphoma, myeloma, bladder cancer, breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas and/or ovarian cancer.

In some aspects, the population of cells to be labelled comprise tumor infiltrating immune cells. In some aspects, the tumor infiltrating immune cells comprise a unique B cell receptor signature. In some aspects, the tumor infiltrating immune cells comprise a unique T cell receptor signature. In some aspects, the unique B cell receptor signature is indicative of a strong response to tumor cells. In some aspects, the unique T cell receptor signature is indicative of a strong response to tumor cells. In some aspects, the labelled cells are administered to an animal model. In some aspects, the animal model is a mouse, a hamster, a rabbit, a nonhuman primate, a guinea pig, a rat, a zebrafish, a pig, a sheep, a cat, or a dog. In some aspects, the animal model has a tumor. In some aspects, the tumor is isolated. In some aspects, the entire tumor is isolated by dissection. In some aspects, a biopsy of the tumor obtains a sample to be analyzed. In some aspects, the isolated tumor or biopsy is analyzed by single-cell RNA sequencing. In some aspects, the data from the single-cell RNA sequencing is demultiplexed through identification of the unique barcoded RNAs in the labelled cell. In some aspects, tumor infiltrating immune cells may be selected for therapeutic administration based on the number of tumor infiltrating immune cells present within the tumor sample, where a high number of related tumor infiltrating immune cells as indicated by the same unique barcoded RNA indicates strong tumor infiltrating activity. In some aspects, a low number of related tumor infiltrating immune cells as indicated by the same unique barcoded RNA indicates weak tumor infiltrating activity. In some aspects, tumor infiltrating immune cells identified as having strong tumor infiltrating activity are administered to a patient in need thereof. In some aspects, the patient in need thereof is suffering from a cancer such as breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas and/or ovarian cancer.

In some aspects, the population of cells are cancer cells. In some aspects, the unique barcoded RNA of the barcoded RNA library is associated with a unique gene and administered to the cancer cells. In some aspects, the unique gene can be an oncogene, a tumor suppressor, or a gene with an unknown function.

In some aspects, the population of cancer cells are introduced into an animal model. In some aspects, the animal model is a mouse, a hamster, a rabbit, a nonhuman primate, a guinea pig, a rat, a zebrafish, a pig, a sheep, a cat, or a dog. In some aspects, the population of cancer cells develops into a tumor in the animal model. In some aspects, the entire tumor is isolated by dissection. In some aspects, a biopsy of the tumor obtains a sample to be analyzed. In some aspects, the isolated tumor or biopsy sample is analyzed by single-cell RNA sequencing into a single-cell RNA sequencing dataset. In some aspects, the data from the single-cell RNA sequencing is demultiplexed through identification of the barcoded RNAs in the labelled cell.

In some aspects, a high number of a unique RNA barcode within a single-cell RNA sequencing dataset may indicate that the unique gene that is associated with the unique barcode has oncogenic activity. In some aspects, a low number of a unique RNA barcode within a single-cell RNA sequencing dataset may indicate that the unique gene that is associated with the unique barcode has tumor suppressor activity.

In some aspects, the population of cancer cells are breast cancer cells, brain cancer cells, lung cancer cells, liver cancer cells, stomach cancer cells, spleen cancer cells, colon cancer cells, renal cancer cells, pancreatic cancer cells, prostate cancer cells, uterine cancer cells, skin cancer cells, head cancer cells, neck cancer cells, sarcoma cells, neuroblastoma cells or ovarian cancer cells.

In some aspects, the tumor is allowed to develop in the animal for 1 week, 2 weeks, 3 weeks, 4 weeks, 5 weeks, 6 weeks, 7 weeks, 8 weeks, 9 weeks, 10 weeks, 15 weeks, 20 weeks, 25 weeks, 30 weeks, 35 weeks, 40 weeks, or 52 weeks prior to analysis. In some aspects, the tumor is allowed to develop in the animal model for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 15 weeks, at least 20 weeks, at least 25 weeks, at least 30 weeks, at least 35 weeks, at least 40 weeks, or at least 52 weeks prior to analysis. In some aspects, the tumor is allowed to develop in the animal model for 1 to 5 weeks, 1 to 10 weeks, 1 to 20 weeks, 1 to 30 weeks, 1 to 40 weeks, 1 to 50 weeks, 5 to 10 weeks, 5 to 20 weeks, 5 to 30 weeks, 5 to 40 weeks, 5 to 50 weeks, 10 to 20 weeks, 10 to 30 weeks, 10 to 40 weeks, or 10 to 50 weeks prior to analysis. In some aspects, the analysis begins with dissection of the entire tumor. In some aspects, the analysis begins with biopsy of a portion of the tumor.

In some aspects, the first shield sequence in the methods described herein comprises the nucleotide sequence set forth as SEQ ID NO: 10. In some aspects, the first shield sequence is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 10. In some aspects, the first shield sequence is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 10. In some aspects, the first shield sequence is between about 60% to about 100%, between about 70% to about 100%, between about 80% to about 100%, or between about 90% to about 100% identical to the nucleotide sequence set forth as SEQ ID NO: 10. In some aspects, the first shield sequence is encoded by the nucleotide sequence set forth as SEQ ID NO: 1.

In some aspects, the first shield sequence in the methods described herein is the first 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, or 50 nucleotides of the U6 non-coding small nuclear RNA. In some aspects, the first shield sequence comprises a γ-methyl phosphate cap.

In some aspects, the first shield sequence in the methods described herein is at least the first 15 nucleotides, at least the first 16 nucleotides, at least the first 17 nucleotides, at least the first 18 nucleotides, at least the first 19 nucleotides, at least the first 20 nucleotides, at least the first 21 nucleotides, at least the first 22 nucleotides, at least the first 23 nucleotides, at least the first 24 nucleotides, at least the first 25 nucleotides, at least the first 26 nucleotides, at least the first 27 nucleotides, at least the first 28 nucleotides, at least the first 29 nucleotides, at least the first 30 nucleotides, at least the first 31 nucleotides, at least the first 32 nucleotides, at least the first 33 nucleotides, at least the first 34 nucleotides, at least the first 35 nucleotides, at least the first 36 nucleotides, at least the first 37 nucleotides, at least the first 38 nucleotides, at least the first 39 nucleotides, at least the first 40 nucleotides, at least the first 41 nucleotides, at least the first 42 nucleotides, at least the first 43 nucleotides, at least the first 44 nucleotides, at least the first 45 nucleotides, at least the first 46 nucleotides, at least the first 47 nucleotides, at least the first 48 nucleotides, at least the first 49 nucleotides, or at least the first 50 nucleotides of the U6 non-coding small nuclear RNA.

In some aspects, the first shield sequence in the methods described herein is between the first 15 nucleotides to the first 50 nucleotides, between the first 15 nucleotides to the first 40 nucleotides, between the first 15 nucleotides to the first 30 nucleotides, between the first 15 nucleotides to the first 20 nucleotides, between the first 27 nucleotides to the first 50 nucleotides, between the first 27 nucleotides to the first 40 nucleotides, or between the first 20 nucleotides to the first 27 nucleotides of the U6 non-coding small nuclear RNA.

In some aspects, the second shield sequence in the methods described herein comprises the nucleotide sequence set forth as SEQ ID NO: 11. In some aspects, the second shield sequence is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 11. In some aspects, the first shield sequence is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 11. In some aspects, the first shield sequence is between about 60% to about 100%, between about 70% to about 100%, between about 80% to about 100%, or between about 90% to about 100% identical to the nucleotide sequence set forth as SEQ ID NO: 11. In some aspects, the second shield sequence is encoded by the nucleotide sequence set forth as SEQ ID NO: 2

In some aspects, the second shield sequence in the methods described herein comprises an artificial sequence. In some aspects, the artificial sequence forms an artificial stem loop in the barcoded RNA. In some aspects the artificial stem loop protects the barcoded RNA from exonuclease degradation. In some aspects, the artificial stem loop protects the barcoded RNA from 3′ to 5′ exonuclease degradation.

In some aspects, the barcoded RNA in the methods described herein further comprises a terminator sequence. In some aspects, the second shield sequence comprises an artificial stem loop. In some aspects, the second shield sequence comprises both an artificial stem loop and a terminator sequence. In some aspects, the second shield sequence comprises an artificial stem loop at the 5′ end of the second shield sequence and a terminator sequence at the 3′ end of the second shield sequence. In some aspects, the second shield sequence comprises an artificial stem loop at the 5′ end of the second shield sequence and a terminator sequence immediately following the second shield sequence.

In some aspects, the second shield sequence in the methods described herein is at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, or at least 50 nucleotides long.

In some aspects, the second shield sequence in the methods described herein is between 15 nucleotides to 50 nucleotides, between 15 nucleotides to 40 nucleotides, between 15 nucleotides to 30 nucleotides, between 15 nucleotides to 20 nucleotides, between 29 nucleotides to 50 nucleotides, between 29 nucleotides to 40 nucleotides, or between 20 nucleotides to 29 nucleotides long.

In some aspects, the scaffold sequence in the methods described herein is a single guide RNA (sgRNA). In some aspects, the sgRNA has been modified to avoid premature termination of Pol-III transcription. In some aspects, the sgRNA has been modified to delete a TTTT stretch within the stem-loop. In some aspects, the sgRNA comprises a protospacer. In some aspects the protospacer is the barcode sequence. In some aspects, the sgRNA comprises the nucleotide sequence set forth as SEQ ID NO: 13. In some aspects, the sgRNA is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 13. In some aspects, the sgRNA is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 13. In some aspects, the sgRNA is between about 60% to about 100%, between about 70% to about 100%, between about 80% to about 100%, or between about 90% to about 100% identical to the nucleotide sequence set forth as SEQ ID NO: 13. In some aspects, the sgRNA is encoded by the nucleotide sequence set forth as SEQ ID NO: 4.

In some aspects, the scaffold sequence in the methods described herein comprises a three-way junction motif. In some aspects, the scaffold sequence comprises a four-way junction motif. In some aspects, the scaffold sequence comprises a five-way junction motif. In some aspects, the scaffold sequence in the methods described herein is a bacteriophage pRNA. In some aspects, the bacteriophage pRNA is phi29 (F29). In some aspects, the F29 pRNA contributes to high thermodynamic stability, highly efficient complex assembly, and/or resistance to denaturation. In some aspects, the F29 pRNA comprises a first arm and a second arm. In some aspects, an aptamer has been inserted into the first arm of the F29 pRNA. In some aspects, an aptamer has been inserted into the second arm of the F29 pRNA. In some aspects, an aptamer has been inserted into both the first arm and the second arm of the F29 pRNA. In some aspects, the F29 pRNA comprises the nucleotide sequence set forth as SEQ ID NO: 14. In some aspects, the F29 pRNA is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 14. In some aspects, the F29 pRNA is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 14. In some aspects, the F29 pRNA is between about 60% to about 100%, between about 70% to about 100%, between about 80% to about 100%, or between about 90% to about 100% identical to the nucleotide sequence set forth as SEQ ID NO: 14. In some aspects, the F29 pRNA is encoded by the nucleotide sequence set forth as SEQ ID NO: 5.

In some aspects, the bacteriophage pRNA in the methods described herein is phi30 (F30). In some aspects, the F30 pRNA comprises a first arm and a second arm. In some aspects, an aptamer has been inserted into the first arm of the F30 pRNA. In some aspects, an aptamer has been inserted into the second arm of the F30 pRNA. In some aspects, an aptamer has been inserted into both the first arm and the second arm of the F30 pRNA. In some aspects, the F30 pRNA comprises the nucleotide sequence set forth as SEQ ID NO: 15. In some aspects, the F30 pRNA is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 15. In some aspects, the F30 pRNA is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 15. In some aspects, the F30 pRNA is between about 60% to about 100%, between about 70% to about 100%, between about 80% to about 100%, or between about 90% to about 100% identical to the nucleotide sequence set forth as SEQ ID NO: 15. In some aspects, the F30 pRNA is encoded by the nucleotide sequence set forth as SEQ ID NO: 6.

In some aspects, the capture sequence in the methods described herein comprises the nucleotide sequence set forth as SEQ ID NO: 12. In some aspects, the capture sequence is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 12. In some aspects, the capture sequence is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 12. In some aspects, the capture sequence is between about 60% to about 100%, between about 70% to about 100%, between about 80% to about 100%, or between about 90% to about 100% identical to the nucleotide sequence set forth as SEQ ID NO: 12. In some aspects, the capture sequence is encoded by the nucleotide sequence set forth as SEQ ID NO: 3.

In some aspects, the capture sequence in the methods described herein is recognized by an entity. In some aspects, the recognition allows the barcoded RNA to be separated from non-barcoded RNA. In some aspects, the entity can be a nucleic acid. In some aspects, the nucleic acid is a primer. In some aspects, the primer allows for reverse transcription of the construct. In some aspects, the nucleic acid is an oligonucleotide that hybridizes to the capture sequence. In some aspects, the oligonucleotide comprises a label. In some aspects, the label is a radioactive phosphate, biotin, a fluorophore, chemical tag, antibody, or an enzyme. In some aspects, the label is biotin. In some aspects, the biotin may be captured by streptavidin. In some aspects, the streptavidin is conjugated to a bead.

In some aspects, the barcoded RNA in the methods described herein further comprises a RNA aptamer. In some aspects, the RNA aptamer is a fluorescent RNA aptamer. In some aspects, the fluorescent RNA aptamer is a Broccoli RNA aptamer, a Spinach RNA aptamer, a Pepper RNA aptamer, a Mango II RNA aptamer, a malachite green aptamer. In some aspects, the fluorescent RNA aptamer is a Broccoli RNA aptamer. In some aspects, the Broccoli RNA aptamer sequence has been optimized.

In some aspects, the barcoded RNA in the methods described herein comprises the nucleotide sequence set forth as SEQ ID NO: 16. In some aspects, the barcoded RNA is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 16. In some aspects, the barcoded RNA is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 16. In some aspects, the barcoded RNA is between about 60% to about 100%, between about 70% to about 100%, between about 80% to about 100%, or between about 90% to about 100% identical to the nucleotide sequence set forth as SEQ ID NO: 16. In some aspects, the barcoded RNA is encoded by the nucleotide sequence set forth as SEQ ID NO: 7.

In some aspects, the barcoded RNA in the methods described herein comprises the nucleotide sequence set forth as SEQ ID NO: 17. In some aspects, the barcoded RNA is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 17. In some aspects, the barcoded RNA is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 17. In some aspects, the barcoded RNA is between about 60% to about 100%, between about 70% to about 100%, between about 80% to about 100%, or between about 90% to about 100% identical to the nucleotide sequence set forth as SEQ ID NO: 17. In some aspects, the barcoded RNA is encoded by the nucleotide sequence set forth as SEQ ID NO: 8.

In some aspects, the barcoded RNA in the methods described herein comprises the nucleotide sequence set forth as SEQ ID NO: 18. In some aspects, the barcoded RNA is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 18. In some aspects, the barcoded RNA is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 18. In some aspects, the barcoded RNA is between about 60% to about 100%, between about 70% to about 100%, between about 80% to about 100%, or between about 90% to about 100% identical to the nucleotide sequence set forth as SEQ ID NO: 18. In some aspects, the barcoded RNA is encoded by the nucleotide sequence set forth as SEQ ID NO: 9.

Recent advances in CRISPR-screen using single-cell RNA sequencing demonstrates that it is possible to directly capture the single-guide RNA (sgRNA) to serve as a barcode corresponding to the perturbation. Specifically, the sgRNA scaffold can be modified to harbor a capture sequence, which enables separated capturing from poly-adenylated mRNAs. For example, this allows profiling gene expression together with CRISPR-mediated phenotypes on the same cells.

In some aspects, provided herein are methods of detecting a gene expression profile of a Chimeric Antigen Receptor T cell (CAR-T cell). In some aspects, the method of detecting a gene expression profile of a CAR-T cell comprises: (a) transducing a plurality of T cells with (i) a Chimeric Antigen Receptor (CAR) and (ii) at least one barcoded RNA construct to form a population of CAR-T cells, (b) subjecting the population of CAR-T cells to a test condition, (c) collecting the population of CAR-T cells after the test condition, (d) pooling the population of CAR-T cells and (e) performing single-cell RNA sequencing to determine a gene expression profile the barcoded CAR-T cells in the population. In some aspects, the barcoded RNA construct comprises a 5′ shield sequence, a unique barcode sequence, a scaffold sequence, a capture sequence, and a 3′ shield sequence. In some aspects, the unique barcode sequence allows for demultiplexing of the population of CAR-T cells.

In some aspects, the method further comprises identifying a CAR-T cell with a desired gene expression profile by the CAR-T cell's barcode. In some aspects, the sequence of the CAR in the CAR-T cell with the desired gene expression profile is then used to develop additional CAR-T cells that are used to treat a patient.

In some aspects, the test condition is injection into a tumor in an animal model. In some aspects, the animal model is a mammal. In some aspects, the animal model is a mouse, a hamster, a rabbit, a nonhuman primate, a guinea pig, a rat, a zebrafish, a pig, a sheep, a cat, or a dog.

In some aspects, the gene expression profile displays genes involved in T cell activation. In some aspects, the genes involved in T cell activation include CD69, CD25, CD71, CD134, and/or CD137. In some aspects, the gene expression profile displays genes involved in T cell exhaustion. In some aspects, the genes involved in T cell exhaustion include PD-1, LAG-3, Tim-3, TIGIT, CTLA-4 and/or CD39. In some aspects, the gene expression profile displays genes involved in apoptosis. In some aspects, the genes involved in apoptosis include CD95, CD261, CD262, CD120a, TNF-R2, CD266, BCL-2, CASP3, CASP7, CASP8, and/or CASP9.

In some aspects, the T cells are transduced with a viral vector. In some aspects, the viral vector is an adeno-associated viral (AAV) vector, an adenoviral vector, a lentiviral vector, or a retroviral vector.

In some aspects, provided herein are methods of selecting a tumor infiltrating immune cell from a patient. In some aspects, the method of selecting a tumor infiltrating immune cell from a patient comprises isolating tumor infiltrating immune cells from a patient, introducing a barcoded RNA construct to the tumor infiltrating immune cell, challenging the tumor infiltrating immune cells with cancer cells, collecting the tumor infiltrating immune cells after the challenge, pooling the population of tumor infiltrating immune cells, performing single-cell RNA sequencing to determine a gene expression profile for the tumor infiltrating immune cell, and selecting a tumor infiltrating immune cell with the gene expression profile desired for treatment of the patient. In some aspects, the tumor infiltrating immune cell has a unique B cell receptor signature. In some aspects, the tumor infiltrating immune cell has a unique T cell receptor signature. In some aspects, the gene expression profile is indicative of increased activity towards tumor cells as compared to the average activity of a population of immune cells.

In some aspects, the barcoded RNA constructs comprise a 5′ shield sequence, a unique barcode sequence, a scaffold sequence, a capture sequence, and a 3′ shield sequence. In some aspects, the unique barcode sequence allows for demultiplexing of the population of CAR-T cells.

In some aspects, the barcoded RNA construct is introduced to the tumor infiltrating immune cell by a viral vector. In some aspects, the viral vector is an adeno-associated viral (AAV) vector, an adenoviral vector, a lentiviral vector, or a retroviral vector. In some aspects, the method further comprises administrating the selected tumor infiltrating immune cells to a patient.

In some aspects, provided herein are methods for analyzing tumor development. In some aspects, the method for analyzing tumor development comprises introducing at least one barcoded RNA construct to a population of cancer cells to form a sample population, injecting the sample population into an animal model, allowing a tumor to develop in the animal model, isolating the tumor from the animal model, performing single-cell RNA sequencing on cells in the tumor. In some aspects, the barcoded RNA construct comprises a 5′ shield sequence, a unique barcode sequence, a scaffold sequence, a capture sequence, and a 3′ shield sequence. In some aspects, the unique barcode sequence allows for demultiplexing of the sample.

In some aspects, the at least one barcoded RNA construct is introduced to the population of cancer cells by a viral vector. In some aspects, the viral vector is an adeno-associated viral (AAV) vector, an adenoviral vector, a lentiviral vector, or a retroviral vector.

In some aspects, the tumor is derived from a cancer. In some aspects, the cancer is a breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas and ovarian cancer.

In some aspects, provided herein are methods for analyzing oncogenes. In some aspects, the method for analyzing oncogenes comprises introducing a viral vector to an animal model, allowing a tumor to develop in the animal model, isolating the tumor from the animal model, performing single-cell RNA sequencing on cells in the tumor. In some aspects, the viral vector comprises a unique oncogene and a barcoded RNA construct. In some aspects, the barcoded RNA construct comprises a 5′ shield sequence, a unique barcode sequence, a scaffold sequence, a capture sequence, and a 3′ shield sequence. In some aspects, the unique barcode sequence allows for demultiplexing of the sample.

In some aspects, the oncogenes are selected from ABL1, ABL2, ACVR1, AKT1, AKT2, ALK, ATFL, BCL11A, BCL2, BCL6, BCR, BLC3, BRAF, CARD11, CBLB, CBLC, CCND1, CCND2, CCND3, CD79B, CDH1, CDK4, CDX2, CHD4, CNBD1, COL5A1, CTNNB1, CUL1, CYSLTR2, DACH1, DDB2, DDIT3, DDX6, DEK, DMD, EEF1A1, EGFR, EIF1AX, ELK4, EP300, EPAS1, ERBB2, ERBB3, ERBB4, ERCC2, ETV4, ETV6, EVIL EWSR1, FAM46D, FBXW7, FEV, FGFR1, FGFR1OP, FGFR2, FGFR3, FLT3, FOXA1, FUS, GNA11, GNA13, GNAQ, GNAS, GOLGA5, GTF2I, HMGA1, HMGA2, HRAS, IDH1, IDH2, IRF4, JUN, KEAP1, KIT, KLF5, KRAS, LCK, LMO2, MAF, MAFB, MAML2, MAP2K1, MAPK1, MAX, MDM2, MED12, MET, MITF, MLL, MPL, MTOR, MYB, MYC, MYCL1, MYCN, MYD88, MYH9, NCOA4, NFE2L2, NFKB2, NPM1, NRAS, NTRK1, NUP214, PAX8, PCBP1, PDGFB, PIK3CA, PIM1, PLAGI, PLCB4, PLCG1, POLRMT, PPARG, PPP2R1A, PPP6C, PTPDC1, PTPN11, RAC1, RAFT, REL, RET, RHOA, RHOB, ROS1, RQCD1, RRAS2, RXRA, SF1, SF3B1, SMAD4, SMCIA, SMO, SOS1, SPOP, SS18, TAF1, TCLIA, TET2, TFG, TLX1, TPR, U2AF1, USP6, WHSC1, XPO1, ZCCHC12, ZNF133, and any combinations thereof.

In some aspects, the method further comprises amplifying the barcoded RNA after introduction to the population of cells, wherein the amplification comprises a primer specific to the first shield sequence.

In some aspects, the population of cells are primary T cells.

In some aspects, provided herein is a method of transcriptional profiling, the method comprising a) introducing a barcoded RNA library to a population of cells, wherein the barcoded RNA construct comprises (i) a first shield sequence at the 5′ end of the barcoded RNA, (ii) a unique barcode sequence, (iii) a scaffold sequence, (iv) a capture sequence, and (v) a second shield sequence at the 3′ end of the barcoded RNA; b) performing single-cell RNA sequencing on the population of cells; and c) lineage-tracing and transcriptional profiling the individual cell of the population of cells. In some aspects, the cell can be identified by the unique barcode sequence. In some aspects, an individual cell has a gene expression profile.

In some aspects, the individual cell comprises a unique genotype compared to a genotype of the population of cells.

In some aspects, over 90% of the individual cells in the population of cells are transcriptionally profiled.

In some aspects, the barcoded RNA or the barcoded RNA constructs used in the methods disclosed herein comprise any of the barcoded RNAs disclosed herein (e.g., the barcoded RNAs disclosed in section (II) above).

### IV. Polynucleotides Comprising the Barcoded RNA

In some aspects, provided herein is a polynucleotide comprising a promoter operably linked to a nucleic acid. In some aspects, the nucleic acid encodes a barcoded RNA sequence comprising a first shield sequence at the 5′ end of the barcoded RNA, a barcode sequence, a scaffold sequence, a capture sequence, and a second shield sequence at the 3′ end of the barcoded RNA.

In some aspects, the polynucleotide is a plasmid. In some aspects, the polynucleotide further comprises at least one restriction enzyme recognition sequence. In some aspects, the nucleic acid is positioned between two inverted terminal repeats (ITRs). In some aspects, the restriction enzyme recognition sequence is recognized by one or more restriction enzymes.

In some aspects, the promoter is a constitutively active promoter, a cell-type specific promoter, or an inducible promoter. In some aspects, the promoter is a Pol III promoter. In some aspects, the Pol III promoter is a U6 promoter. In some aspects, the Pol III promoter is a H1 promoter.

In some aspects, the polynucleotide comprises a barcoded RNA comprising a shield sequence (e.g., a first and/or second shield sequence). In some aspects, the shield sequence(s) protect the barcoded RNA from an endonuclease. In some aspects, the first shield sequence comprises at least one stem loop. In some aspects, the first shield sequence is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 10. In some aspects, the first shield sequence is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 10. In some aspects, the first shield sequence is between about 60% to about 100%, between about 70% to about 100%, between about 80% to about 100%, or between about 90% to about 100% identical to the nucleotide sequence set forth as SEQ ID NO: 10. In some aspects, the first shield sequence comprises the nucleotide sequence set forth as SEQ ID NO: 10. In some aspects, the first shield sequence comprises a γ-methyl phosphate cap. In some aspects, the first shield sequence is encoded by the nucleotide sequence set forth as SEQ ID NO: 1.

In some aspects, the first shield sequence is the first 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, or 50 nucleotides of the U6 non-coding small nuclear RNA.

In some aspects, the first shield sequence is at least the first 15 nucleotides, at least the first 16 nucleotides, at least the first 17 nucleotides, at least the first 18 nucleotides, at least the first 19 nucleotides, at least the first 20 nucleotides, at least the first 21 nucleotides, at least the first 22 nucleotides, at least the first 23 nucleotides, at least the first 24 nucleotides, at least the first 25 nucleotides, at least the first 26 nucleotides, at least the first 27 nucleotides, at least the first 28 nucleotides, at least the first 29 nucleotides, at least the first 30 nucleotides, at least the first 31 nucleotides, at least the first 32 nucleotides, at least the first 33 nucleotides, at least the first 34 nucleotides, at least the first 35 nucleotides, at least the first 36 nucleotides, at least the first 37 nucleotides, at least the first 38 nucleotides, at least the first 39 nucleotides, at least the first 40 nucleotides, at least the first 41 nucleotides, at least the first 42 nucleotides, at least the first 43 nucleotides, at least the first 44 nucleotides, at least the first 45 nucleotides, at least the first 46 nucleotides, at least the first 47 nucleotides, at least the first 48 nucleotides, at least the first 49 nucleotides, or at least the first 50 nucleotides of the U6 non-coding small nuclear RNA.

In some aspects, the first shield sequence is between the first 15 nucleotides to the first 50 nucleotides, between the first 15 nucleotides to the first 40 nucleotides, between the first 15 nucleotides to the first 30 nucleotides, between the first 15 nucleotides to the first 20 nucleotides, between the first 27 nucleotides to the first 50 nucleotides, between the first 27 nucleotides to the first 40 nucleotides, or between the first 20 nucleotides to the first 27 nucleotides of the U6 non-coding small nuclear RNA.

In some aspects, the second shield sequence comprises at least one stem loop. In some aspects, the second shield sequence is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 11. In some aspects, the first shield sequence is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 11. In some aspects, the first shield sequence is between about 60% to about 100%, between about 70% to about 100%, between about 80% to about 100%, or between about 90% to about 100% identical to the nucleotide sequence set forth as SEQ ID NO: 11. In some aspects, the second shield sequence comprises the nucleotide sequence set forth as SEQ ID NO: 11. In some aspects, the second shield sequence is encoded by the nucleotide sequence set forth as SEQ ID NO: 2.

In some aspects, the second shield sequence comprises an artificial sequence. In some aspects, the artificial sequence forms an artificial stem loop in the barcoded RNA. In some aspects the artificial stem loop protects the barcoded RNA from exonuclease degradation. In some aspects, the artificial stem loop protects the barcoded RNA from 3′ to 5′ exonuclease degradation.

In some aspects, the polynucleotide comprises a barcoded RNA comprising a terminator sequence. In some aspects, the second shield sequence comprises an artificial stem loop. In some aspects, the second shield sequence comprises both an artificial stem loop and a terminator sequence. In some aspects, the second shield sequence comprises an artificial stem loop at the 5′ end of the second shield sequence and a terminator sequence at the 3′ end of the second shield sequence. In some aspects, the second shield sequence comprises an artificial stem loop at the 5′ end of the second shield sequence and a terminator sequence immediately following the second shield sequence.

In some aspects, the second shield sequence is at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, or at least 50 nucleotides long.

In some aspects, the second shield sequence is between 15 nucleotides to 50 nucleotides, between 15 nucleotides to 40 nucleotides, between 15 nucleotides to 30 nucleotides, between 15 nucleotides to 20 nucleotides, between 29 nucleotides to 50 nucleotides, between 29 nucleotides to 40 nucleotides, or between 20 nucleotides to 29 nucleotides long.

In some aspects, the barcode sequence is 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, or 50 nucleotides long. In some aspects, the barcode sequence is at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, or at least 50 nucleotides long.

In some aspects, the barcode sequence is between 2 nucleotides to 50 nucleotides, between 5 nucleotides to 50 nucleotides, between 8 nucleotides to 50 nucleotides, between 10 nucleotides to 50 nucleotides, between 15 nucleotides to 50 nucleotides, between 20 nucleotides to 50 nucleotides, between 25 nucleotides to 50 nucleotides, between 30 nucleotides to 50 nucleotides, between 35 nucleotides to 50 nucleotides, between 40 nucleotides to 50 nucleotides, between 45 nucleotides to 50 nucleotides, between 5 nucleotides to 45 nucleotides, between 5 nucleotides to 40 nucleotides, between 5 nucleotides to 35 nucleotides, between 5 nucleotides to 30 nucleotides, between 5 nucleotides to 25 nucleotides, between 5 nucleotides to 20 nucleotides, between 5 nucleotides to 15 nucleotides, between 5 nucleotides to 8 nucleotides, between 8 nucleotides to 45 nucleotides, between 8 nucleotides to 40 nucleotides, between 8 nucleotides to 35 nucleotides, between 8 nucleotides to 30 nucleotides, between 8 nucleotides to 25 nucleotides, between 8 nucleotides to 20 nucleotides, between 8 nucleotides to 15 nucleotides, or between 8 nucleotides to 10 nucleotides long.

In some aspects the polynucleotide comprises a barcoded RNA comprising a scaffold sequence. In some aspects, the scaffold sequence is a single guide RNA (sgRNA). In some aspects, the sgRNA has been modified to avoid premature termination of Pol-III transcription. In some aspects, the sgRNA has been modified to delete a TTTT stretch within the stem-loop. In some aspects, the sgRNA comprises a protospacer. In some aspects the protospacer is the barcode sequence. In some aspects, the sgRNA comprises the nucleotide sequence set forth as SEQ ID NO: 13. In some aspects, the sgRNA is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 13. In some aspects, the sgRNA is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 13. In some aspects, the sgRNA is between about 60% to about 100%, between about 70% to about 100%, between about 80% to about 100%, or between about 90% to about 100% identical to the nucleotide sequence set forth as SEQ ID NO: 13. In some aspects, the sgRNA is encoded the nucleotide sequence set forth as SEQ ID NO: 4.

In some aspects, the scaffold sequence comprises a three-way junction motif. In some aspects, the scaffold sequence comprises a four-way junction motif. In some aspects, the scaffold sequence comprises a five-way junction motif.

In some aspects, the scaffold sequence is a bacteriophage pRNA. In some aspects, the bacteriophage pRNA is phi29 (F29). In some aspects, the F29 pRNA contributes to high thermodynamic stability, highly efficient complex assembly, and/or resistance to denaturation. In some aspects, the F29 pRNA comprises a first arm and a second arm. In some aspects, an aptamer has been inserted into the first arm of the F29 pRNA. In some aspects, an aptamer has been inserted into the second arm of the F29 pRNA. In some aspects, an aptamer has been inserted into both the first arm and the second arm of the F29 pRNA. In some aspects, the F29 pRNA comprises the nucleotide sequence set forth as SEQ ID NO: 14. In some aspects, the F29 pRNA is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 14. In some aspects, the F29 pRNA is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 14. In some aspects, the F29 pRNA is between about 60% to about 100%, between about 70% to about 100%, between about 80% to about 100%, or between about 90% to about 100% identical to the nucleotide sequence set forth as SEQ ID NO: 14. In some aspects, the F29 pRNA is encoded by the nucleotide sequence set forth as SEQ ID NO: 5.

In some aspects, the bacteriophage pRNA is phi30 (F30). In some aspects, the F30 pRNA comprises a first arm and a second arm. In some aspects, an aptamer has been inserted into the first arm of the F30 pRNA. In some aspects, an aptamer has been inserted into the second arm of the F30 pRNA. In some aspects, an aptamer has been inserted into both the first arm and the second arm of the F30 pRNA. In some aspects, the F30 pRNA comprises the nucleotide sequence set forth as SEQ ID NO: 15. In some aspects, the F30 pRNA is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 15. In some aspects, the F30 pRNA is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 15. In some aspects, the F30 pRNA is between about 60% to about 100%, between about 70% to about 100%, between about 80% to about 100%, or between about 90% to about 100% identical to the nucleotide sequence set forth as SEQ ID NO: 15. In some aspects, the F30 pRNA is encoded by the nucleotide sequence set forth as SEQ ID NO: 6.

In some aspects, the capture sequence comprises the nucleotide sequence set forth as SEQ ID NO: 12. In some aspects, the capture sequence is at least 60%, at least 65%, at least 70%, at least 75%, at least 80%, at least 85%, at least 90%, at least 95%, at least 96%, at least 97%, at least 98%, at least 99%, or 100% identical to the nucleotide sequence set forth as SEQ ID NO: 12. In some aspects, the capture sequence is about 60%, about 65%, about 70%, about 75%, about 80%, about 85%, about 90%, about 95%, about 96%, about 97%, about 98%, about 99%, or 100% identical to the nucleotide sequence set forth as SEQ ID NO: 12. In some aspects, the capture sequence is between about 60% to about 100%, between about 70% to about 100%, between about 80% to about 100%, or between about 90% to about 100% identical to the nucleotide sequence set forth as SEQ ID NO: 12. In some aspects, the capture sequence is encoded by the nucleotide sequence set forth as SEQ ID NO: 3.

In some aspects, the capture sequence can be recognized by an entity. In some aspects, the recognition allows the barcoded RNA to be separated from non-barcoded RNA. In some aspects, the entity is a nucleic acid. In some aspects, the nucleic acid is a primer. In some aspects, the primer allows for reverse transcription of the construct. In some aspects, the nucleic acid is an oligonucleotide that hybridizes to the capture sequence. In some aspects, the oligonucleotide comprises a label. In some aspects, the label is a radioactive phosphate, biotin, a fluorophore, chemical tag, antibody, or an enzyme. In some aspects, the label is biotin. In some aspects, the biotin may be captured by streptavidin. In some aspects, the streptavidin is conjugated to a bead.

In some aspects, the polynucleotide comprises a barcoded RNA further comprising a RNA aptamer. In some aspects, the RNA aptamer is a fluorescent RNA aptamer. In some aspects, the fluorescent RNA aptamer is a Broccoli RNA aptamer, a Spinach RNA aptamer, a Pepper RNA aptamer, a Mango II RNA aptamer, a malachite green aptamer. In some aspects, the fluorescent RNA aptamer is a Broccoli RNA aptamer. In some aspects, the Broccoli RNA aptamer sequence has been optimized.

In some aspects, the barcoded RNA is administered in the absence of a Cas protein (e.g., a Cas9). In some aspects, the barcoded RNA is stable in the absence of a Cas9 protein.

### V. Libraries and Cells Expressing Barcoded RNA

Certain aspects of the disclosure are directed to libraries and cells expressing the barcoded RNA. The libraries may comprise any of the barcoded RNAs described herein.

In some aspects, provided herein are cells expressing a barcoded RNA. In some aspects, the cell expressing a barcoded RNA comprises a first shield sequence at the 5′ end of the barcoded RNA, a barcode sequence, a scaffold sequence, a capture sequence, and a second shield sequence at the 3′ end of the barcoded RNA.

In some aspects, provided herein is a library comprising a plurality of barcoded RNA. In some aspects, the library comprises a plurality of barcoded RNAs in which the barcoded RNA comprises a first shield sequence at the 5′ end of the barcoded RNA, a barcode sequence, a scaffold sequence, a capture sequence, and a second shield sequence at the 3′ end of the barcoded RNA.

In some aspects, the library comprises a barcoded RNA comprising a sgRNA scaffold sequence. In some aspects, the library comprises a barcoded RNA comprising a F29 scaffold sequence. In some aspects, the library comprises a barcoded RNA comprising a F30 scaffold sequence. In some aspects, the library comprises barcoded RNAs comprising a sgRNA scaffold sequence, a F29 scaffold sequence, a F30 scaffold sequence, or any combination thereof.

In some aspects, library is prepared using a 5′ shield-specific primer. In some aspects, the 5′ shield-specific primer increases the specificity of the barcoded RNA amplification during sequencing library generation. In some aspects, the increased specificity results in a higher level of sequencing saturation and a higher level of recovered barcodes.

In some aspects, the library comprises at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, or at least 10000 unique barcoded RNAs. In some aspects, the library comprises about 100, about 200, about 300, about 400, about 500, about 600, about 700, about 800, about 900, about 1000, about 2000, about 3000, about 4000, about 5000, about 6000, about 7000, about 8000, about 9000, or about 10000 unique barcoded RNAs. In some aspects, the library comprises between about 100 to about 10000, between about 100 to about 9000, between about 100 to about 8000, between about 100 to about 7000, between about 100 to about 6000, between about 100 to about 5000, between about 100 to about 4000, between about 100 to about 3000, between about 100 to about 2000, between about 100 to about 1000, between about 500 to about 10000, between about 1000 to about 10000, between about 2000 to about 10000, between about 3000 to about 10000, between about 4000 to about 10000, between about 5000 to about 10000, between about 6000 to about 10000, between about 7000 to about 10000, between about 8000 to about 10000, or between about 9000 to about 10000 unique barcoded RNAs.

In some aspects, the library is a viral library. In some aspects, the viral library is a lentiviral library. In some aspects, the library is a single-cell RNA sequencing library. The single-cell RNA sequencing library may be generated by 3′ digital gene expression (DGE), SMART-seq2, SeqWell, droplet microfluidic barcoding, split and pool barcoding, or combinatorial indexing. In certain embodiments, the single-cell RNA sequencing library is an ATAC sequencing library.

In some aspects, the viral library comprises at least a first barcoded RNA and a second barcoded RNA. In some aspects, the first barcoded RNA and the second barcoded RNA are the same. In some aspects, the first barcoded RNA and the second barcoded RNA are different. In some aspects, the first barcoded RNA comprises a sgRNA scaffold. In some aspects, the first barcoded RNA comprises a F29 scaffold. In some aspects, the first barcoded RNA comprises a F30 scaffold. In some aspects, the second barcoded RNA comprises a sgRNA scaffold. In some aspects, the second barcoded RNA comprises a F29 scaffold. In some aspects, the second barcoded RNA comprises a F30 scaffold. In some aspects, the first barcoded RNA comprises a F30 scaffold and the second barcoded RNA comprises a sgRNA scaffold. In some aspects, the first barcoded RNA comprises a F29 scaffold and the second barcoded RNA comprises a sgRNA scaffold. In some aspects, the first barcoded RNA comprises a F30 scaffold and the second barcoded RNA comprises a F29 scaffold.

In some aspects, provided herein are methods of multiplexing samples for single cell sequencing. In some aspects, the method of multiplexing samples for single cell sequencing comprises labeling single cells from a plurality of samples with a barcoded RNA, and constructing a multiplexed single cell sequencing library for the plurality of samples comprising the cell of origin barcodes. In some aspects, the barcoded RNA comprises a 5′ shield sequence, a barcode sequence, a scaffold sequence, a capture sequence, and a 3′ shield sequence. In some aspects, the barcode sequence comprises a unique barcode sequence and a cell of origin barcode sequence.

In some aspects, the method further comprises sequencing the library and demultiplexing in silico based on the cell of origin barcodes and the unique barcode sequence. In some aspects, the single cell sequencing is performed with a single-cell sequencing platform (e.g., 10× Genomics® Chromium®).

In some aspects, the sample is a single nuclei or membrane bound organelle. In some aspects, the single nuclei or membrane bound organelles are labeled with a sample barcode oligonucleotide.

### VI. Kits Comprising Barcoded RNA

Certain aspects of the disclosure are directed to kits comprising the barcoded RNA. The libraries may comprise any of the barcoded RNAs or polynucleotides described herein. In some aspects, provided herein are kits comprising expression constructs. In some aspects, the expression construct comprises a promoter operably linked to a nucleic acid encoding a barcoded RNA sequence. In some aspects, the barcoded RNA sequences comprises a first shield sequence at the 5′ end of the barcoded RNA, a barcode sequence, a scaffold sequence, a capture sequence, and a second shield sequence at the 3′ end of the barcoded RNA.

In some aspects, the expression construct is a plasmid. In some aspects, the kit further comprises a viral vector. In some aspects, the viral vector is an adeno-associated viral (AAV) vector, an adenoviral vector, a lentiviral vector, or a retroviral vector. In some aspects, the viral vector comprises the expression construct.

In some aspects, the expression construct further comprises at least one restriction enzyme recognition sequence. In some aspects, the restriction enzyme recognition sequence is recognized by one or more restriction enzymes.

In some aspects, the kit comprises a library. In some aspects, the library comprises a plurality of barcoded RNAs. In some aspects, the barcoded RNA comprises a first shield sequence at the 5′ end of the barcoded RNA, a barcode sequence, a scaffold sequence, a capture sequence, and a second shield sequence at the 3′ end of the barcoded RNA.

In some aspects, the library in the kit comprises at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, or at least 10000 unique barcoded RNAs. In some aspects, the library in the kit comprises about 100, about 200, about 300, about 400, about 500, about 600, about 700, about 800, about 900, about 1000, about 2000, about 3000, about 4000, about 5000, about 6000, about 7000, about 8000, about 9000, or about 10000 unique barcoded RNAs. In some aspects, the library in the kit comprises between about 100 to about 10000, between about 100 to about 9000, between about 100 to about 8000, between about 100 to about 7000, between about 100 to about 6000, between about 100 to about 5000, between about 100 to about 4000, between about 100 to about 3000, between about 100 to about 2000, between about 100 to about 1000, between about 500 to about 10000, between about 1000 to about 10000, between about 2000 to about 10000, between about 3000 to about 10000, between about 4000 to about 10000, between about 5000 to about 10000, between about 6000 to about 10000, between about 7000 to about 10000, between about 8000 to about 10000, or between about 9000 to about 10000 unique barcoded RNAs.

In some aspects, the library in the kit is a viral library. In some aspects, the viral library is a lentiviral library. In some aspects, the library is a single-cell RNA sequencing library.

The following examples are illustrative and do not limit the scope of the claimed aspects.

### EXAMPLES

### Example 1a. Unmodified Single Guide RNAs (sgRNAs) as Genetic Barcodes

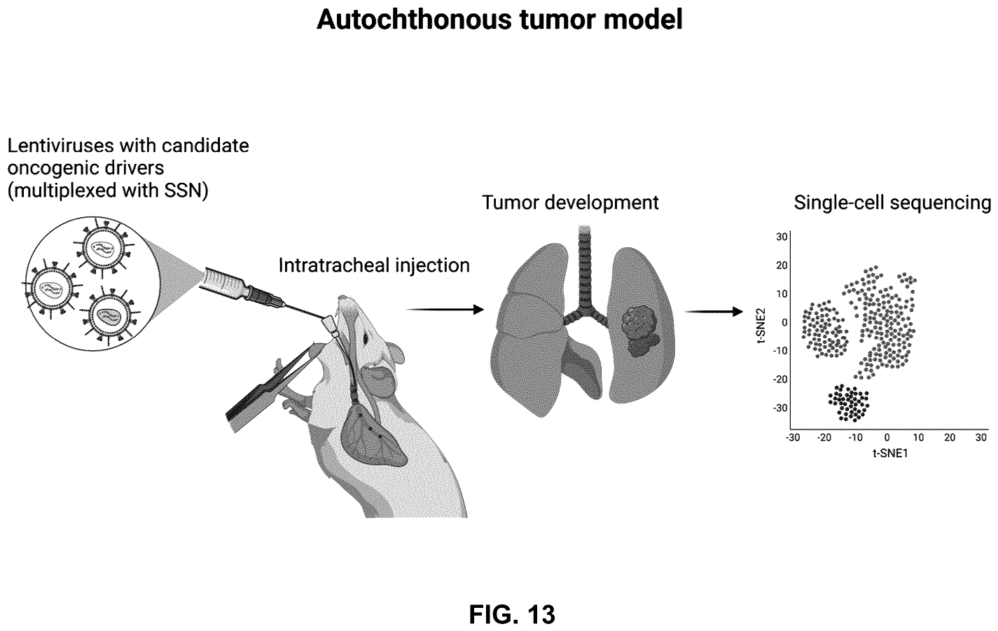

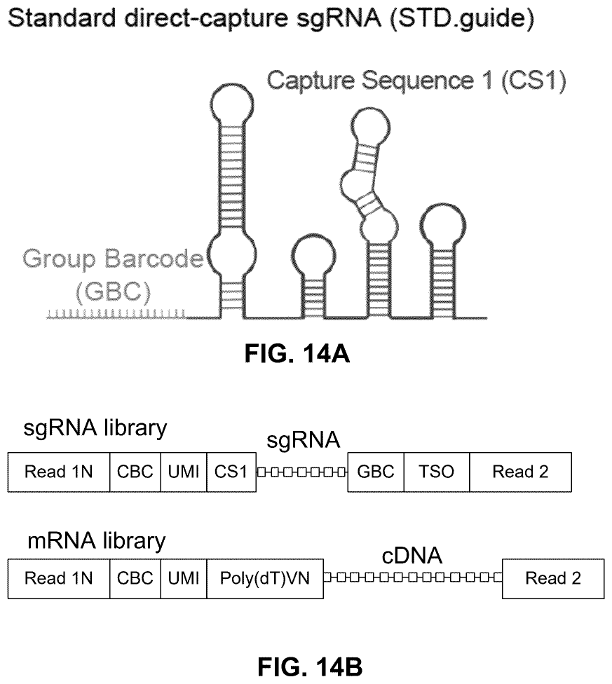

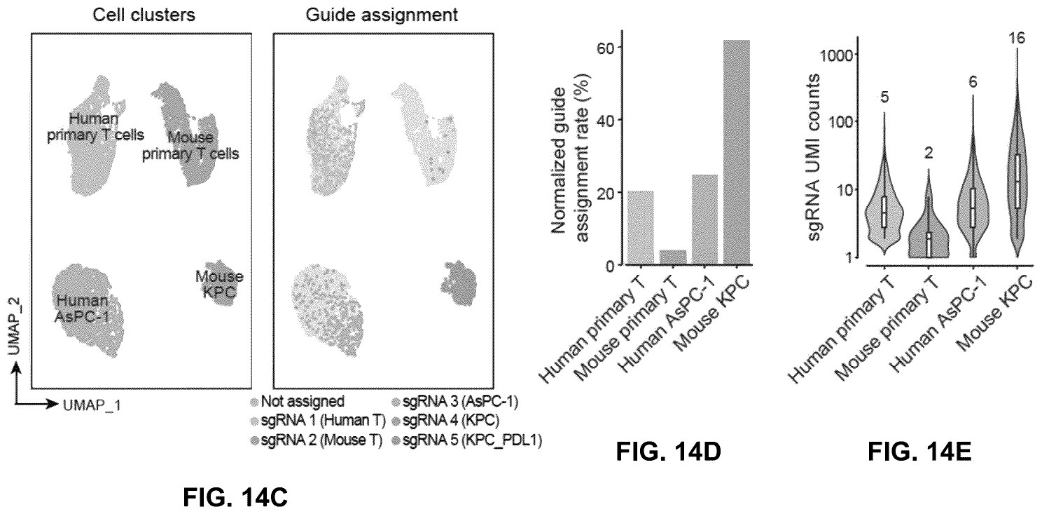

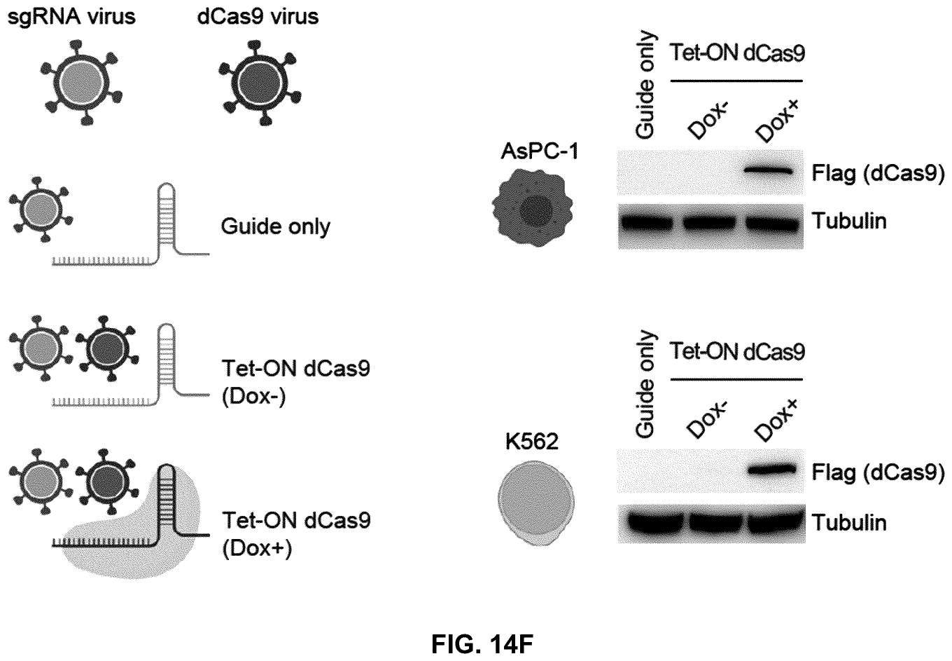

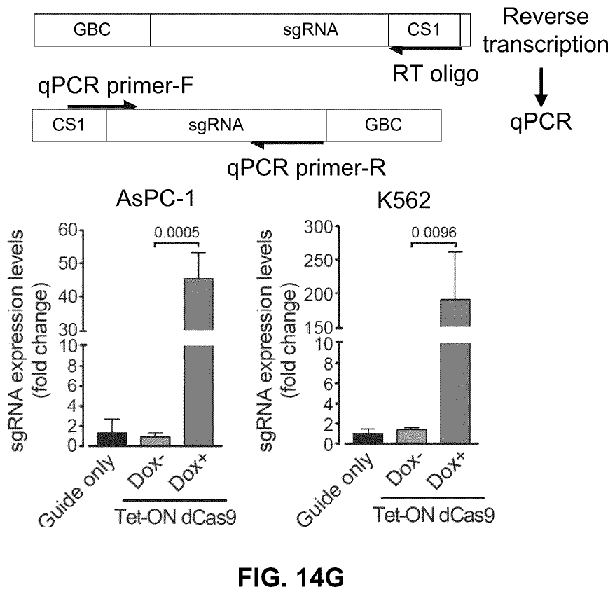

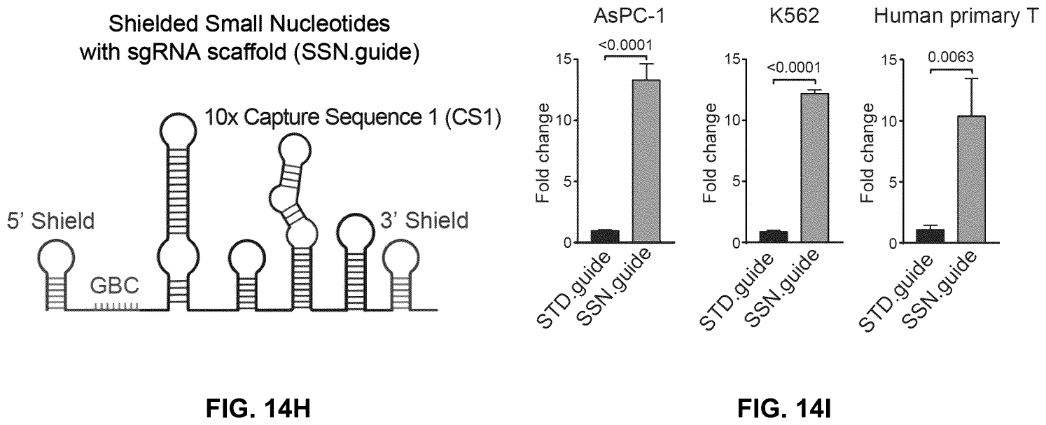

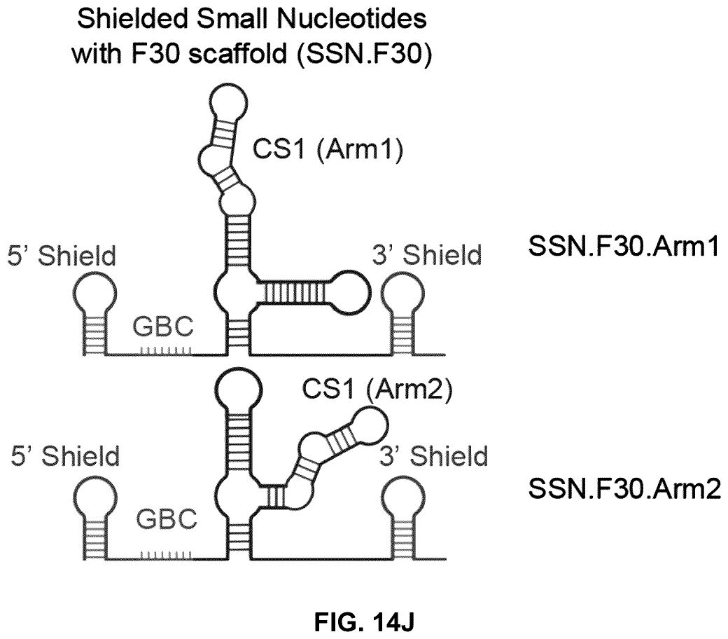

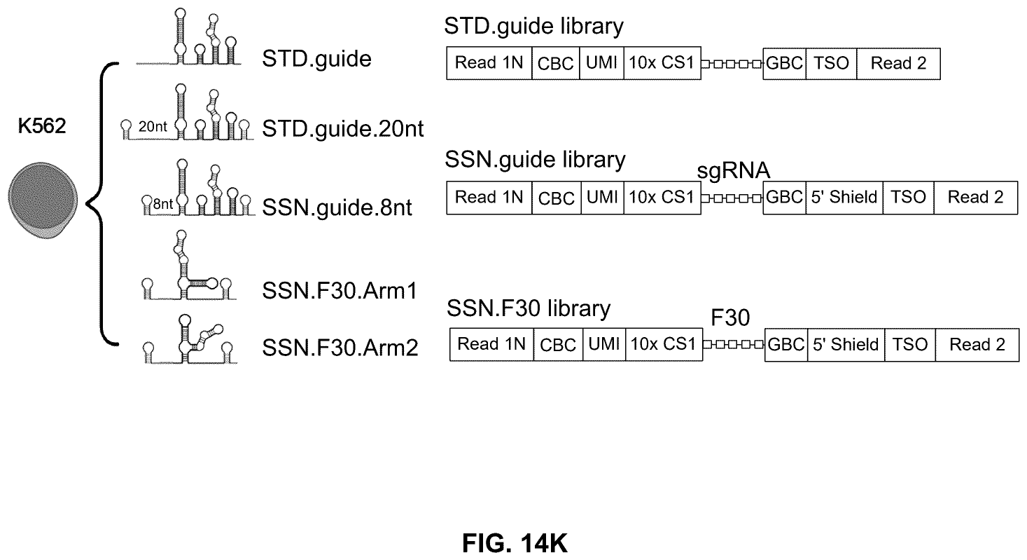

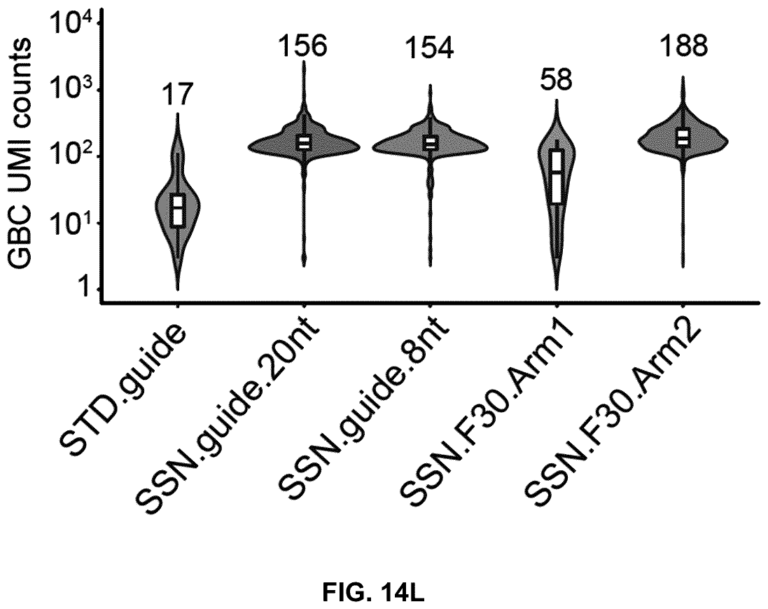

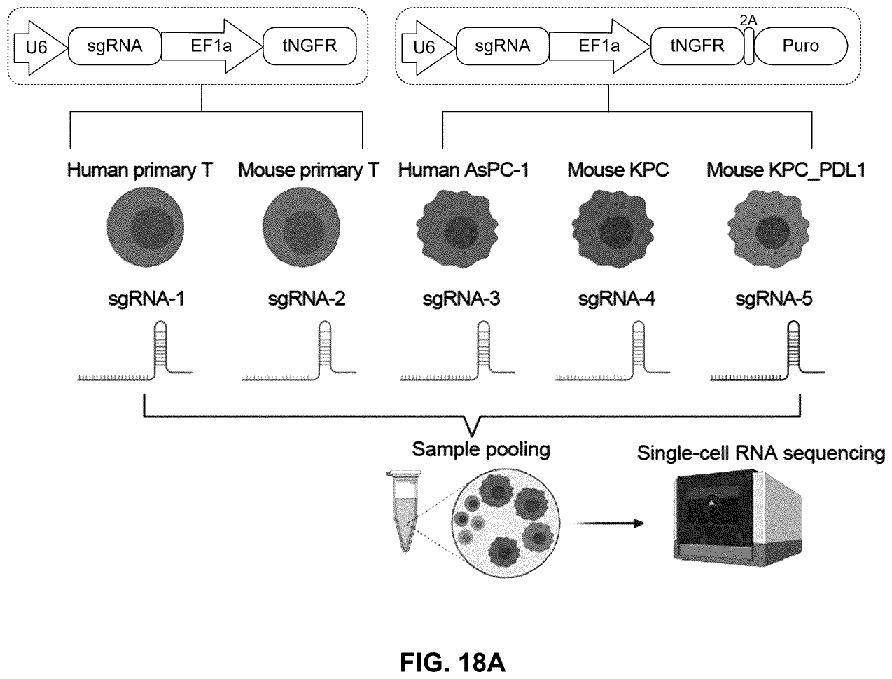

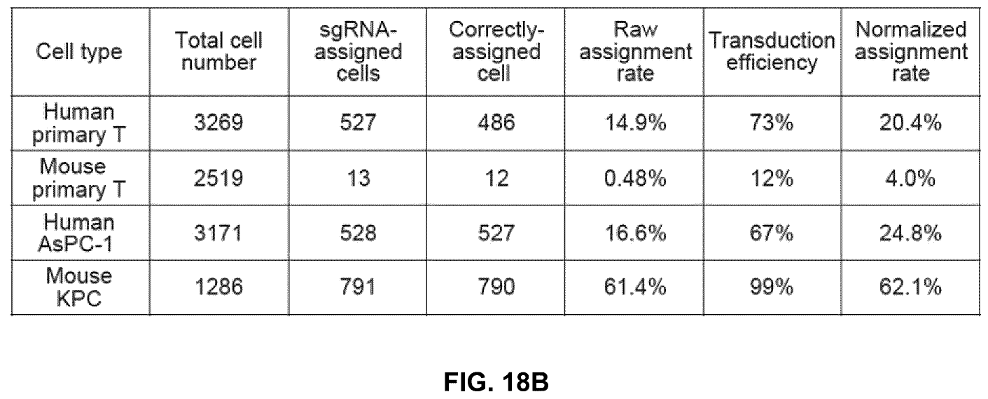

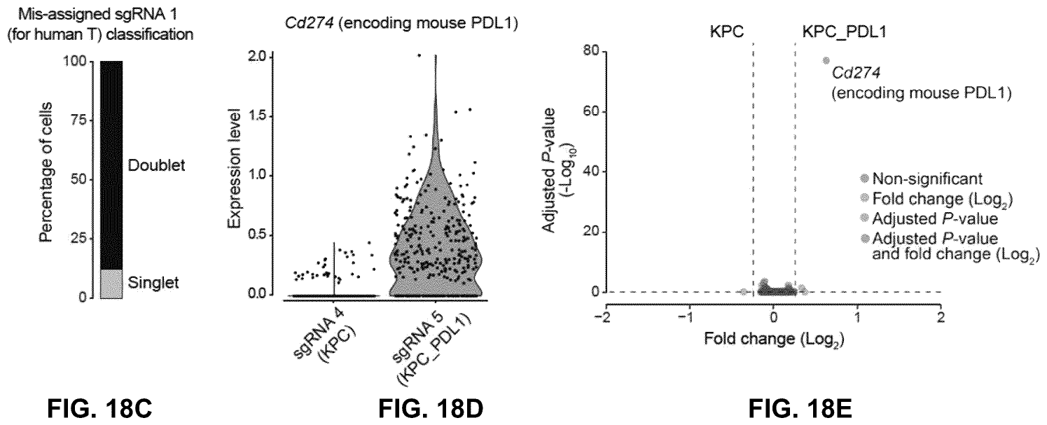

To investigate the use of unmodified single guide RNAs (sgRNAs) as genetic barcodes, non-targeting sgRNAs were designed following a standard design compatible with the direct-capture Perturb-seq (FIG. 14A). Unique sgRNAs were transduced into four different types of cells: human primary T cells, mouse primary T cells, human pancreatic cancer line AsPC-1 and mouse pancreatic cancer KPC cells (FIG. 18A). To examine if the sgRNA barcoding was able to efficiently identify homotypic cells, mouse KPC cells were further divided into two groups: control (KPC_WT) and ectopically expressing mouse PDL1 (KPC_PDL1), each with a unique sgRNA. All cells were pooled and subjected to single cell RNA sequencing (scRNA-seq) following 10× Genomics single cell 3′ gene expression with feature barcoding technology workflow (schematics of sgRNA and mRNA libraries shown in FIG. 13B).

The results indicated that each sgRNA was assigned correctly to their corresponding cell types (FIG. 14C), with a minimal number of misassigned cells that mainly were classified as doublets (FIG. 18B-18C). For homotypic cells, the sgRNA tags separated KPC and KPC_PDL1 cells, as confirmed by differential expression of Cd274 (encoding mouse PDL1) (FIG. 18D-18E).

Next, to compare the sgRNA assignment rate in different cells, the results were normalized to account for varying lentiviral transduction efficiency particularly for mouse primary T cells (FIG. 18B). The analysis revealed that despite the accuracy, the overall recovery of the sgRNA-assigned cells was inadequate ranging between 3-20% for mouse and human primary T cells and 20-60% for cancer cells (FIG. 14D). The low sgRNA assignment rate dampened the demultiplexing capacity as the majority of cells failed to be associated with a group barcode corresponding with lineage identities (FIG. 18B).

It was reasoned that the inept sgRNA recovery was due to expression of sgRNAs below the detection threshold in a large proportion of successfully barcoded cells. This was supported by the results that showed the distribution pattern of sgRNA unique molecular identifiers (UMIs) detected in each cell type mirrored the recovery rate (FIG. 14E). Considering that previous direct sgRNA captures were performed exclusively in the presence of Cas9, one possible explanation for the low recovery was reduced sgRNA stability in the absence of Cas9 as previously noted in U2OS cells. To corroborate those observations, a doxycycline-inducible expression of catalytically dead Cas9 (dCas9) was introduced to sgRNA-expressing AsPC-1 and K562, a chronic myelogenous leukemia (CML) cell line frequently used for direct sgRNA capture experiments in scRNA-seq (FIG. 14F). Quantitative expression analysis indicated that dCas9 expression led to 45-fold and 190-fold increase of sgRNA levels detected in AsPC-1 and K562, respectively (FIG. 14G). Although Cas9 protects sgRNA from degradation it is also a significant confounding factor (large size xenoprotein) limiting various applications especially in primary immune cells.

Taken together, these results indicated that in the absence of Cas9 the standard direct-capture sgRNA-barcoding technology is inadequate to efficiently record cell identities in scRNA-seq analyses. Therefore, new methods were explored to increase stability of sgRNA independent of the Cas9 protein.

### Example 1B. Intracellular Barcoding with Shielded Small Nucleotides

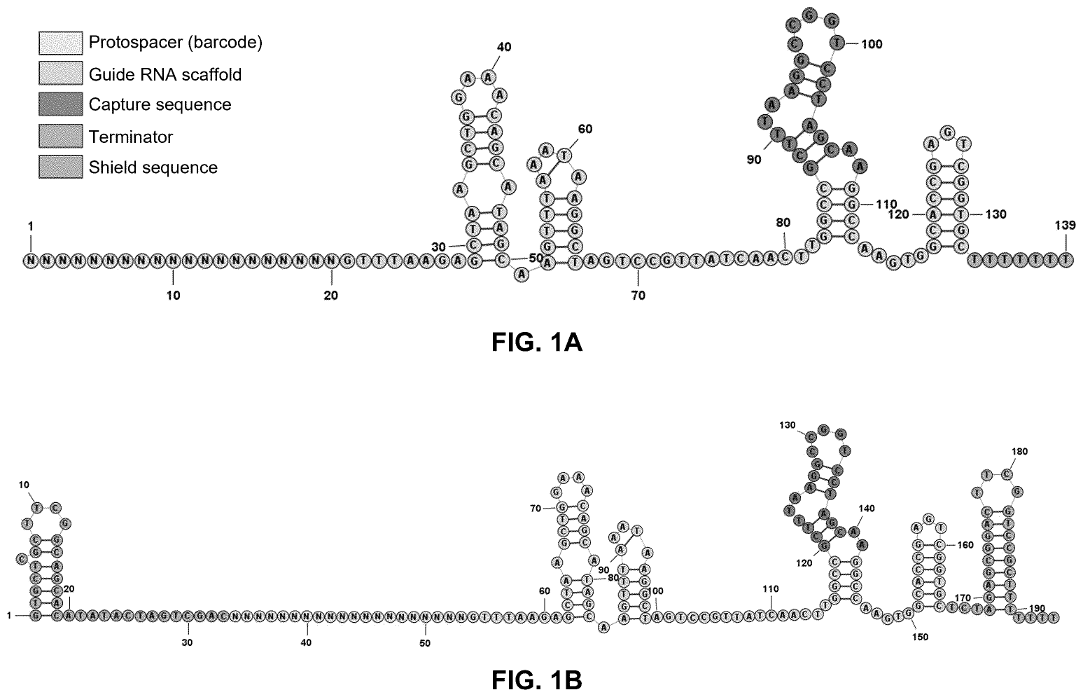

Shielded small nucleotide (SSN) barcoding transcripts were designed and constructed for intracellular barcoding. SSN barcoding transcripts composed of the first 27 nucleotides of the U6 snRNA, an 8 to 20 nucleotide sample barcode, a scaffold, a capture sequence, an artificial stem, and a terminator were prepared. The scaffold was derived from either a single-guide RNA (sgRNA) or a bacteriophage pRNA (e.g., F29, F30). FIG. 1 displays exemplary SSN barcoding transcripts. In some constructs, an artificial stem was further incorporated to the 3′ end to protect the transcripts against 3′-5′ exonuclease attack (FIG. 14H).

The SSN barcoding transcripts were generated using either lentiviral or retroviral vectors carrying a Pol III promoter (e.g., U6 promoter), or through in vitro transcription. Cells carrying SSN barcoding transcripts are compatible with commercial single-cell RNA sequencing platforms (e.g., 10× Genomics® Chromium®). More specifically, the SSN barcodes are captured directly by Chromium® Single Cell 3′ v3 Gel Beads, together with poly-adenylated mRNAs. Gene expression profiles (3′ gene expression library) were obtained simultaneously with pre-assigned sample barcodes (SSN-seq barcode library) from the individual cell, thus proving a multiplexed, high-throughput approach for single-cell RNA sequencing.

The shielded small nucleotides (SSN) derived from sgRNAs (SSN.guide) were introduced into AsPC-1, K562 and human primary T cells, to compare their stability to standard sgRNAs (STD.guide).

K562 (human chronic myelogenous leukemia) cells were grown in RPMI 1640 medium supplemented with 10% fetal bovine serum (FBS) and 100 U/mL penicillin/streptomycin. Human embryonic kidney (HEK) 293T, AsPC-1 (human pancreatic cancer), EL4 (mouse lymphoma) cells and KPC (mouse pancreatic cancer) cells were maintained in DMEM supplemented with 10% FBS and 100 U/mL penicillin/streptomycin. All cells were cultured at 37° C. in a humidified incubator with 5% CO2.

Experiment using standard sgRNAs. Human and mouse primary T, AsPC-1 and mouse KPC (KPC_WT and KPC_PDL1) cells were transduced with lentiviral vectors indicated in FIG. 18A. AsPC-1 and KPC cells were further selected in media containing 2 μg/ml puromycin. On the day of sample collection, cells were counted, and KPC_WT and KPC_PDL1 cells were mixed first at a 1:1 ratio. Human Primary T, mouse primary T, AsPC-1 and KPC cells were then pooled at approximately 2:2:2:1 ratio and subjected to scRNA-seq.

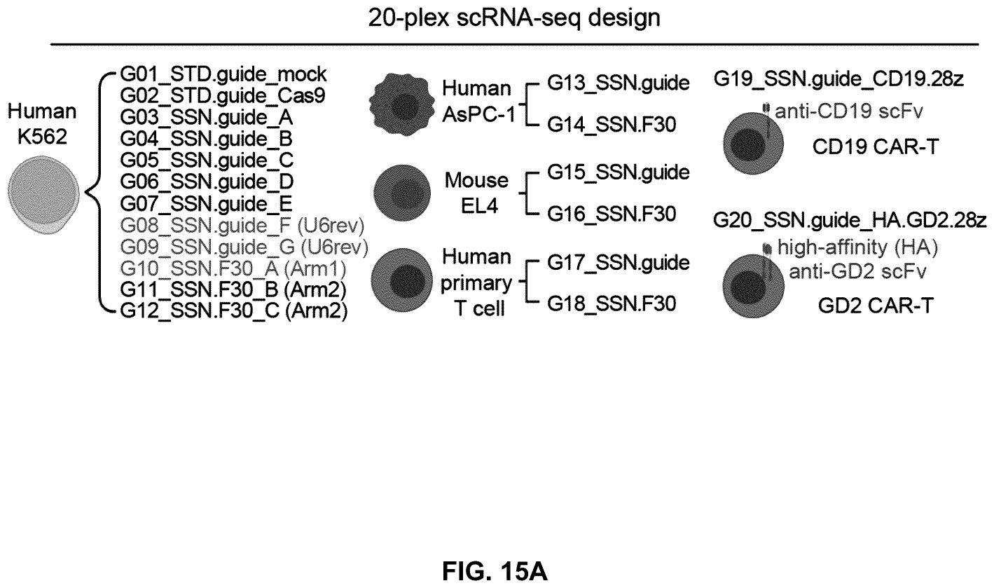

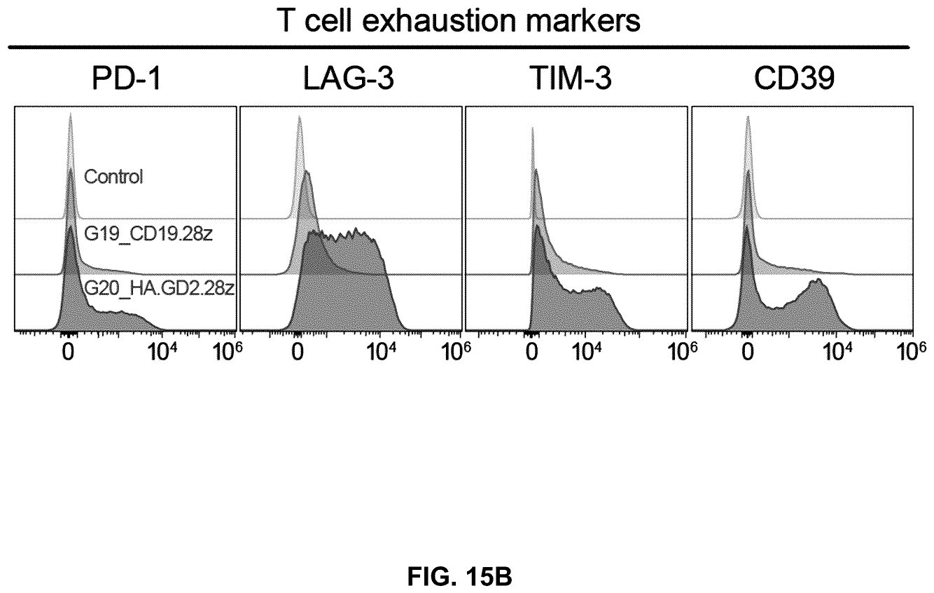

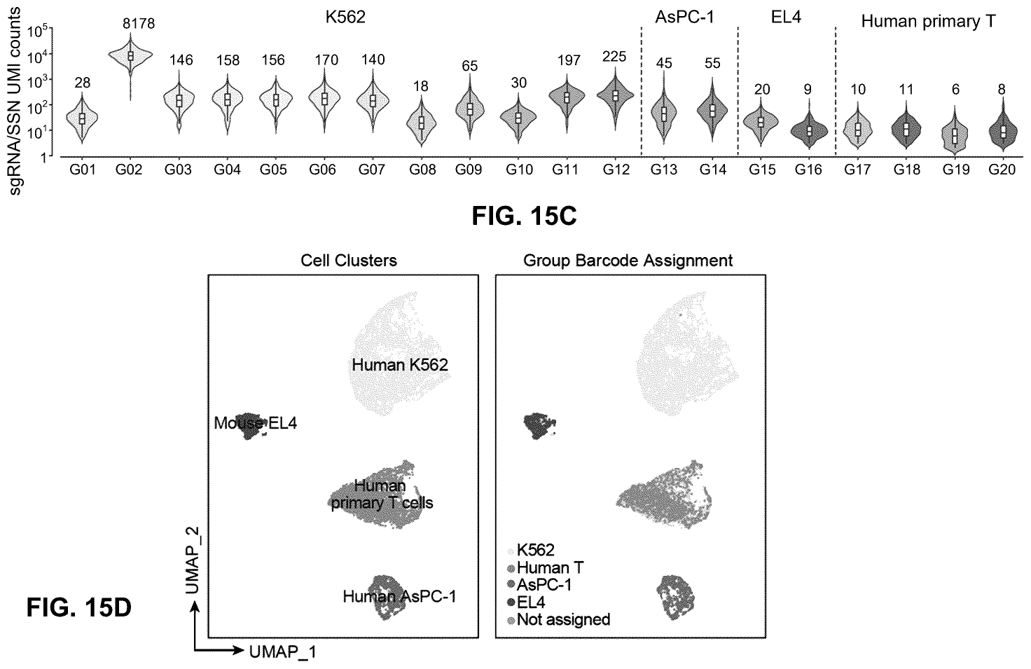

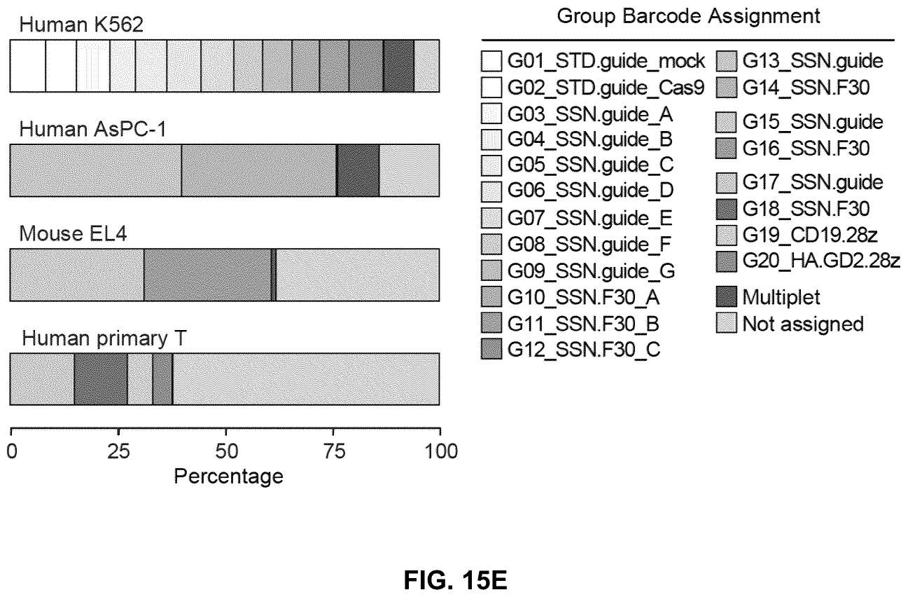

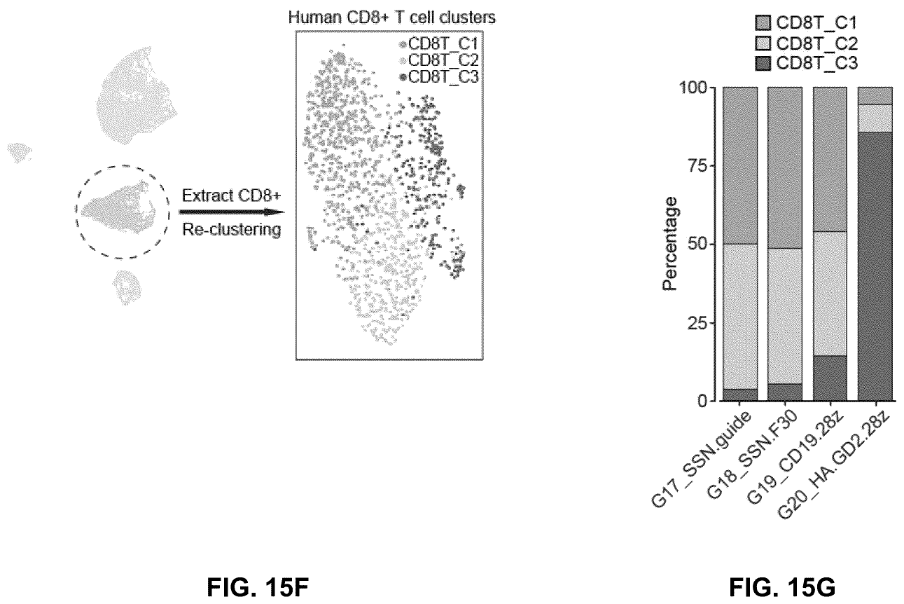

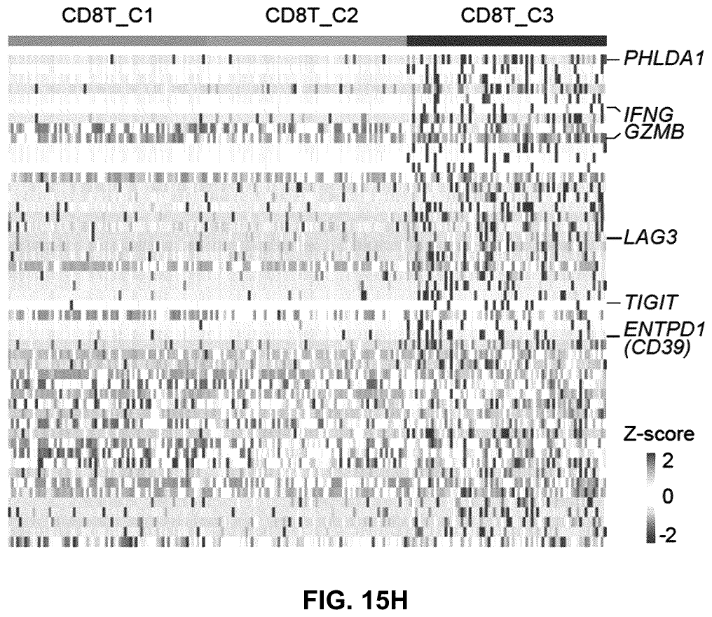

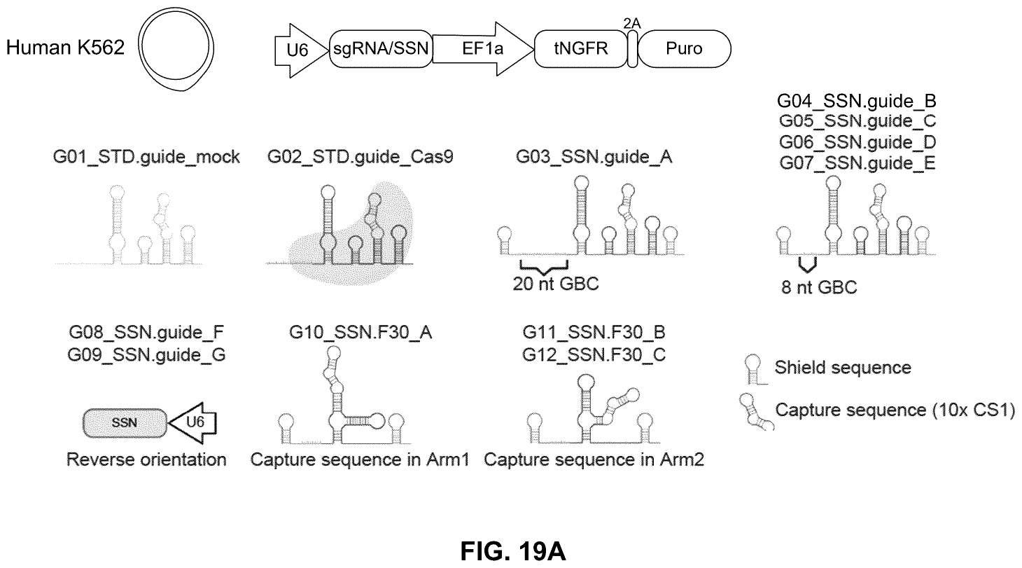

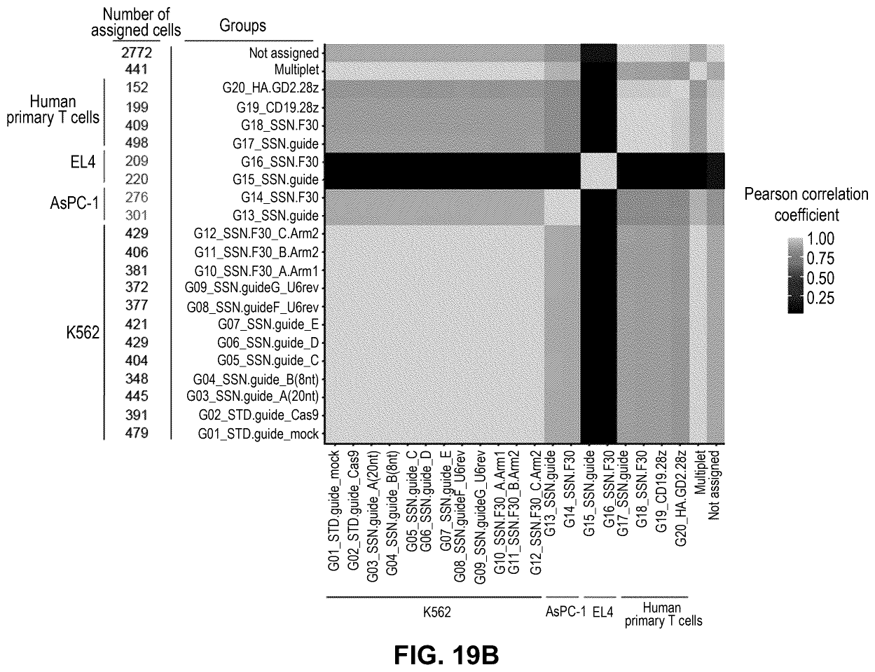

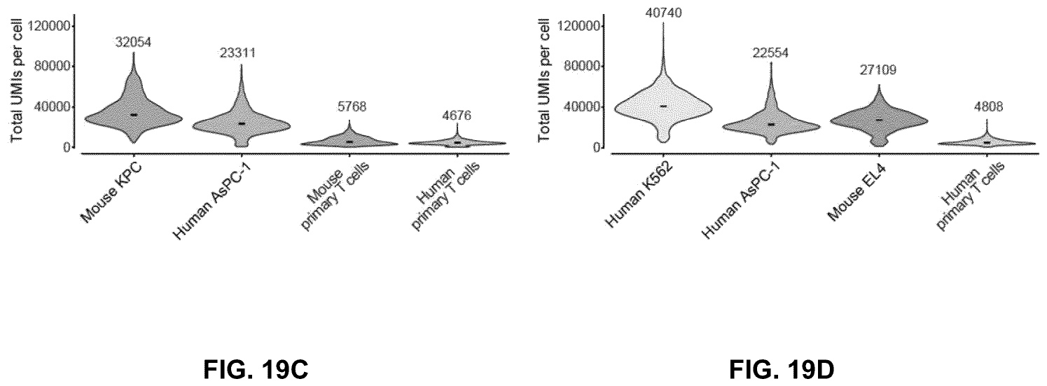

Experiment using 20-plex mixed-species/types. At day 0, K562, AsPC-1, mouse EL4 cell lines and human primary T cells were transduced with lentiviral vectors (see FIG. 15A and FIG. 19A). Puromycin selection (2 μg/ml) was applied to all groups except the four groups of human primary T cells. At day 10, expression of immune checkpoints (PD-1, LAG-3, TIM-3 and CD39) was verified in CD19.28z (G19) and HA.GD2.28z (G20) CAR T cells using flow cytometry. The two CAR T groups (G19 and G20) were then pooled with two non-CAR groups (G17 and G18) at equal ratios. The combined T cells were further FACS-sorted to enrich lentivirally transduced population expressing a cell surface reporter tNGFR. In total twelve experimental groups of K562 (G01-G12), two groups of AsPC-1 (G13-G14) and two groups of EL4 (G15-16) cells lines pooled at equal ratios (12:2:2) were analyzed. Due to anticipated lower barcode assignment rates for human primary T cells, double the amount of T cells per group (four groups in total) were used in comparison to tumor cell lines resulting in a 12:2:2:8 ratio for each cell type.

The results showed a 10- to 13-fold increase of detected gRNAs when armored with the shield sequences across different cell types (FIG. 14I), showing the SSN.guide could improve the recovery rate of barcoded cells during scRNA-seq.

Next, alternative scaffold modifications were explored based on the thermodynamically stable three-way junction (3WJ) motif of the motor pRNA of bacteriophage @, which contained two stem loops (denoted as arms) that accommodate the capture sequence insertion. A 3WJ-derived F30 sequence with enhanced scaffolding capacity and stability to generate shielded small nucleotides (SSN.F30) was utilized. The group barcode was placed immediately after the 5′ shield sequence, while the capture sequence was inserted into either Arm 1 or Arm 2 of the F30 scaffold (FIG. 14J).

The performance of a standard sgRNA (STD.guide, see FIG. 13A) was compared to the modified versions including: shielded small nucleotides sgRNA with 20-nt group barcode (SSN.guide.20nt) or with 8-nt group barcode (SSN.guide.8nt) and a shielded small nucleotides sgRNA containing F30-scaffold with the capture sequence inserted into Arm1 (SSN.F30.Arm1) or Arm2 (SSN.F30.Arm2). Direct-capture scRNA-seq was performed using K562 cells and the sgRNA/SSN libraries were sequenced (FIG. 14K).

Consistent with qPCR results, the shielded sgRNA showed nearly 10-fold increase in abundance (median of 156 UMIs) than the standard sgRNA (median of 17 UMIs). The 20-nt and 8-nt group barcodes had comparable UMI counts (median: 156 vs. 154), suggesting the length of the group barcode could be flexible. The F30-derived SSNs were also captured successfully, with a higher level in the group where the capture sequenced was inserted into the Arm2 (median of 188 UMIs) rather than Arm1 (median of 58 UMIs) (FIG. 14L). Notably, the UMI counts for SSN.F30.Arm2 were generally comparable with the ones for SSN.guide (median: 188 vs. 154). These results demonstrate that the engineered SSNs can be used as standalone genetic barcodes for scRNA-seq.

### Example 2. Lentiviral Plasmid Construction for Shielded sgRNAs and Shielded F30-Derived Small RNAs

To generate barcoded RNAs, lentiviral plasmids for sample barcoding by conventional sgRNAs (pSSN-guide), lentiCRISPR v2 (Addgene® plasmid #52961) were used as a backbone. The cassettes comprising human U6 promoter-filler-sgRNA scaffold (from lentiCRISPR v2), human EF-1alpha promoter (from pCDH-EF1, Addgene® plasmid #72266), truncated human nerve growth factor receptor (tNGFR) (from MSGV Hu Acceptor PGK-NGFR, Addgene® plasmid #64270) and P2A-Puromycin (from lentiCRISPR v2) were amplified using Phusion Green Hot Start II High-Fidelity PCR Master Mix (Thermo®) and were assembled into the backbone by Gibson Assembly (NEB). The modified sgRNA scaffold carrying the capture sequence template (GCTTTAAGGCCGGTCCTAGCAA (SEQ ID NO:3) was included on overlapping PCR primers for Gibson Assembly. Annealed oligonucleotides for different sgRNAs were ligated into BsmBI-digested pSSN-guide vectors.

For constructing shielded sgRNAs, barcodes with 8 nucleotides or 20 nucleotides were appended onto the PCR primer to amplify the sgRNA from pSSN-guide. The PCR products were then ligated into pAVU6+27-F30-2xdBroccoli (Addgene® plasmid #66842) between the SalI and XbaI restriction sites. PCR amplicons comprising U6 promoter-driven shielded sgRNAs were cloned into pSSN-guide, replacing the original U6-sgRNA cassette to generate pSSN-shield.

For F30-derived shielded small RNAs, pAV-U6+27-Tornado-F30-Broccoli-empty (Addgene® Plasmid #124361) was used as a template for the F30 scaffold. The capture sequence was introduced within two partially complementary primers to amplify the whole plasmid followed by Gibson Assembly, resulting in insertion of the capture sequence into Arm1 or Arm2 of the F30 scaffold. A similar strategy was used to insert the fluorescent RNA aptamer Broccoli. U6 promoter-driven F30-derived small RNAs were then cloned into pSSN-guide to generate pSSN-F30.

Chimeric Antigen Receptor (CAR) constructs were prepared. In particular, the sequence for CD19-specific FCM63 scFv was amplified from the plasmid pHR_PGK_antiCD19_synNotch_Gal4VP64 (Addgene Plasmid #79125) 77. Codon-optimized cDNAs encoding GD2-specific 14g2a-E101K scFv, CD28 and CD32 signaling domains were synthesized by Twist Bioscience. The cDNAs were further assembled into pXL_SSN.guide, replacing the puromycin cassette to create constructs carrying CD19.28z or HA.GD2.28z constructs for SSN-seq. For dual SSN barcoding for the 8-plex CAR T cells, constructs with CD19.28z carrying eight SSN.guide barcodes were generated first. A modified mouse U6 promoter derived from pMJ179 (Addgene plasmid #85996), along with a SSN.F30 cassette, was cloned into an intermediate vector. Eight mU6-SSN.F30 cassettes derived from corresponding intermediate constructs were then ligated separately into the vectors carrying hU6-SSN.guide and CD19.28z, to generate pXL_dSSN_CD19.28z.

HEK293T cells were transfected with lentiviral transfer and packaging plasmids using TransIT-Lenti (Mirus Bio) following the manufacturer's protocol. Lentiviral supernatants were collected 48 hours post-transfection and concentrated by centrifugation. The concentrated lentiviruses were resuspended in cell culture medium and stored at −80° C.

### Example 3. Intracellular Barcoding with Shielded sgRNA does not Require Cas9

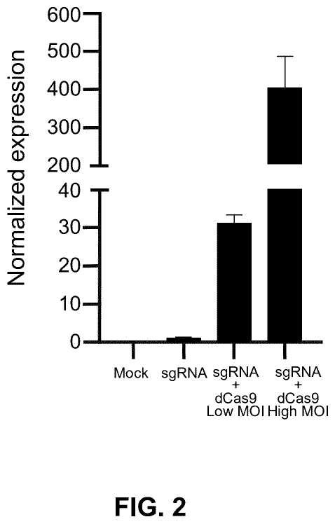

The stability of the shielded sgRNA was analyzed to determine if it was stable in the absence of Cas9. Conventional sgRNAs require Cas9 for stability. See FIG. 2 and Example 1A.

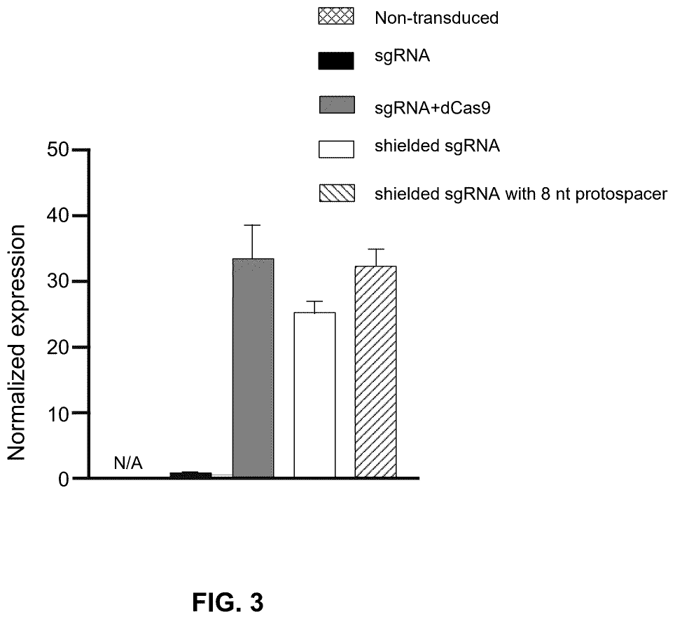

A shielded sgRNA was designed to express high-level transcripts by Pol III promoters. The first 27 nucleotides of human U6 small nuclear RNA were added to the 5′ end of the sgRNA. U6 promoter-driven small RNAs comprising the first 27 nucleotides of human U6 small nuclear RNA (snRNA) were capped with γ-methyl phosphates and accumulated to higher levels than unmodified ones. Next, an artificial stem was further incorporated to the 3′ end of the sgRNA to protect the transcripts against 3′-5′ exonuclease attack. Two variations of the construct were created in which the protospacer length on the sgRNA was decreased to 8 nucleotides or unmodified at 20 nucleotides. The resulting “shielded” sgRNAs were then transduced into K562 cells and the expression level was compared with the original sgRNA. The shielded sgRNAs had ˜25 to 30-fold higher expression levels as compared to the sgRNA alone in the absence of Cas9. See FIG. 3.

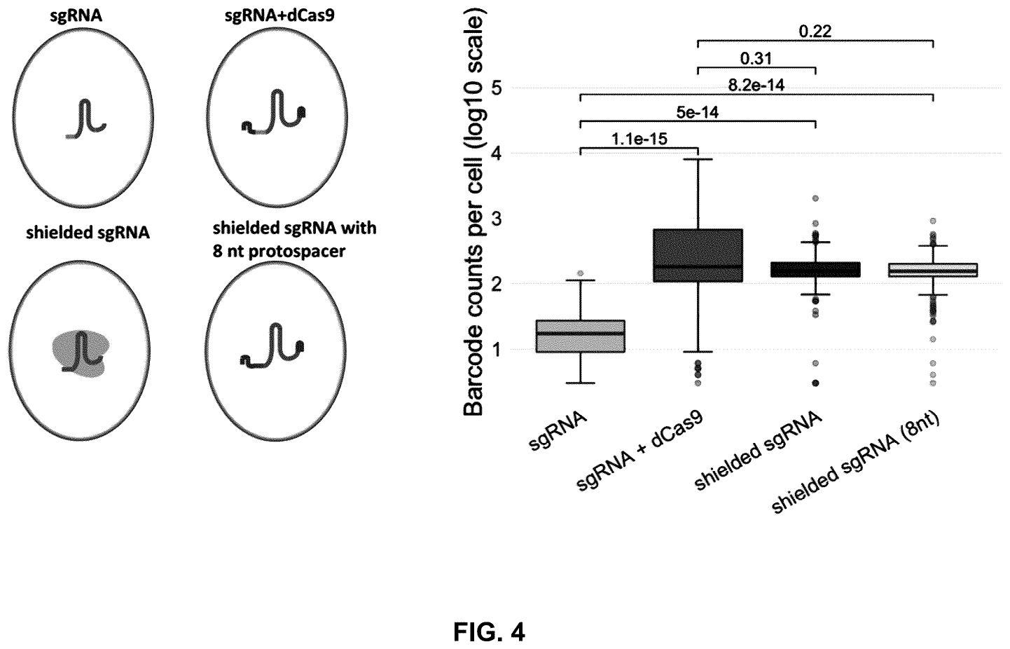

Next, K562 cells were transduced with lentivirus carrying the unmodified sgRNA, the shielded sgRNA with a 20 nucleotide protospacer, or the shielded sgRNA with an 8 nucleotide protospacer. K562 cells carrying both the sgRNA and dCas9 were included as a positive control. To validate the presence of the transcripts, a pilot experiment was performed that only mapped the presence of the sgRNAs. Significant improvement in barcode counts per cell was observed in the shielded sgRNA groups over the unmodified one, which was comparable to the group with dCas9. See FIG. 4.

### Example 4. Intracellular Barcoding with Shield Bacteriophage pRNA

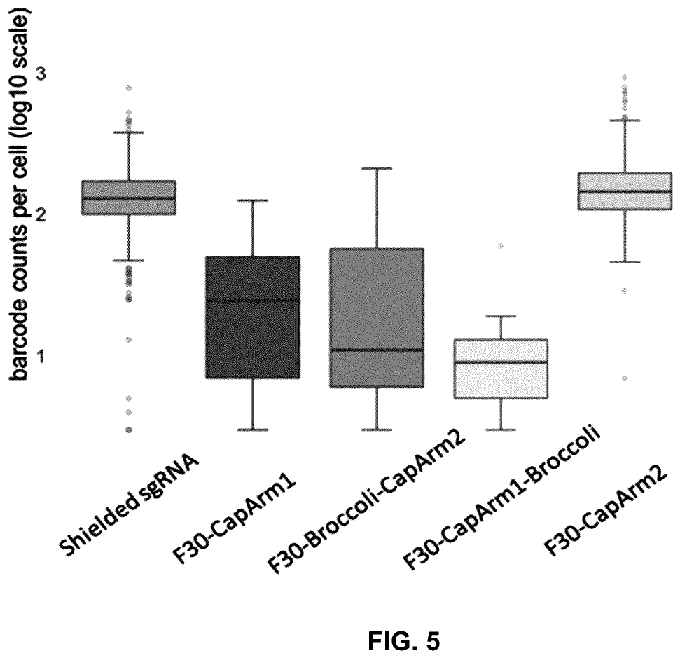

Additional small RNA sequences were tested to link the sample barcode and the capture sequence. The F29 RNA three-way junction motif and its derivative F30 were identified as scaffolds due to their superior scaffolding capacity and stability. F30-derived transcripts for sample barcoding were then designed along the same procedures as the sgRNA in Example 2. The 8-base barcode was placed at the beginning of the F30 scaffold, while the capture sequence was inserted into either the Arm 1 (termed as F30-CapArm1) or Arm 2 (F30-CapArm2). Broccoli, a fluorescent RNA aptamer, was also inserted into the other Arm of the constructs to create F30-CapArm1-Broccoli and F30-Broccoli-CapArm2. All four F30-derived constructs were incorporated into lentiviral vectors and transduced into K562 cells. Single-cell RNA-seq analysis showed that F30-derived small RNAs can be captured correctly, with the superior performance of F30-CapArm2 showing better or comparable levels as the shielded sgRNAs. See FIG. 5. These results suggested that when the capture sequence was integrated into the Arm2 of the F30 scaffold, it served as efficient sample barcodes for multiplexed single-cell RNA-seq.

### Example 5. Shield sgRNA and Shield Bacteriophage pRNAs are Effectively Captured Across Cell Types in Single-Cell RNA-Sequencing

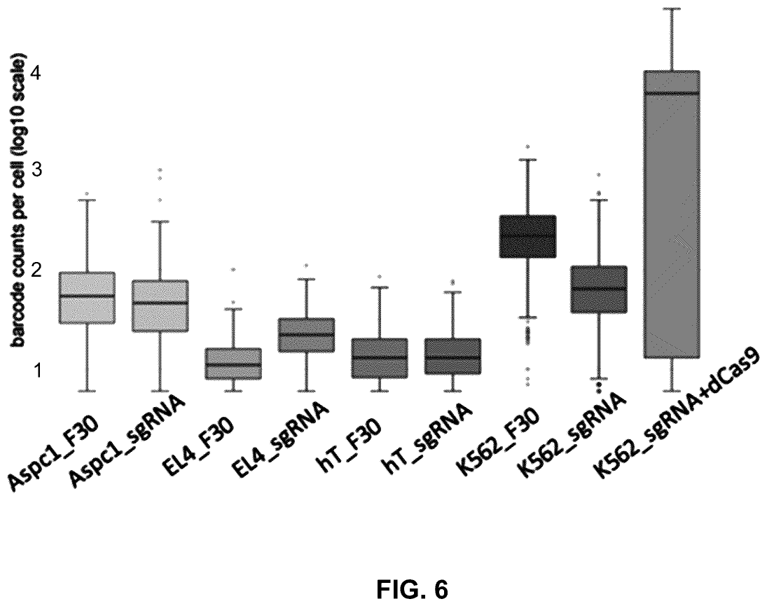

To test the performance of the SSN constructs, shielded sgRNAs and F30-derived small RNAs carrying corresponding sample barcodes were introduced to human primary T cells, K562 cells, Aspc-1 cells and mouse lymphoma EL4 cells. Additionally, K562 cells with constitutively expressed high-level dCas9 were used as both a positive control and a sgRNA-rich input. The cells were pooled and subjected to single-cell RNA-seq. See FIG. 6.

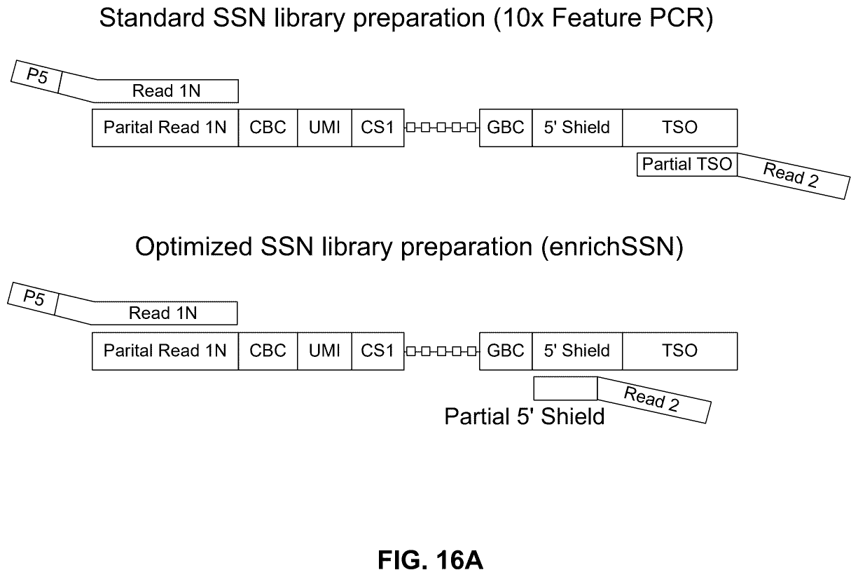

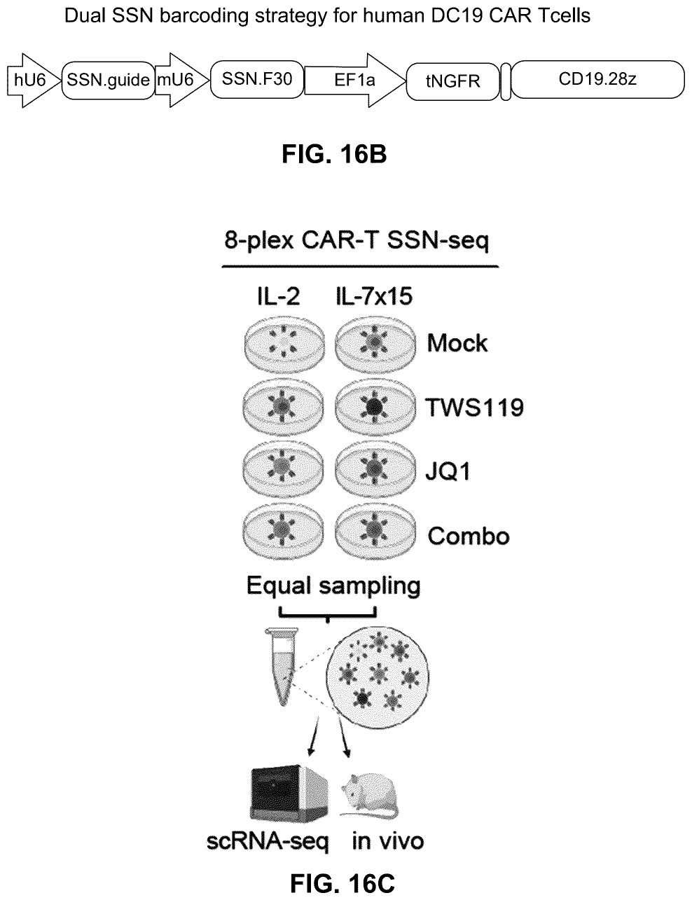

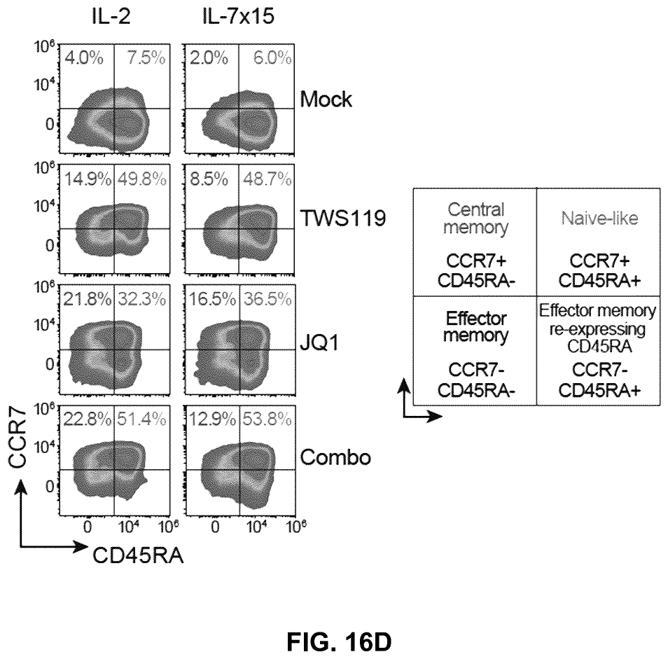

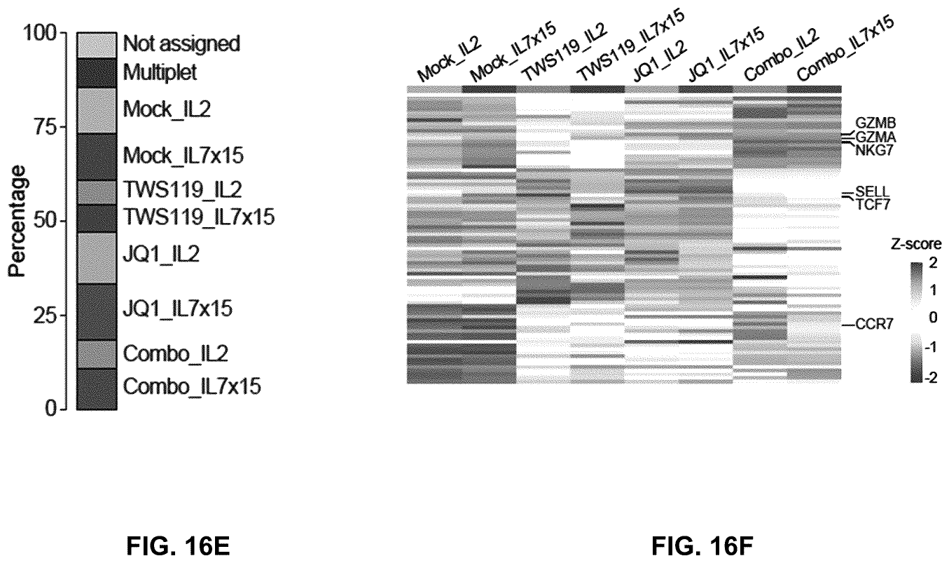

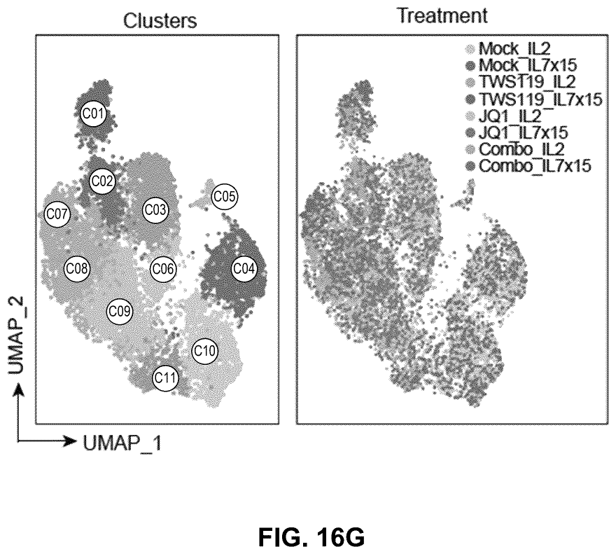

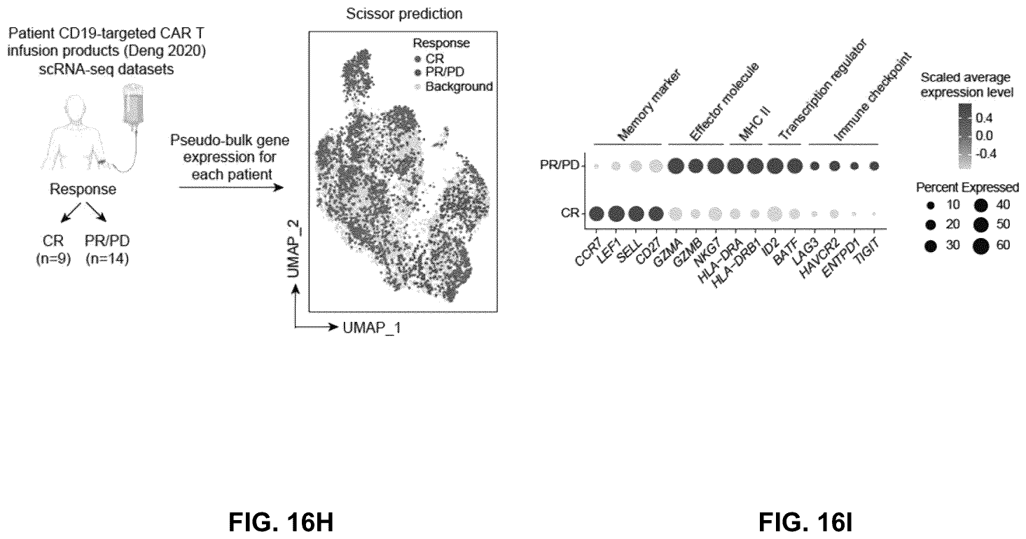

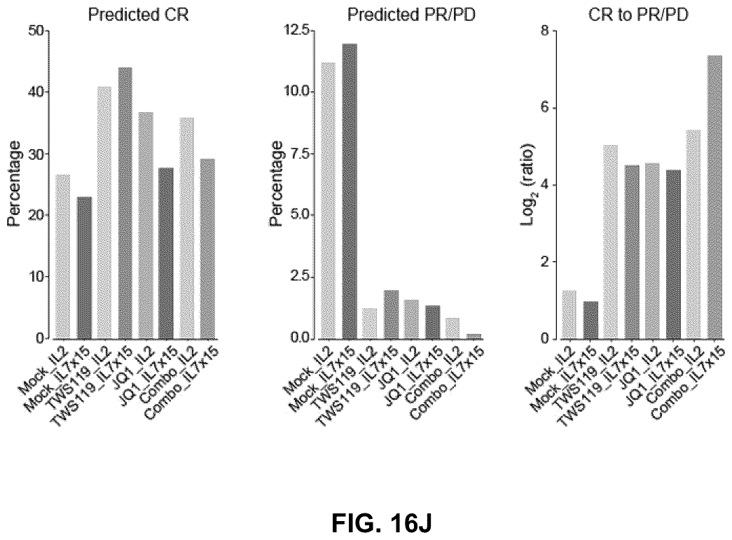

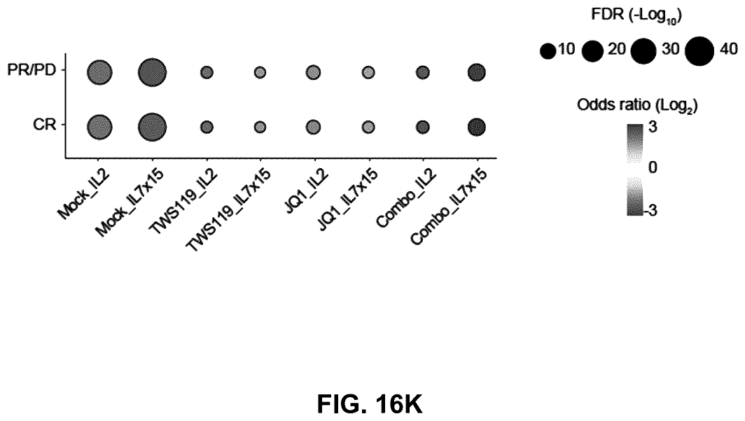

Pooled/sorted cells were processed for single-cell RNA sequencing at using Chromium Next GEM Single Cell 3′ Reagent Kit V3.1 (with feature barcoding technology for CRISPR screening), following the manufacturer's protocol. For enrichSSN amplification, a primer specific to the 5′ shield sequence was used (see FIG. 16A) and PCR was performed following 10× Genomics protocol for “Feature PCR” with 15 or 19 amplification cycles. Pooled libraries (the 3′ gene expression library and the sgRNA/SSN library, at a 4:1 ratio) were sequenced with a NovaSeq6000 SP-100 flow cell (Illumina) for each scRNA-seq experiment.

scRNA-seq data were preprocessed. In brief, gene expressing reads were aligned to the GRCh38 genome (human) and/or mm 10 genome (mouse) using the 10× Genomics Cell Ranger (version 6.0.2, 6.1.1 and 6.1.2), with the cellranger count pipeline. sgRNA/SSN counts were retrieved by cellranger count using the pattern containing unique group barcodes and constant sequences derived from the sgRNA/SSN scaffold. For SSN-seq in the 8-plex human CAR T cells, cellranger multi was additionally applied as a complementary method for group barcode assignment. To ensure a fair quantitative comparison, sgRNA/SSN UMI counts shown across different experiments were restricted to the raw output of cellranger count. Filtered feature-barcode matrices were further analyzed in R using Seurat (version 4.0.3). In general, genes detected in less than three cells and cells expressing <200 or >7,000 RNA features (genes) were filtered out. Low-quality cells containing a high percentage of mitochondrial reads were also excluded for subsequent analysis. Cells passing quality control were subjected to the standard Seurat workflow.

For example, for scRNA-seq dataset containing multiple cell types (related to FIG. 14 and FIG. 15), the standard Seurat pipeline was run for the first time to obtain general clustering for different cell types. Then, for each cell type, the percentage of mitochondrial UMI counts was examined. AsPC-1 cells containing more than 25% mitochondrial UMI counts were removed. For other cell types, the threshold was set to 15%. Then, the Seurat pipeline was run for the second time with the remaining cells for downstream analysis.

For human CAR T cells, gene expression was normalized and transformed using sctransform (version 0.3.2), with cell-cycle S-phase score and G2/M-phase score regressed. After Principal Component Analysis (PCA), UMAP reduction and clustering was performed to identify and visualize cell clusters.

For mixed-species cell types experiments, the UMAP reduction was performed with dimensions 1 to 10. For 8-plex CAR T experiments, the UMAP reduction was performed with dimensions 1 to 30.

For the pilot scRNA-seq experiment using standard sgRNAs (related to FIG. 14C), cells identified as doublets by DoubletFinder were removed. Marker genes for each cell cluster were determined using the FindAllMarkers function in Seurat where genes detected in at least 10% or 25% of cells were tested. Differentially expressed genes (DEGs) between two groups were identified by FindMarkers function in Seurat. R package EnhancedVolcano (version 1.12) was used to generate volcano plots for DEGs. CD4/CD8 T cell calling, heat map showing unique marker genes and contour UMAP plots were generated.

To characterize the distribution of different treatment groups among indicated cell clusters, Fisher's exact test was applied. Odds ratios were calculated to indicate preferences, and the false discovery rate (FDR) was calculated by the Benjamini-Hochberg procedure. Single-cell gene set enrichment was performed using AUCell (version 1.16) on curated gene sets from the MSigDB Immunologic Signatures database (gsea-msigdb.org/gsea/msigdb).

All sample barcodes were successfully retrieved for different cell types, with the recovery rate ranging from ˜40% to ˜90%. The K562-dCas9 groups showed the highest barcodes count per cell. Notably, the reads from F30-derived small RNAs were comparable to shielded sgRNAs in barcoding human T cells and Aspc-1 cells, while lower in EL4 cells and higher in K562 cells. These results provided support that shielded sgRNAs or F30-derived small RNAs alone were capable of labeling different cell types, allowing multiplexed single-cell RNA-seq in a high-throughput form. Considering the variation of the guide version and the F30 version in different cells, these two types of SSN-seq offered flexibility based on user-defined criteria, especially when higher reads were needed in certain experiments.

### Example 6. Shield sgRNAs Used to Intracellularly Barcode CAR-T Cells

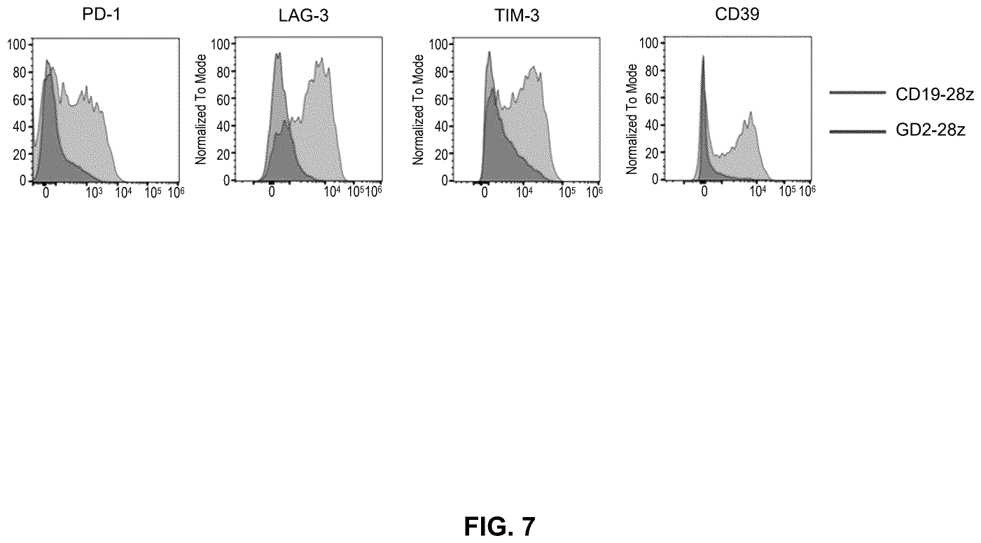

To study Chimeric Antigen Receptor (CAR) T cell exhaustion, a CAR was transduced with lentiviral vectors into human T cells along with corresponding shielded sgRNA barcodes. The CAR incorporated the CD19-specific FCM63 scFv or the disialoganglioside (GD2)-specific 14g2a-E101K scFv16, with CD28 and CD35 signaling domains (CD19-28z or GD2-28z). Flow cytometry confirmed that the GD2-28z CAR-T cells showed signs of exhaustion signatures including elevated immune checkpoint markers PD-1, LAG-3, TIM-3 and CD39. See FIG. 7.

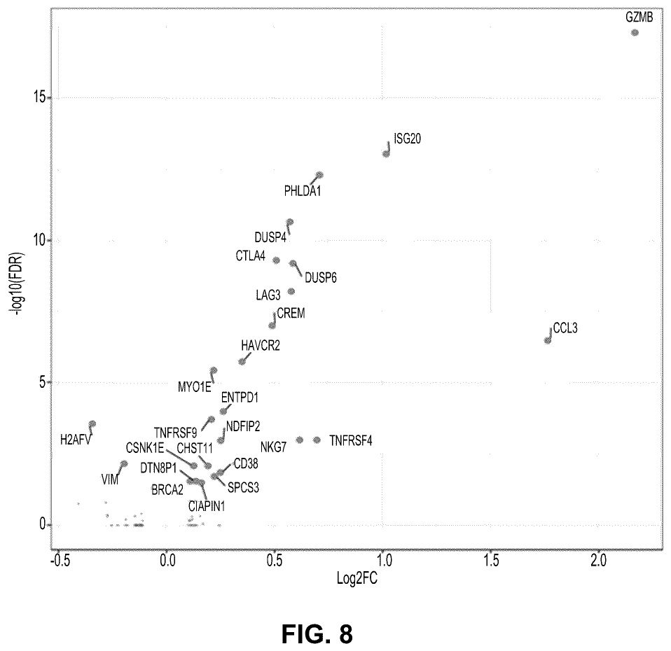

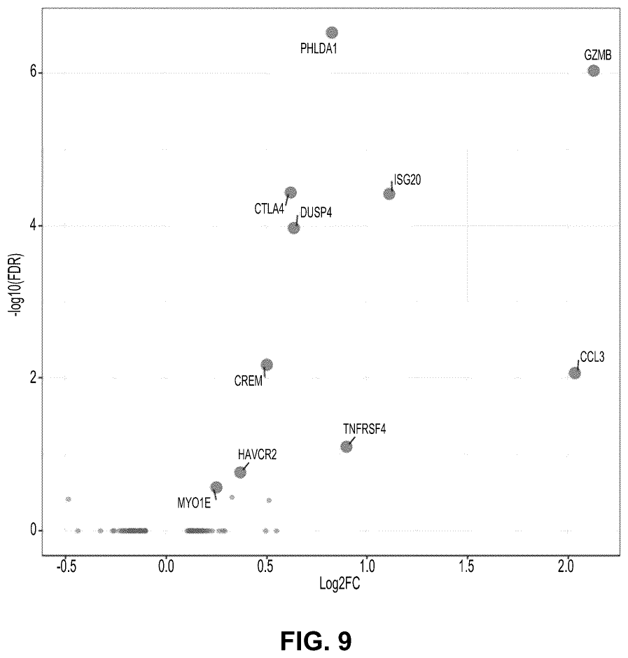

The CAR T cells were then pooled and subjected to single-cell RNA sequencing and were demultiplexed for analysis. ˜50% of the cells were successfully assigned as CAR-T cells carrying shielded sgRNA barcodes for CD19-28z or GD2-28z. Comparing the two CAR-T groups revealed elevated exhaustion signatures in T cells with GD2-28z. See FIG. 8. The top 10 most significant changed genes (FIG. 9) included activation-associated genes (GZMB, ISG20, TNFRSF4), activation-induced cell death (AICD)-related genes (PHLDA1), negative regulators of proliferation (DUSP4) and immune checkpoint inhibitors (CTLA4, HAVCR2), further highlighting the exhaustion status of GD2-28z CAR-T cells. See FIG. 9.

### Example 7. SSN-Seq Enables Scalable Sample Multiplexing

The capacity of SSNs carrying unique barcodes to demultiplex scRNA-seq samples was evaluated using a panel of cells including K562, AsPC-1, EL4 (mouse lymphoma), and human primary T cells (FIG. 15A). For K562 cells, additional modifications to SSN.guide and SSN.F30 sequences were tested to assess impact on the performance of scRNA-seq and potential perturbative effects on the transcriptome (FIG. 19A). Standard sgRNAs (STD.guide) with or without Cas9 were included as controls. Human primary T cells were transduced to express chimeric antigen receptors (CARs) incorporating CD28 and CD35 signaling domains and single-chain variable fragments (scFv) targeting CD19 (CD19.28z) or GD2 with demonstrating higher affinity E101K mutation (HA.GD2.28z). In contrast to CD19.28z, expression of HA.GD2.28z in T cells results in tonic signaling via antigen-independent aggregation, which triggers exhaustion phenotype characterized by elevated expression of immune checkpoint markers PD-1, LAG-3, TIM-3 and CD39 as confirmed in (FIG. 15B). Early T cell exhaustion is a primary factor limiting anti-tumor efficacy of CAR T cells. Therefore, CD19.28z and HA.GD2.28z were utilized to model distinct transcriptional signatures to demonstrate demultiplexing accuracy of SSNs. In total 20 barcoded groups (G01-G20) of cells (FIG. 15A) were pooled at an equal number ratio and subjected to scRNA-seq following 10× Genomics single cell 3′ gene expression with feature barcoding technology workflow. All 20 groups of barcodes were successfully retrieved, and analyses revealed (FIG. 15C) that alterations in the length or sequence content had no effect (G03-G07) but reverse orientation of the U6 promoter reduced SSN recovery (G08-G09) and that the UMI counts from SSN.F30 are comparable to SSN.guide in the same cell type (e.g. G13 vs G14). The recovered SSN barcodes showed a high correct assignment rate to corresponding cell types (˜94% for K562, ˜86% for AsPC-1, ˜61% for EL4, ˜38% for human primary T, FIGS. 15D-15E). Notably, correlation analysis of SSN-barcoded groups within the same cell type showed no signs of transcriptome perturbation (FIG. 19B). Analysis of human primary T cells and CD19.28z and HA.GD2.28z CAR T cells revealed that cells group into three sub-clusters C1-C3 (FIG. 16F). Control T cells and CD19.28z CAR T cells showed a similar enrichment of C1 and C2 sub-clusters. In contrast, HA.GD2.CAR T cells were dominated by the C3 sub-cluster (FIG. 16G). Of note, distinct C3 transcriptional profile is consistent with hallmarks of T cell exhaustion and includes elevated expression of immune checkpoints (LAG3, ENTPD1, TIGIT), exhaustion marker PHLDA1 and effector molecules (IFNG, GZMB) (FIG. 16H), consistent with exhaustion phenotype of HA.GD2.28z CAR T cells. These results showed that enhanced SSN.guide and SSN.F30 stability in the absence of Cas9 results in superior barcoding performance in multiplexed scRNA-seq of various cell types including human primary T cells.

SSN-seq is a scalable method that enables any number of barcoded samples to be multiplexed and can be utilized for tracking clonal heterogeneity and evolution of cell lineages, e.g., to advance our understanding of how diverse tumor and immune programs drive therapy resistance and inform novel therapeutic strategies. Considering the flexibility for the variable region, a library of SSNs can be generated as unique labels, to enable individual cell clonal tracking. SSN-seq can leverage simultaneous longitudinal lineage-tracing and transcriptional profiling to enable the identification of rare cell states like persistent cancer cells in minimal residual disease. SSN-seq is also a versatile approach because SSN barcodes are expressed using ubiquitous U6 promoters which maintain robust small RNAs expression across a large variety of cells and tissues in vitro and in vivo. The modular design and compact size of SSN-expressing cassettes (U6 promoter and SSN barcode: <450 bp) warrant compatibility with viral and non-viral vectors without compromising titer or knock-in efficiency. Moreover, since transcripts driven by U6 promoters tend to accumulate in the nucleus, SSN-seq can be utilized to couple transcriptome and chromatin accessibility (ATAC-seq) profiling in longitudinal studies to comprehensively map cellular states at single-cell level. SSN stability permits the use of standard sample preservation strategies such as flash-freezing and fixation, which further streamlines workflows for complicated experimental design. Therefore, the SSN-seq approach fills a recognized void in the field and is readily compatible with standard high-throughput droplet microfluidic platforms such as the 10× Chromium and computational analysis tool, which should facilitate adaptation of the method. SSN-seq will empower researchers to study transcriptional state changes in various challenging in vitro and in vivo models.

### Example 8. Modification of SSN-Seq for Human Primary T Cells Labeling

Human peripheral blood mononuclear cells (PBMCs) from healthy donors were purchased from Stem Cell Technologies. PBMCs were activated with anti-human CD3/CD28 Dynabeads (Gibco) at a 1:1 bead-to-cell ratio and were cultured in RPMI 1640 medium containing 10% FBS, 10 mM HEPES, 2 mM L-glutamine, 1 mM sodium pyruvate, 1×MEM non-essential amino acids (Gibco). The T cell culture media were further supplemented with 100 IU/ml recombinant human IL-2 (Peprotech) or recombinant human IL-7 and IL-15 at 5 ng/ml each (PeproTech). Mouse T cells were isolated from dissociated spleens from C57BL/6 mice, using Easy Sep™ Mouse CD8+ T Cell Isolation Kit (Stemcell Technologies). T cells were activated with anti-mouse CD3/CD28 Dynabeads (Gibco) at a 1:1 bead-to-cell ratio and were cultured in a similar medium for the human T cells except with the additional 55 μM β-mercaptoethanol and supplemented with 10 ng/ml recombinant mouse IL-2 (Peprotech).

The enhanced stability of SSNs compared to standard sgRNA in the absence of Cas9 increased recovery of barcodes and their correct assignment into respective cell type groups, albeit to a lesser extent for human primary T cells (20% vs. 38%, see FIG. 14D and FIG. 15E). Correspondingly, the UMI counts of SSNs in primary T cells were significantly lower than in immortalized cell lines (FIG. 15C), likely resulting in suboptimal performance. Consistent with the low SSNs expression, substantially less total mRNA UMI counts was observed in primary T cells in contrast to immortalized lines (FIG. 19C-19D), indicating a limited global transcript abundance. Therefore, additional strategies were sought to further improve the SSN recovery for human primary T cells.

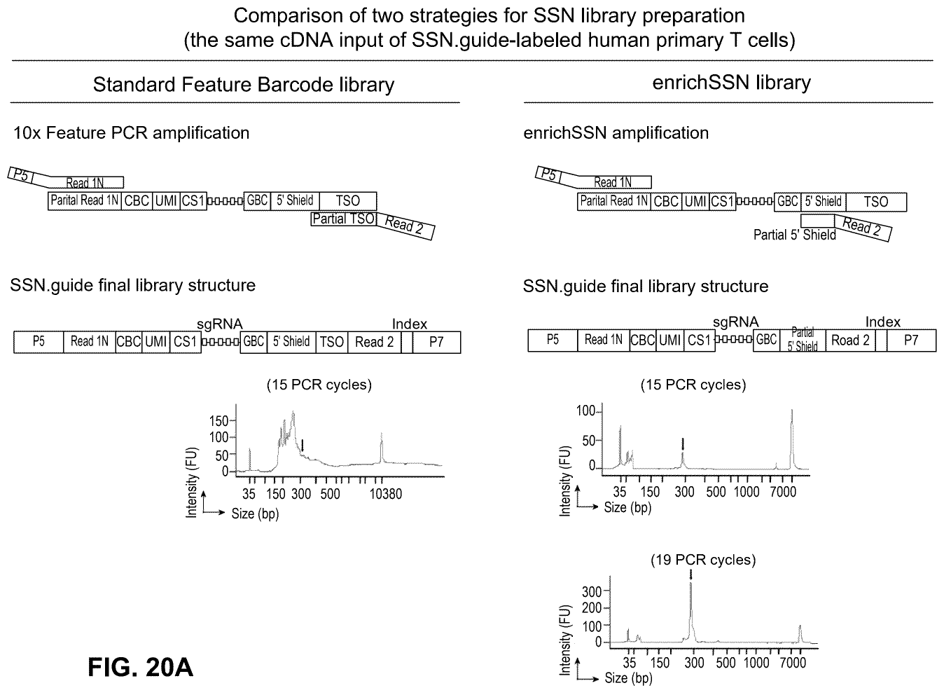

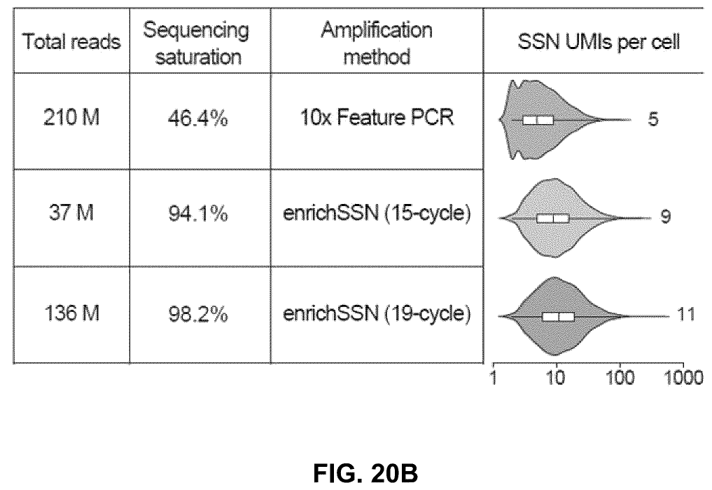

In the preparation of SSN sequencing libraries, a standard protocol for direct-capture compatible sgRNAs amplification utilizes the template-switch oligo (TSO) as a PCR handle (FIG. 16A) which results in contamination with multiple off-target fragments (FIG. 20A). To increase the specificity of SSN barcode amplification during sequencing libraries generation, the 5′ shield sequence-specific primer was designed (FIG. 16A). Substituting the TSO primer with the 5′ shield-specific primer enriched precision of SSN barcode amplification (denoted as “enrichSSN”) (FIG. 20A). The precise amplification led to a higher level of sequencing saturation and recovered more SSN UMIs at the cost of fewer reads than standard protocols (FIG. 20B). It was anticipated that the enrichSSN amplification strategy would improve the SSN-seq efficiency and warrant utility for subsequent validation experiments using human primary T cells.

### Example 9. SSN-Seq Profiling of Pooled Human CAR T Cells Ex Vivo

To demonstrate the capacity of SSN for barcoding multiple samples in pooled scRNA-seq, the impact of various CAR T cell manufacture conditions on transcriptional profiles both ex vivo and in vivo were evaluated. To that end, CD19 CAR containing the CD28 intracellular domain (CD19.28z) based on the clinically approved Axicabtagene ciloleucel (axi-cel) CAR T cell therapy for large B cell lymphoma was utilized. Clinically, CD19.28z cells yield robust tumor clearance and tolerance of lower antigen levels, but lack durable persistence. The persistence of CAR T cells is correlated with the durability of clinical remissions in cancer patients. Thus, different approaches have been developed to enhance persistence of CAR-T cells, for instance enrichment of specific T cell subsets (e.g. CD4+/CD8+ T cells with naïve/stem cell-like properties), optimized T cell manufacturing conditions (e.g. IL2, IL7, IL15 cytokines), modifications of CAR designs (e.g. CD28, 4-1BB costimulatory domains), and combination therapies (e.g. PD-1).

The following antibodies were used for cell surface staining: CD45-FITC (clone 2D1), NGFR-PE (clone ME20.4), NGFR-APC (clone ME20.4), CD39-APC-Cy7 (clone A1), TruStain FcX™ (anti-mouse CD16/32) purchased from BioLegend; PD-1-APC (clone J105, eBioscience), LAG-3-PE-Cy7 (clone 3DS223H, eBioscience); TIM-3-BV421 (clone 7D3, BD Biosciences). Dead cells were excluded by 7-AAD (ThermoFisher) staining. Cells were analyzed using Attune NxT flow cytometer (ThermoFisher) and FlowJo v10 software (FlowJo, LLC). Fluorescence-activated cell sorting (FACS) was performed using SH800 Cell Sorter (Sony Biotechnology).

8-plex CAR T SSN-seg ex vivo (infusion product). Twenty-four hours after activation, human primary T cells were transduced with lentiviral vectors (see FIG. 16B). Transduced T cells were cultured in the presence of 5 μM TWS119, 0.15 μM JQ1, the combination of TWS119 and JQ1 (Combo), or DMSO (Mock) controls. CAR T cells in four treatment groups were expanded in culture media supplemented with IL2 or IL7 plus IL15 (denoted as IL7×15) cytokines. Culture media containing small molecule inhibitors and cytokines were replenished every other day. CAR T cells were maintained at a concentration of 0.5 to 1×10⁶ cells per ml. On day 8, expression of T cell memory makers (CCR7 and CD45RA) was evaluated in all treatment groups using flow cytometry. Then, CAR T cells in all groups were counted and pooled at equal ratios. To enrich CAR⁺ population a cell surface reporter NGFR⁺ was utilized to FACS purify CAR T cells before scRNA-seq analysis. Aliquots of the pooled samples were also injected into tumor mice. Remaining CAR T cells were cryopreserved in CryoStor CS10 media (Stem Cell Technologies).

8-plex CAR T SSN-seq in vivo. Four days before CAR T administration, 1×10⁶ NALM6-Luc2 leukemia cells were intravenously injected into eight-week-old NOD/scid/IL2rγ^{-/-} (NSG) mice. On the same day when the 8-plex CAR T infusion product was prepared for scRNA-seq, 2×10⁶ pooled CAR T cells were injected into each tumor mice. Three weeks later, the mice were rechallenged with 1×10⁶ NALM6-Luc2 tumor cells injection. To monitor tumor burden, mice were injected intraperitoneally with 150 mg/kg D-Luciferin (Sigma-Aldrich). Bioluminescent images were acquired in an AMI HTX bioluminescence imaging system (Spectral Instruments Imaging). Six weeks after CAR T administration (three weeks after the tumor rechallenge), spleens from three tumor-free mice were harvested. Aliquots of splenocytes from each mouse were first examined by flow cytometry to estimate the CD45⁺NGFR⁺ proportion for each spleen. Then splenocytes were pooled at a ratio to achieve approximately equal sampling of CD45⁺NGFR⁺ cells for each mouse. The pooled sample was further FACS-sorted to obtain CD45⁺NGFR⁺ CAR T cells, and was subjected to scRNA-seq.

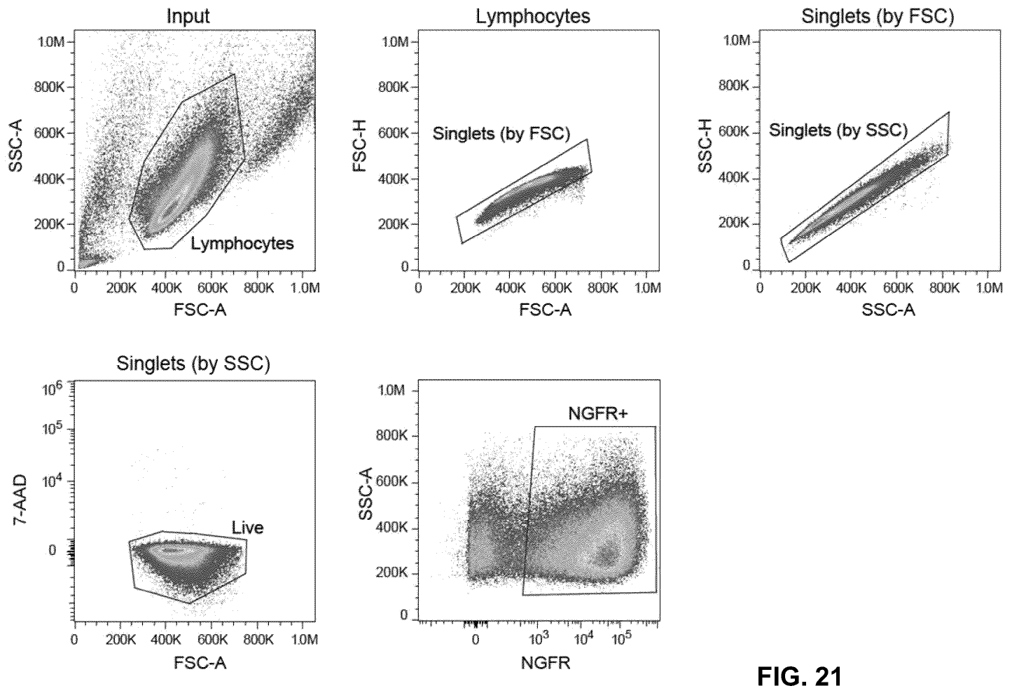

SSN barcoding was used to directly compare various experimental conditions on CAR T cells transcriptional state, including (i) GSK3ß inhibitor (TWS119) treatment to mimic WNT signaling activation shown to promote CD8+T naïve/stem cell-like state; (ii) BET proteins inhibitor (JQ1) treatment reported to support functional stem cell-like and central memory CD8+ T cells properties and superior persistence and antitumor effects; (iii) CAR T expansion in the presence of the cytokine IL2 (standard conditions) or IL7 and IL15 (denoted as IL7×15), demonstrated to preserve better memory phenotype of T cells. Finally, the combination of these modalities (FIG. 16C) was tested. To directly evaluate SSN.guide and SSN.F30 barcoding strategies lentiviral vectors were generated to express both SSN labels using human and mouse U6 promoters and NGFR-tagged CD19.28z (FIG. 16B). Expression of CAR was confirmed by flow cytometry (NGFR+, see FIG. 21).

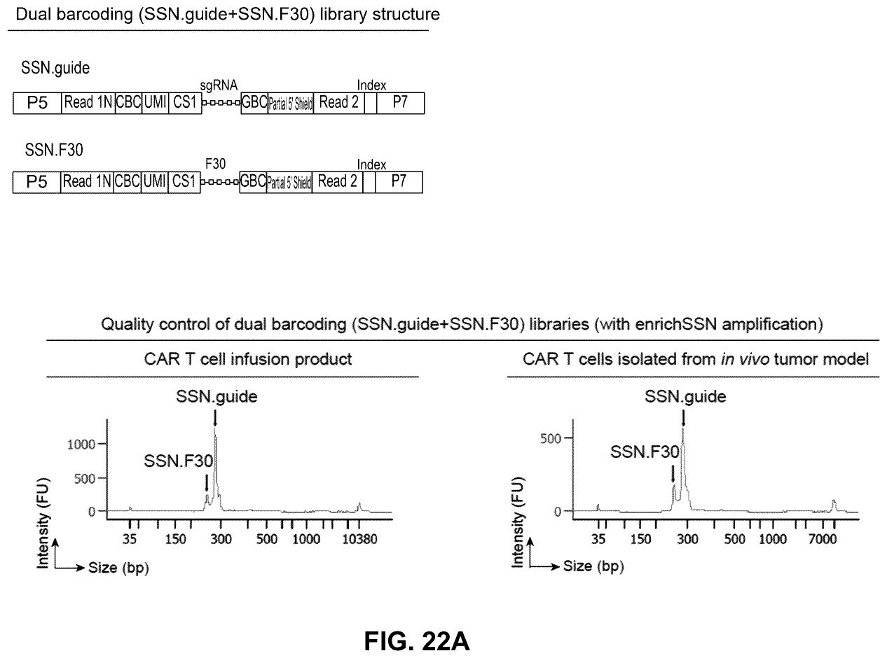

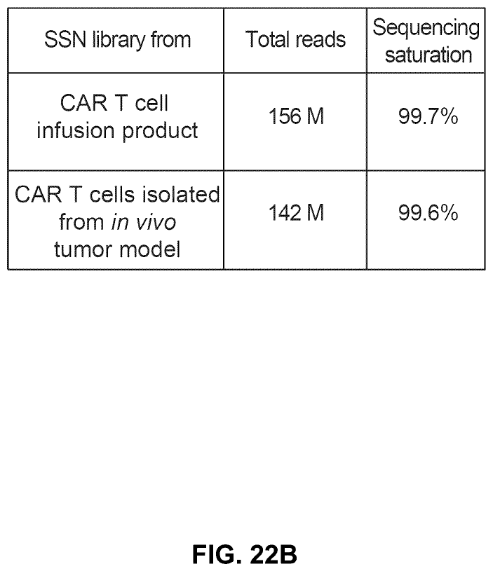

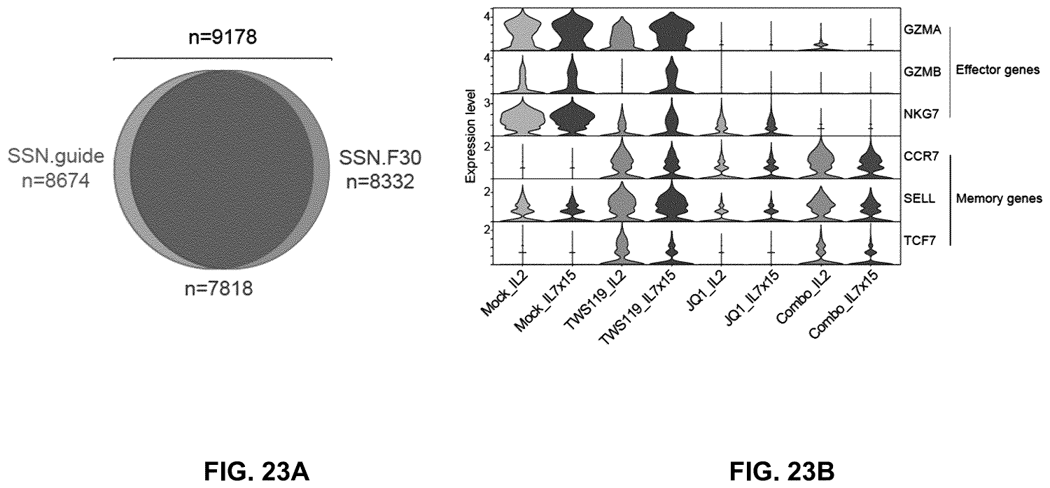

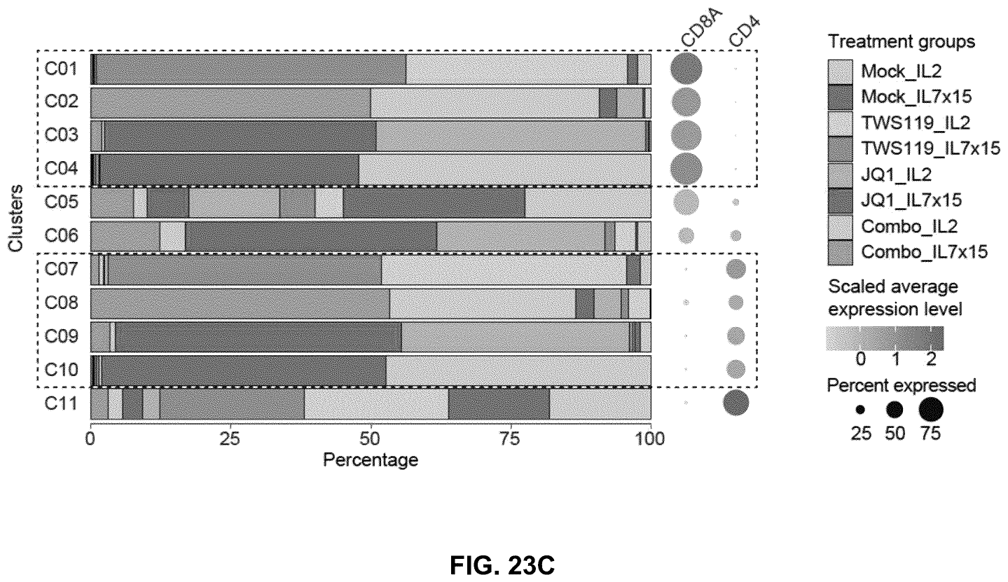

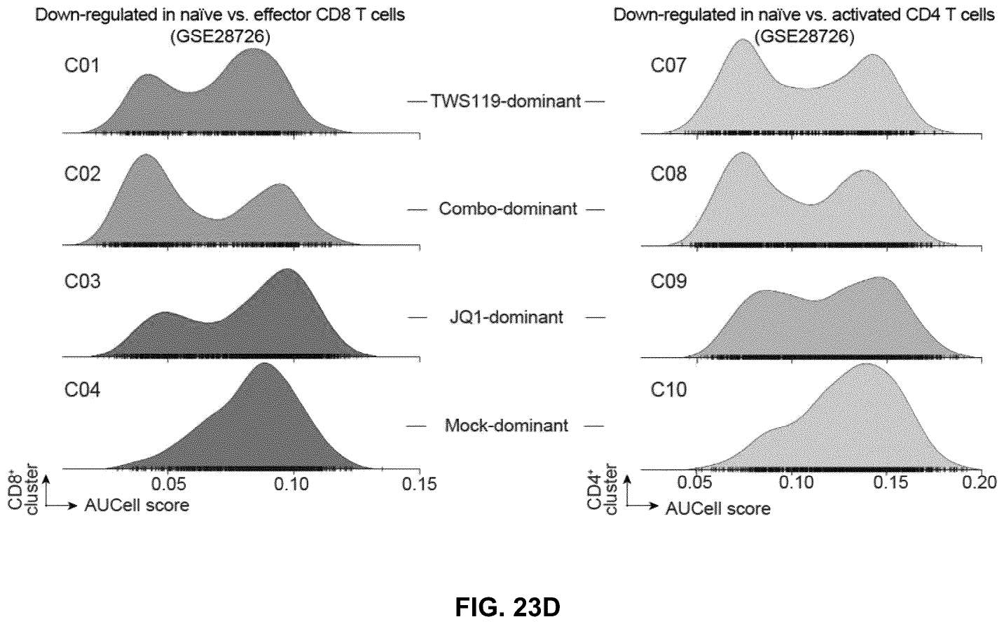

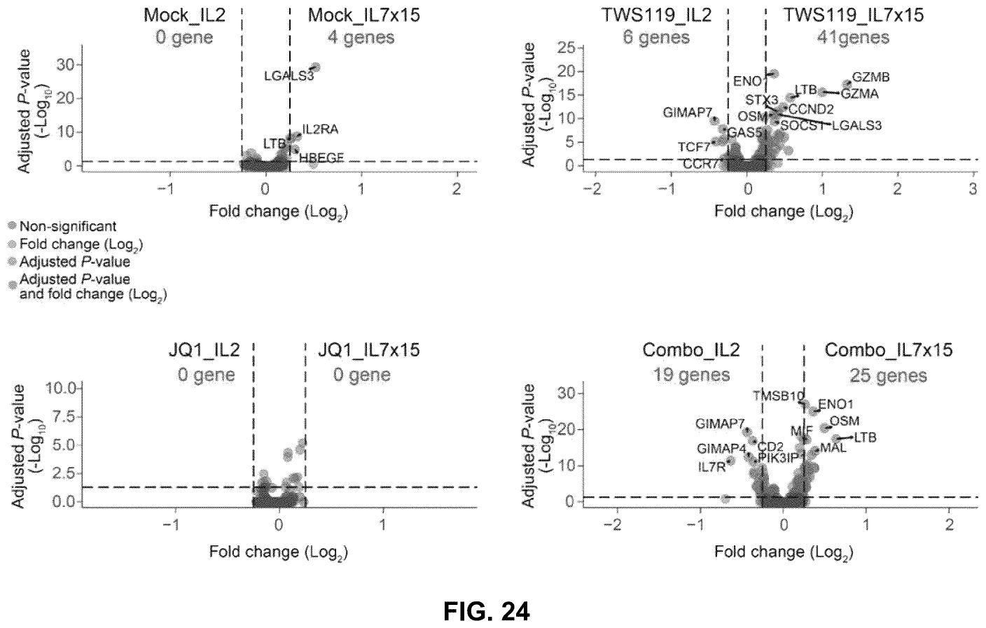

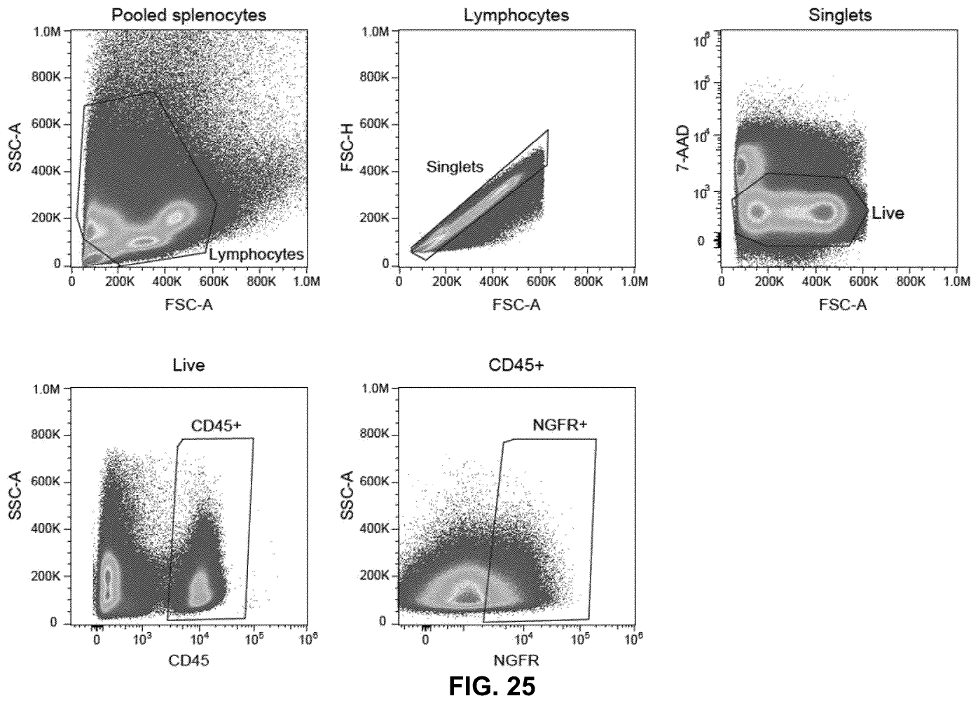

Consistent with previous studies CAR T cells showed enrichment of naïve and central memory markers upon TWS119 and/or JQ1 treatment (FIG. 16D). NGFR+ cells from each testament group were then pooled at equal number ratio and subjected to scRNA-seq following standard 10× Genomics single cell 3′ gene expression with feature barcoding technology workflow. SSN.guide and SSN.F30 barcodes were successfully amplified using the optimized enrichSSN library preparation protocol (see FIG. 22A). In total 9,854 cells were successfully profiled with over 93% assigned with at least one unique SSN (FIG. 16E). Detection rates for SSN.guide and SSN.F30 (88% and 84%, respectively) indicate the similar performance of both barcoding strategies (FIG. 23A). Transcriptome analyses revealed that the eight groups showed enrichment of either memory or effector markers (FIG. 16F), consistent with the flow cytometry results (see FIG. 16D). Top enriched genes in the two mock-treated groups were effector/cytotoxicity molecules, including GZMA, GZMB and NKG7 (FIG. 16F and FIG. 23B). CAR T cells treated with TWS119, JQ1 and TWS119+JQ1 (Combo) were enriched in memory-related genes like CCR7, SELL (encoding CD62L), and TCF7 (FIG. 16F and FIG. 23B). UMAP dimensional reduction followed by unsupervised clustering revealed that cells representing eight treatment modalities cluster into ten distinct transcriptome profiles (FIG. 16G). Notably, CD8+ clusters C01-C04 and CD4+ clusters C07-C10 (comprising ˜91% of all cells) contained cells mostly from the same drug treatment condition (FIG. 23C). Gene set enrichment analysis identified significant downregulation of naïve and elevation of activation/effector T cell signatures in clusters C04 and C10 grouping the mock-treated cells (FIG. 23D). Of note, IL2 vs. IL7×15 cytokines supplementation alone or in combination with other treatment conditions led to modest changes in global transcriptomes (FIG. 16F), and only a few significant differentially expressed genes were identified (FIG. 24).

Next, to associate the ex vivo CAR T cell transcriptional states with clinical outcomes, a recently developed computational algorithm Scissor and pseudo-bulk RNA profiles of axi-cel (CD19.28z) CAR T cell infusion products from large B cell lymphoma (LBCL) patients with corresponding patient response information was applied (FIG. 16H).

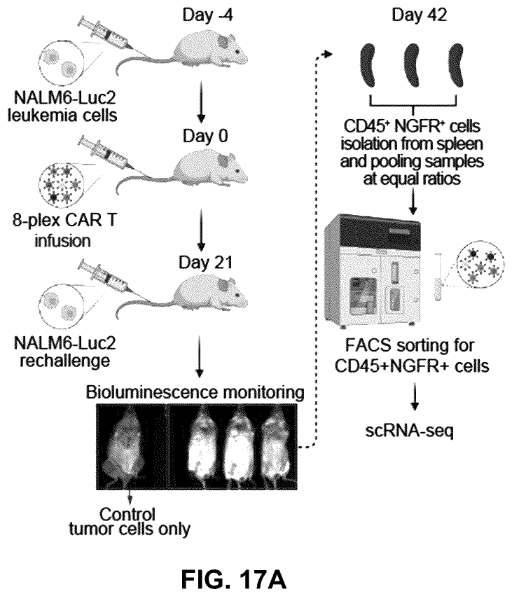

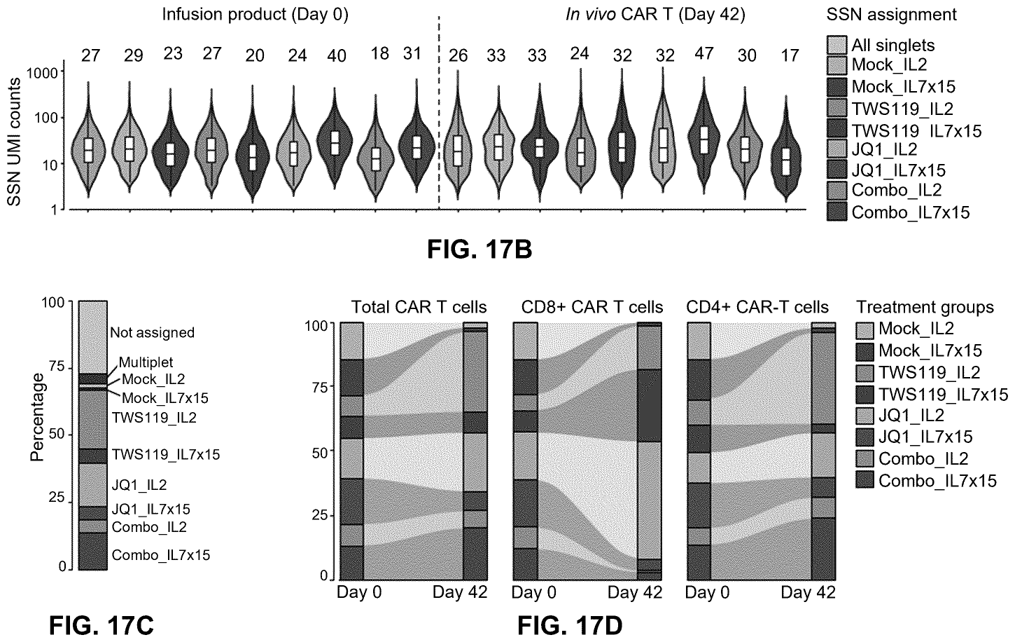

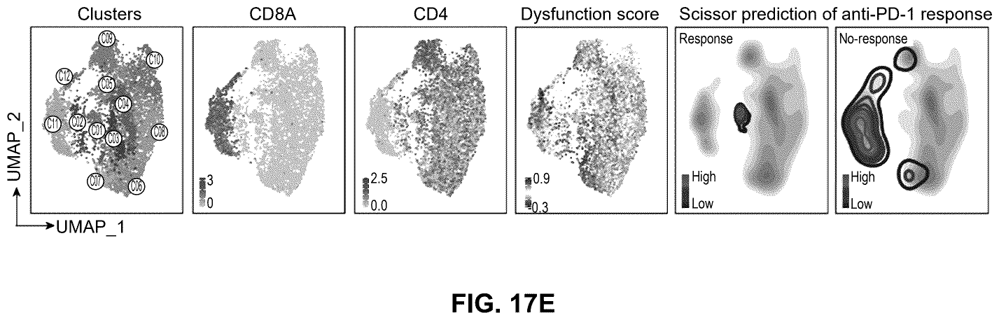

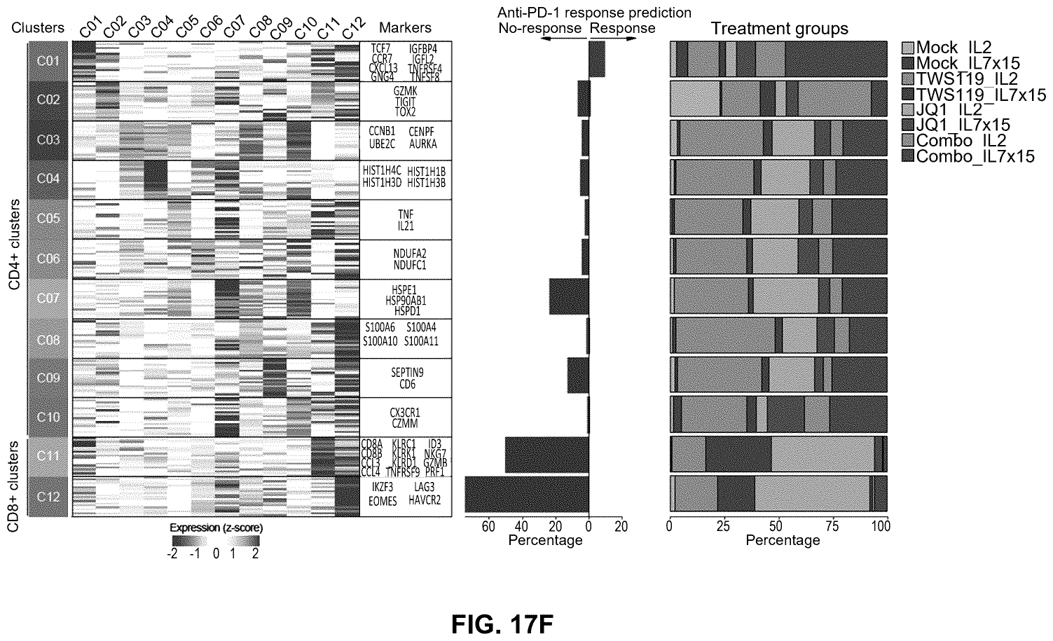

Scissor algorithm was utilized to predict the association of cell populations from scRNA-seq with clinical phenotype using bulk RNA-seq data (github.com/sunduanchen/Scissor/). In this study, “pseudo-bulk” gene expression matrix was generated by averaging the normalized gene expression from T cells obtained from indicated scRNA-seq datasets. The resulting expression matrix and the corresponding clinical response phenotype were used as the input together with the 8-plex CAR T scRNA-seq data for Scissor prediction. “family=‘binomial’” in Scissor was applied to select the clinical response-associated cells. Scissor prediction generated Scissor+ cells (i.e., response) and Scissor-cells (i.e., no-response). The remaining unassigned cells were labeled as “Background”. For Scissor predictions for the CAR T infusion product (8-plex CAR T group ex vivo), there was analysis of publicly available single-cell RNA sequencing datasets of axicabtagene ciloleucel (axi-cel) anti-CD19 CAR T cell infusion products (GEO; accession number: GSE150992) 19 from 9 patients with complete response (CR), one patient with partial response (PR), 13 patients with progressive disease (PD), and one patient not evaluable (NE). CD8+ and CD4+ T cells were extracted from each CR, PR/PD patient and the average normalized gene expression matrix was used as “pseudo-bulk” input for Scissor. The results reported (see FIG. 16H) are based on Scissor predictions from CD8+ T cells of the patients, as CD4+ input returned all background cells. For Scissor predictions for the CAR T isolated from spleens (8-plex CAR T group in vivo), scRNA-seq datasets (GEO; accession number GSE120575) of CD45+ intratumoral immune cells obtained from human melanoma patients were analyzed. The expression matrix of CD8+ and CD4+ T cells at baseline (before treatment) was extracted from patients who received subsequent anti-PD1 or anti-PD1 plus anti-CTLA4 therapies. The procedure generated profiles of 8 patients with “response” and 9 patients with “no-response”. The results reported (see FIGS. 17E-17F) are based on predictions from both CD8+ and CD4+ T cells of the patients. The analyzed cells were associated with “response” or “no-response” phenotype only when Scissor predictions were consistent from the CD8+ and CD4+ inputs.

In line with the previous findings, CAR T cells predicted to complete response (CR) had higher expression of T cell memory markers (CCR7, LEF1, SELL, CD27) and lower expression of effector proteins (GZMA, GZMB, NKG7), MHC II molecules (HLA-DRA, HLA-DRB1), transcription regulator ID2 and BATF, and immune checkpoints (LAG3, HAVCR2, ENTPD1, TIGIT) than their counterparts categorized as partial response or progressive disease (PR/PD) (FIG. 17I). Notably, TWS119, JQ1 and Combo-treated CAR T cells exhibited enrichment of transcriptional profiles predicted as CR. In contrast, the cells in the control groups are more frequently associated with PR/PD, leading to a significant difference in the ratio of CR to PR/PD cells (FIGS. 17J-17K). These observations indicate that CAR T cell manufacturing conditions have significant effects on the cell transcriptional profile associated with durability of clinical response and demonstrate the accuracy and efficiency of SSN-seq barcoding for sample multiplexing.

### Example 10. Shielded Small Nucleotides May be Used to Barcode CAR-T Cells for In Vivo Experiments

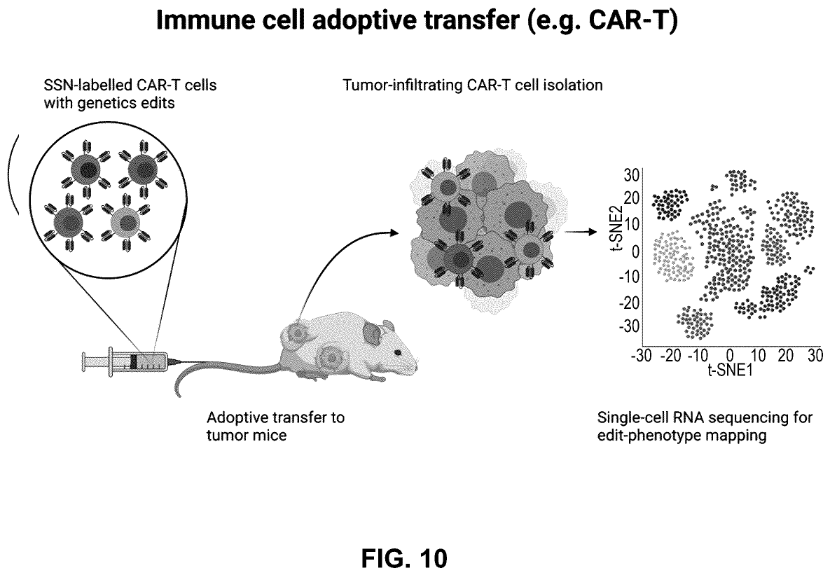

CAR-T cells were analyzed by multiplexed single-cell RNA sequencing using Shielded Small Nucleotides (SSNs). CAR-T cells comprising different genetic edits were labelled by SSNs, then transferred into tumors in mice. After a sufficient time period, the tumor was extracted from the mouse and tumor-infiltrating CAR-T cells are isolated. Next, the CAR-T cells were subjected to single-cell RNA sequencing analysis to determine the phenotypes for the CAR-T cells with different genetic edits. See FIG. 10. The shielded sgRNA barcode labelling allowed the sample to be demultiplexed.

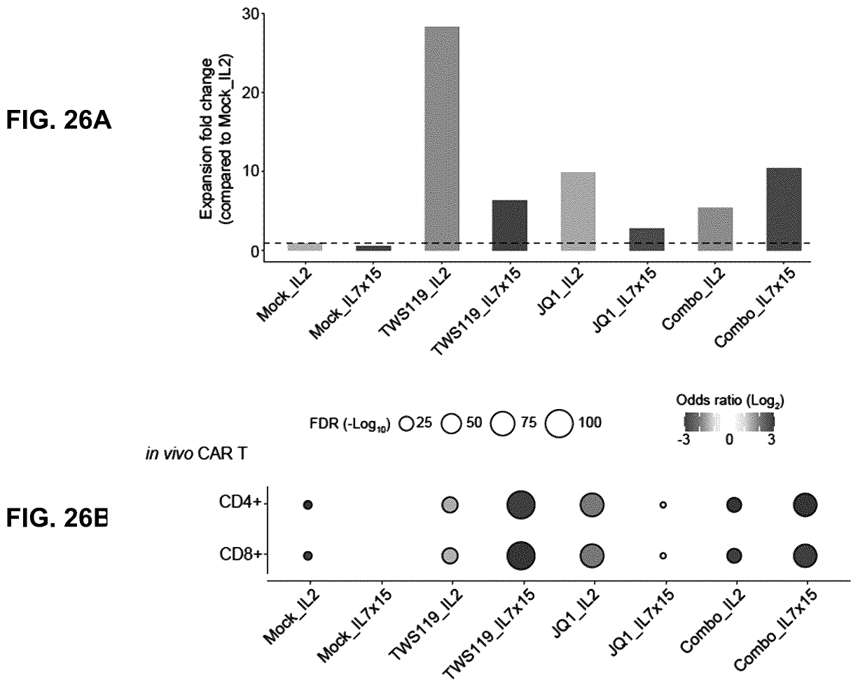

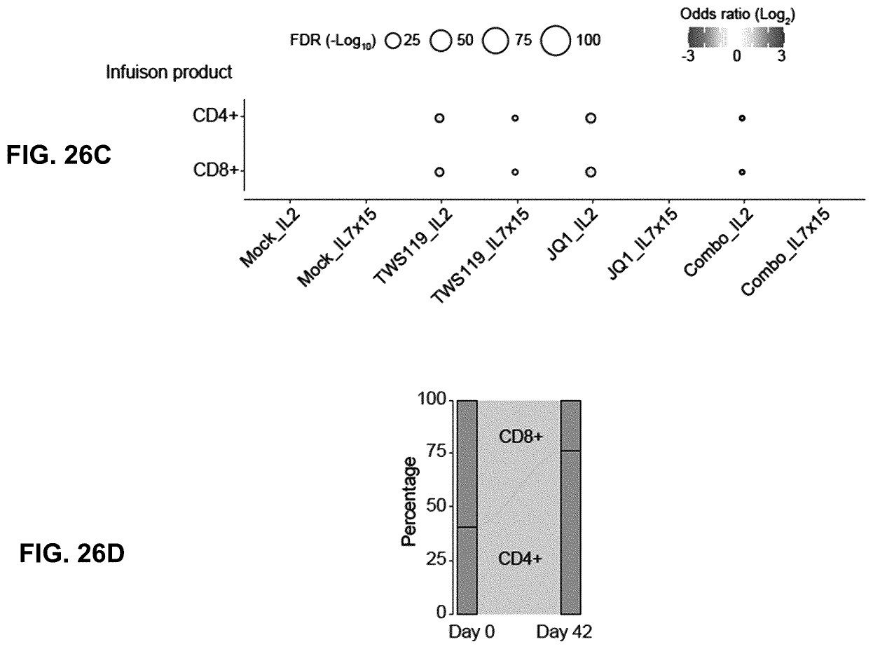

Genetic barcoding using SSN-seq enables longitudinal transcriptional profiling of pooled cells populations. To validate the accuracy and efficiency of SSN-seq in long-term analysis, in vivo evaluation of CD19.28z CAR T cells (generated in previous experiments) was performed (see FIG. 16C). Specifically, the pooled CAR T cells (infusion product) from eight experimental groups were intravenously injected into NSG mice bearing NALM6 acute lymphoblastic leukemia cells expressing bioluminescence reporter to monitor tumor burden (FIG. 17A). To evaluate the pooled CAR T cells persistence and capability to elicit effective recall responses the mice were rechallenged with NALM6 tumor cells three weeks after the CAR T cells administration. Three weeks after the second infusion the CAR T cells (CD45+NGFR+) were isolated from the spleens of treated animals and SSN-seq was performed (FIG. 17A). The in vivo SSN-seq recovered 8,679 high-quality CAR T transcription profiles. Notably, the UMI counts of SSN barcodes detected were comparable among cells from all in vivo groups and within a similar range as in the infusion product (FIG. 17B and FIG. 22A), which demonstrates stability and reliability of SSN barcodes detection ex vivo and in vivo. Correspondingly, over 73% of in vivo CAR T cells were successfully SSN-stratified (FIG. 17C). Further analyses revealed significant changes in distribution of CAR T cells from different treatment groups in the in vivo populations compared to the infusion product (FIG. 17D). Specifically, CAR T cells from the control groups were rarely identified in vivo at the endpoint analysis (day 42) despite comprising over 25% all injected (day 0) cells (FIG. 17D) thus suggesting reduced expansion/persistence (FIG. 26A) which is in agreement with the transcriptional profile predictive of poor long-term therapeutic response (FIGS. 16J and 16K). In contrast to the control, all CAR T treatment groups demonstrated superior in vivo expansion in rechallenged leukemia model. The most significant enrichment of persistent CAR T cells frequency was observed in TWS119+IL2 (28.3-fold), TWS119/JQ1+IL7×15 (10.4-fold), and JQ1+IL2 (9.9-fold) treatment conditions (FIG. 26A). Intriguingly, cell composition analyses revealed that CD8+ and CD4+ CAR T cells within the same treatment groups show distinct expansion patterns (FIG. 17D). Specifically, JQ1+IL2 treatment significantly augmented the CD8+ population, whereas TWS119/JQ1+IL7×15 combination promoted CD4+ cells (FIG. 26B), which was not evident in the infusion product (FIG. 26C). Notably, compared to CAR T cell composition at the time of infusion, a preferred expansion of CD4+ over CD8+ CAR T cells in vivo (FIG. 26D) was noticed, which is congruent with recent clinical findings.

Next, an unsupervised clustering analysis was performed, which revealed 12 distinct populations, with a clear delineation of CD4+ and CD8+ CAR T cells (FIG. 17E). Of note, CD8+ CAR T cell clusters (C11 and C12) were characterized by high T cell dysfunction score (FIG. 17E). Genes for assessing CAR T dysfunction were extracted from a previous study. The analyzed cells were assigned with dysfunction scores using AddModuleScore function by Seurat.

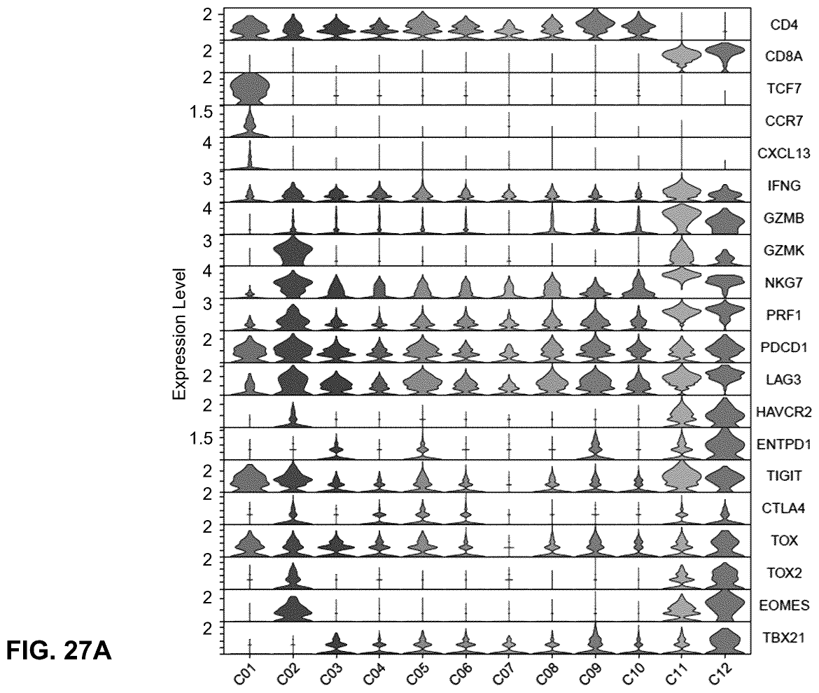

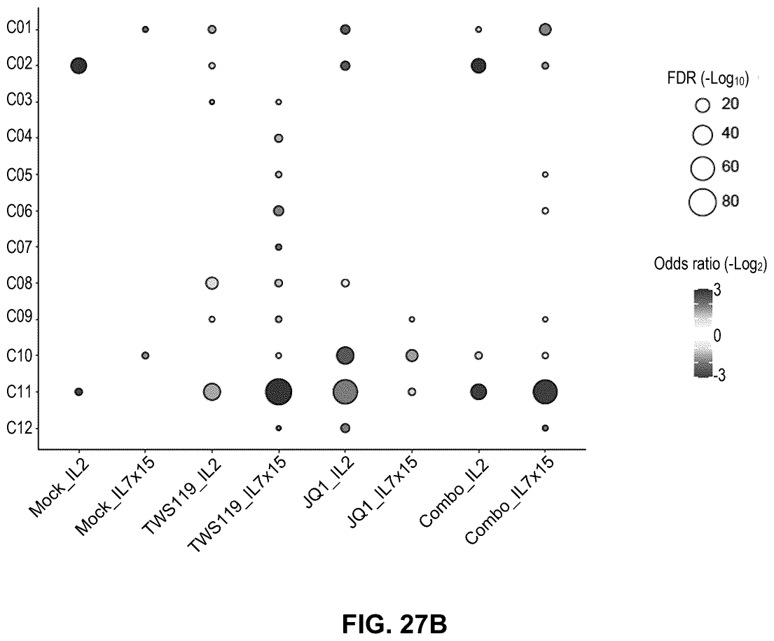

To explore if the identified cell clusters associate with clinically relevant phenotypes, Scissor algorithm was utilized and tumor-infiltrating lymphocytes (TILs) scRNA-seq dataset from melanoma patients treated with PD-1 checkpoint therapy were validated as clinical benchmarks. The Scissor prediction indicated that CD8+ CAR T cell populations (C11 and C12) are associated with lack of response to immune checkpoint blockade (FIG. 17E). Further evaluation of the most upregulated genes in the CD8+ clusters identified several exhaustion makers (LAG3, HAVCR2, EOMES, ID3) and natural killer (NK) cell receptors (KLRC1, KLRD1, KLRK1) (FIG. 17F) in agreement with the recent discovery of NK-like transition in dysfunctional CAR T cells found in patients with diffuse large B-cell lymphoma. In contrast, transcription profile of the CD4+ cluster C01 predicts a favorable clinical response to PD-1 therapy and expression of markers (TCF7, CCR7, CXCL13, GNG4, IGFBP4, IGFL2, PDCD1, and TOX) (FIG. 17F and FIG. 27A) distinctive for tumor-infiltrating TCF7+CD8+ exhausted T cell precursors identified in pan-cancer atlas. TCF7 has been shown to play a central role among naïve, central memory and “progenitor exhausted” CD8+ T cells and is required for effective response upon immune checkpoint blockade. The identification of TCF7+ cluster C01 raised the possibility of the existence of a progenitor population of CD4+ CAR T cells that may respond to immune checkpoint blockade. A newly identified PD-1+TCF1+CD4+ progenitor T cell population was demonstrated to mediate long-term CD4+ T cell responses in the presence of chronic antigen. CAR T cells treated with the combination of TWS119, JQ1 and IL7×15 (Combo_IL7×15) showed the most robust enrichment in the C01 cluster (FIG. 17F and FIG. 27B). Interestingly, TWS119, JQ1, and IL2 (Combo_IL2) treatment also promoted in vivo expansion of CD4+ CAR T cells but was characterized by elevated frequencies of GZMK+C02 cells (FIG. 27A-27B) associated with therapeutic benefit in bladder cancer. These longitudinal observations suggest that modulation of the drug/cytokine conditions during the ex vivo CAR T cell generation alters memory/effector T cell composition with lasting effects for persistent cell expansion in vivo.

Overall, SSN-seq enabled simultaneous comparison of transcriptional profiles of CAR T cells generated using eight different protocols in a single batch of animals and uncovered previously unrecognized impact of chemical perturbation on the CD4+ populations. Although administrated only during the ex vivo CAR T cell manufacture, the small molecule inhibitors treatment resulted in acquisition of distinct transcriptional states with consequences for therapeutic potency of CAR T cells in vivo. Taken together, the coupled ex vivo and in vivo studies underscore the advantage of SSN-seq in longitudinal single-cell transcriptome profiling of pooled samples.

As demonstrated by the studies of human CD19 CAR T cells analyzed at the time of infusion and upon two rounds of tumor challenge in vivo, SSN barcoding robust recovery enables long-term assessment of the effect of different CAR T manufacturing strategies on cell transcriptional states and lineage evolution. The small molecule inhibitors or cytokine treatment during the ex vivo CAR T cell manufacture confirmed previous findings demonstrating that WNT signaling activation or BET inhibition promotes CAR T memory phenotype and leads to enhanced persistence in vivo. In addition, the study revealed that the combination of WNT signaling modulation, BET inhibitors and IL7×15 cytokine treatment of CAR T cells ex vivo supports in vivo expansion of TCF7+CD4+ progenitor lineage, which likely exerts cytolytic effector response in the presence of persistent antigen. The study demonstrated that the culture conditions of ex vivo expansion impact long-term CAR T cells transcription profiles associated with cell lineage, exhaustion and anti-tumor activity in vivo. Coupling pooled SSN barcoding with transcriptome profiling provides a powerful approach to assess the effect of non-genetic perturbations (e.g. inhibitors) on cell state but can be leveraged as genetic labels for pooled cDNA screens to facilitate single-cell gain-of-function studies. The pooled SSN-seq barcoding will also enable head-to-head competition screens for accelerated validation of optimized CAR designs or additional modulators of critical T cell functions for effective therapies.

### Example 11. Personalized Medicine with B Cell Receptor and T Cell Receptor Administration (Prophetic)

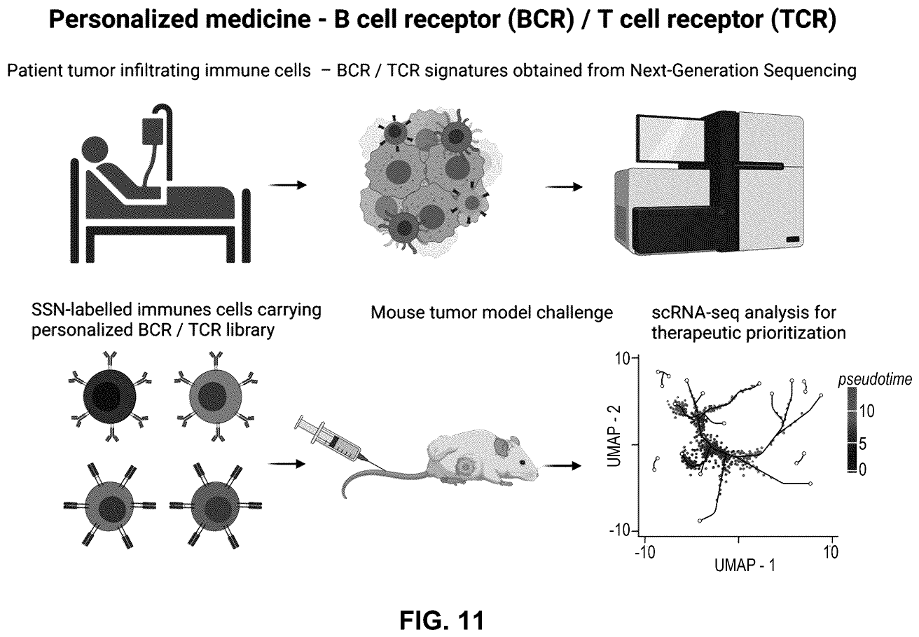

Tumor infiltrating immune cells from a patient are analyzed. First, tumor infiltrating immune cells with unique B cell receptor and T cell receptor signatures are isolated from a patient. Next, the tumor infiltrating immune cells are labelled by Shielded Small Nucleotides and injected into a mouse tumor model. After a sufficient time period, the tumor is extracted from the mouse. Single-cell RNA sequencing is then performed to analyze the tumor infiltrating immune cells for therapeutic prioritization. See FIG. 11. The shielded sgRNA barcode labelling can allow the sample to be demultiplexed.

### Example 12. Transplant Based Tumor Model (Prophetic)

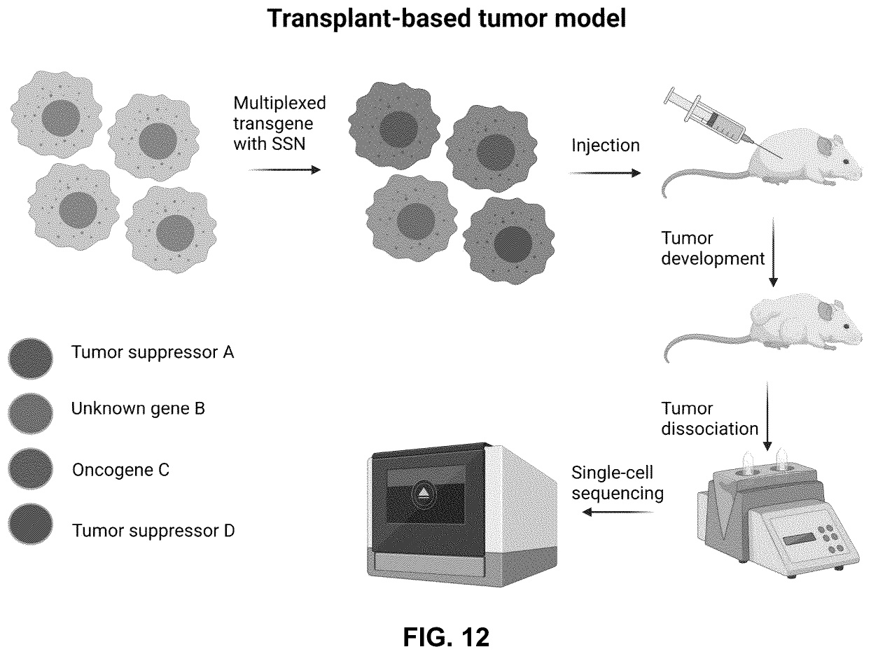

SSNs can be tested for transplant based tumor models. Cancer cells with different genetic variations are isolated and labelled by Shielded Small Nucleotides. Next, the labelled cancer cells are injected into a mouse model, upon which a tumor forms after a sufficient time period. The tumor is isolated, upon which the tumor cells are analyzed by sc-RNA sequencing. See FIG. 12. The phenotypes of the cancer cells are analyzed and new tumor suppressors and oncogenes can be profiled. The shielded sgRNA barcode labelling can allow the sample to be demultiplexed.

### Example 13. Autochthonous Tumor Model (Prophetic)

SSNs can be used to generate autochthonous tumor models for research as well. Viral vectors including different oncogenes are labelled by Shielded Small Nucleotides. The vectors are injected into a mouse model, upon which a tumor forms after a sufficient time period. Next, the tumor is isolated, upon which the tumor cells are analyzed by sc-RNA sequencing. See FIG. 13. The phenotypes of the cancer cells are analyzed and new tumor suppressors and oncogenes can be profiled. The shielded sgRNA barcode labelling can allow the sample to be demultiplexed.

1. A barcoded RNA comprising:
(a) a first shield sequence at the 5′ end of the barcoded RNA;
(b) a barcode sequence;
(c) a scaffold sequence;
(d) a capture sequence; and
(e) a second shield sequence at the 3′ end of the barcoded RNA.

2. The barcoded RNA of claim 1, wherein the first shield sequence and/or second shield sequence comprises at least one stem loop.

3. The barcoded RNA of claim 1, wherein:
(a) the first shield sequence comprises a sequence at least 85%, at least 90%, at least 95%, or 100% identical to the nucleotide sequence set forth as SEQ ID NO: 10;
(b) the barcode sequence is 8 to 20 nucleotides long;
(c) the scaffold sequence comprises a sgRNA or a bacteriophage pRNA;
(d) the capture sequence comprises a sequence at least 85%, at least 90%, at least 95%, or 100% identical to the nucleotide sequence set forth as SEQ ID NO: 12; and/or
(e) the second shield sequence comprises a sequence at least 85%, at least 90%, at least 95%, or 100% identical to the nucleotide sequence set forth as SEQ ID NO: 11.

4.-10. (canceled)

11. The barcoded RNA of claim 3, wherein the bacteriophage pRNA is F29 or F30.

12.-14. (canceled)

15. The barcoded RNA of claim 1, further comprising a terminator sequence and/or a RNA aptamer.

16. (canceled)

17. The barcoded RNA of claim 15, wherein the RNA aptamer is a fluorescent RNA aptamer, wherein the fluorescent RNA aptamer is a Broccoli RNA aptamer.

18. (canceled)

19. A method of performing single-cell RNA sequencing comprising
(a) introducing a barcoded RNA library to a population of cells, wherein the barcoded RNA library comprises a plurality of barcoded RNA constructs comprising (i) a first shield sequence at the 5′ end of the barcoded RNA, (ii) a unique barcode sequence, (iii) a scaffold sequence, (iv) a capture sequence, and (v) a second shield sequence at the 3′ end of the barcoded RNA; and
(b) performing single-cell RNA sequencing on the population of cells, wherein the cell can be identified by the unique barcode sequence, and wherein an individual cell has a gene expression profile.

20.-47. (canceled)

48. A polynucleotide comprising a promoter operably linked to a nucleic acid encoding the barcoded RNA of claim 1.

49.-50. (canceled)

51. The polynucleotide of claim 48, wherein the nucleic acid is positioned between two inverted terminal repeats (ITRs).

52. The polynucleotide of claim 48, wherein the promoter is a constitutively active promoter, a cell-type specific promoter, or an inducible promoter.

53. The polynucleotide of claim 48, wherein the promoter is a Pol III promoter, wherein the Pol III promoter is a U6 promoter.

54.-71. (canceled)

72. A method of multiplexing samples for single cell sequencing comprising:
(a) labeling single cells from a plurality of samples with the barcoded RNA of claim 1, wherein the barcode sequence comprises a unique barcode sequence and a cell of origin barcode sequence;
(b) constructing a multiplexed single cell sequencing library for the plurality of samples comprising the cell of origin barcodes.

73. (canceled)

74. A method of detecting a gene expression profile of CAR-T cells comprising:
(a) transducing T cells with a Chimeric Antigen Receptor (CAR) and at least one barcoded RNA construct to form a population of CAR-T cells, wherein the barcoded RNA construct comprises (i) a 5′ shield sequence, (ii) a unique barcode sequence, (iii) a scaffold sequence, (iv) a capture sequence, and (v) a 3′ shield sequence;
(b) subjecting the population of CAR-T cells to a test condition;
(c) collecting the population of CAR-T cells after the test condition;
(d) pooling the population of CAR-T cells; and
(e) performing single-cell RNA sequencing to determine a gene expression profile for an individual CAR-T cell, wherein the unique barcode sequence allows for demultiplexing of the population of CAR-T cells.

75.-81. (canceled)

82. A method of selecting a tumor infiltrating immune cell from a patient comprising:
(a) isolating tumor infiltrating immune cells from a patient;
(b) introducing the barcoded RNA of claim 1 to the tumor infiltrating immune cells, wherein the barcoded sequence is a unique barcode sequence;
(c) challenging the tumor infiltrating immune cells with cancer cells;
(d) collecting the tumor infiltrating immune cells after the challenge;
(e) pooling the population of tumor infiltrating immune cells and performing single-cell RNA sequencing to determine a gene expression profile for an individual tumor infiltrating immune cell, wherein the unique barcode sequence allows for demultiplexing of the population of CAR-T cells; and
(f) selecting a tumor infiltrating immune cell with the gene expression profile desired for treatment of the patient.

83.-87. (canceled)

88. A method for analyzing tumor development comprising:
(a) introducing at least one barcoded RNA of claim 1 to a population of cancer cells to form a sample population, wherein the barcode sequence is a unique barcode sequence;
(b) injecting the sample population into an animal model;
(c) allowing a tumor to develop in the animal model;
(d) isolating the tumor from the animal model;
(e) performing single-cell RNA sequencing on cells in the tumor, wherein the unique barcode sequence allows for demultiplexing of the sample.

89.-90. (canceled)

91. A method for analyzing oncogenes comprising:
(a) introducing a viral vector to an animal model, wherein the viral vector comprises a unique oncogene and the barcoded RNA of claim 1, sequence, (ii) wherein the barcode sequence is a unique barcode sequence;
(b) allowing a tumor to develop in the animal model;
(c) isolating the tumor from the animal model;
(d) performing single-cell RNA sequencing on cells in the tumor, wherein the unique barcode sequence allows for demultiplexing of the sample.

92. (canceled)

93. A cell expressing a barcoded RNA of claim 1.

94. A library comprising a plurality of barcoded RNAs comprising one or more of the barcoded RNAs of claim 1.

95.-100. (canceled)

101. A kit comprising an expression construct comprising a promoter operably linked to a nucleic acid encoding one or more of the barcoded RNA of claim 1.

102.-108. (canceled)

109. A method of transcriptional profiling, the method comprising a) introducing a barcoded RNA library to a population of cells, wherein the barcoded RNA library comprises at least one of the barcoded RNAs of claim 1, wherein the barcode sequence is a unique barcode sequence; b) performing single-cell RNA sequencing on the population of cells, wherein the cell can be identified by the unique barcode sequence, and wherein an individual cell has a gene expression profile; and c) lineage-tracing and transcriptional profiling the individual cell of the population of cells.

110.-112. (canceled)
